(19) United States
(12) Patent Application Publication
(10) Pub. No.: US 2020/0157138 A1
(43) Pub. Date: May 21, 2020

(54) NICOTINAMIDE ADENINE DINUCLEOTIDE ANALOGUES

(71) Applicant: University of Southern California, Los Angeles, CA (US)

(72) Inventors: Yong Zhang, Los Angeles, CA (US); Xiao-Nan Zhang, Los Angeles, CA (US)

(21) Appl. No.: 16/615,223

(22) PCT Filed: Jun. 8, 2018
(86) PCT No.: PCT/US2018/036778

§ 371 Date: Nov. 20, 2019

Related U.S. Application Data
(60) Provisional application No. 62/517,784, filed on Jun. 9, 2017.
(60) Provisional application No. 62/529,989, filed on Jul. 7, 2017.

(51) Int. Cl. C07H 19/207; G01N 33/573

(52) U.S. Cl. CPC C07H 19/207; G01N 33/573; G01N 2333/91142; G01N 2440/40; G01N 2458/00

(57) ABSTRACT

Provided herein are nicotinamide adenine dinucleotide analogues, compositions comprising such compounds, and methods of using such analogues and compositions.

### CROSS-REFERENCE TO RELATED APPLICATIONS

This application claims priority under 35 U.S.C. § 119(e) to U.S. Provisional Patent Application No. 62/517,784, filed Jun. 9, 2017, and U.S. Provisional Patent Application No. 62/529,989, filed Jul. 7, 2017, the disclosures each of which are incorporated by reference in their entireties.

### BACKGROUND

Numerous biological processes are orchestrated by protein post-translational modifications (PTMs). Among key PTMs is protein ADP-ribosylation catalyzed by a superfamily of enzymes named ADP-ribosyltransferases (ARTs) with nicotinamide adenine dinucleotide (NAD⁺) as a cofactor. The human genome is found to encode 20 ART enzymes including intracellular poly-ADP-ribose polymerases (PARPs), sirtuins (SIRTs), and extracellular ART1-5, which possess poly- or mono-ADP-ribosylation activity.

Protein ADP-ribosylation is shown to play vital roles in regulating genome stability, protein homeostasis, cell proliferation, differentiation, and apoptosis. Abnormally increased ARTs activities are causatively linked with various human diseases such as cancer, immune disorders, and neurodegenerative diseases. However, the cellular functions and physiological and pathophysiological roles for most PARPs have remained elusive.

Conventional approaches require invasive procedures for imaging ADP-ribosylation in live cells and are incapable of dissecting ADP-ribosylated networks at physiological and pathophysiological conditions. Accordingly, non-invasive procedures for imaging ADP-ribosylation in live cells are needed.

### SUMMARY

In some aspects, provided is a compound of Formula (I-A):

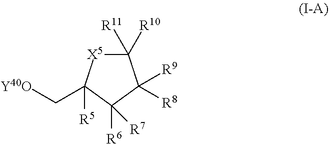

or a tautomer thereof, or an N-oxide of each thereof, or a pharmaceutically acceptable salt of each of the aforementioned, or a pharmaceutically acceptable solvate of each of the foregoing, wherein: each of R⁵, R⁶, R⁷, R⁸, R⁹, R¹⁰, and R¹¹ independently is a hydrogen, —N₃, a hydroxyl, an optionally substituted C₁-C₁₀ alkyl, an optionally substituted C₂-C₁₀ alkynyl, an optionally substituted C₁-C₁₀ alkoxy, —SR³⁰, an optionally substituted C₆-C₁₀ aryl, an optionally substituted 5-15 membered heteroaryl, or Z; X⁵ is —S—, —O—, or —NR²⁰—; each R²⁰ and R³⁰ is independently a hydrogen or an optionally substituted C₁-C₁₀ alkyl; Z is

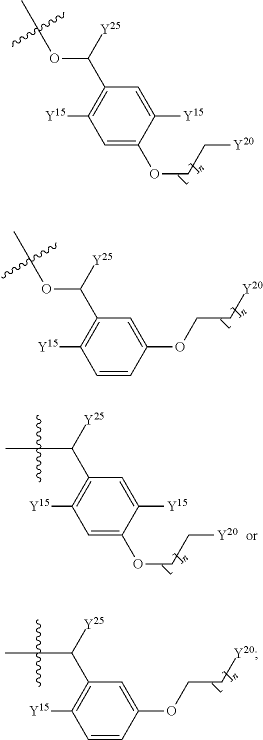

each n is independently 1-4 or 1, 3, or 4; each Y¹⁵ is independently a hydrogen, —NO₂, a halo, a cyano, a hydroxyl, an optionally substituted C₁-C₆ alkyl, or an optionally substituted C₁-C₆ alkoxy; each Y²⁵ is independently a hydrogen or an optionally substituted C₁-C₆ alkyl, and each Y²⁰ is independently selected from the group consisting of:

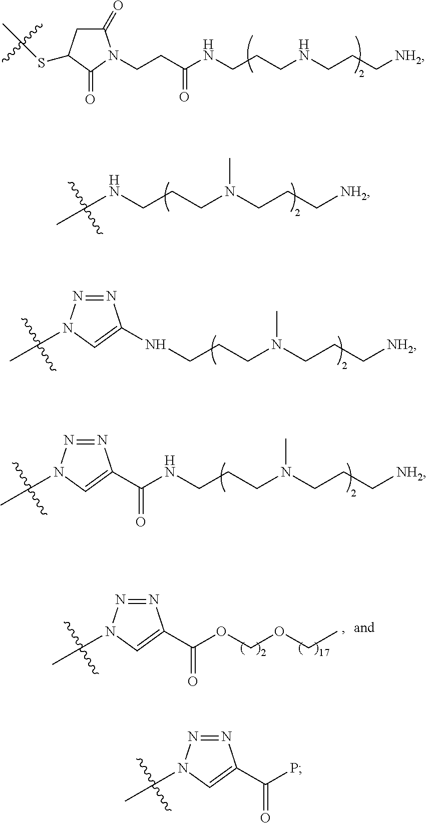

P is a cationic polypeptide of about 5 to 30 amino acid residues in length; Y⁴⁰ is a hydrogen, an optionally substituted C₁-C₆ alkyl, an optionally substituted C₂-C₁₀ alkenyl, an optionally substituted C₂-C₁₀ alkynyl, an optionally substituted C₆-C₁₀ aryl, an optionally substituted 5-15 membered heteroaryl, or -L¹Y³⁵; L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—; each R¹⁰⁰ is independently —O^{⊖}, an optionally substituted C₁-C₁₀ alkyl group, or an optionally substituted C₁-C₁₀ alkoxy; and Y³⁵ is a hydroxyl or an optionally substituted C₁-C₆ alkoxy.

In some aspects, provided is a compound of Formula (I-B):

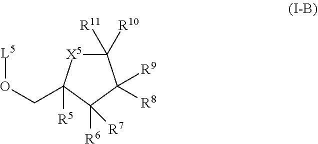

or a tautomer thereof, or an N-oxide of each thereof, or a pharmaceutically acceptable salt of each of the aforementioned, or a pharmaceutically acceptable solvate of each of the foregoing, wherein: each of R⁵, R⁶, R⁷, R⁸, R⁹, R¹⁰, and R¹¹ independently is a hydrogen, —N₃, a hydroxyl, an optionally substituted C₁-C₁₀ alkyl, an optionally substituted C₂-C₁₀ alkynyl, an optionally substituted C₁-C₁₀ alkoxy, —SR³⁰, an optionally substituted C₆-C₁₀ aryl, an optionally substituted 5-15 membered heteroaryl, or Z; X⁵ is —S—, —O—, or —NR²⁰—; each R²⁰ and R³⁰ is independently a hydrogen or an optionally substituted C₁-C₁₀ alkyl; Z is

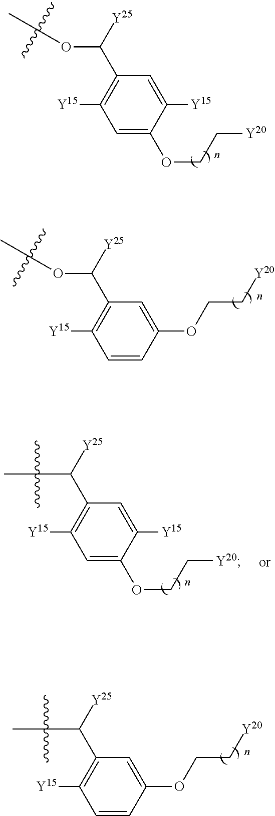

each n is independently 1-4 or 1, 3, or 4; each Y¹⁵ is independently a hydrogen, —NO₂, a halo, a cyano, a hydroxyl, an optionally substituted C₁-C₆ alkyl, or an optionally substituted C₁-C₆ alkoxy; each Y²⁵ is independently a hydrogen or an optionally substituted C₁-C₆ alkyl, and

each Y²⁰ is independently selected from the group consisting of:

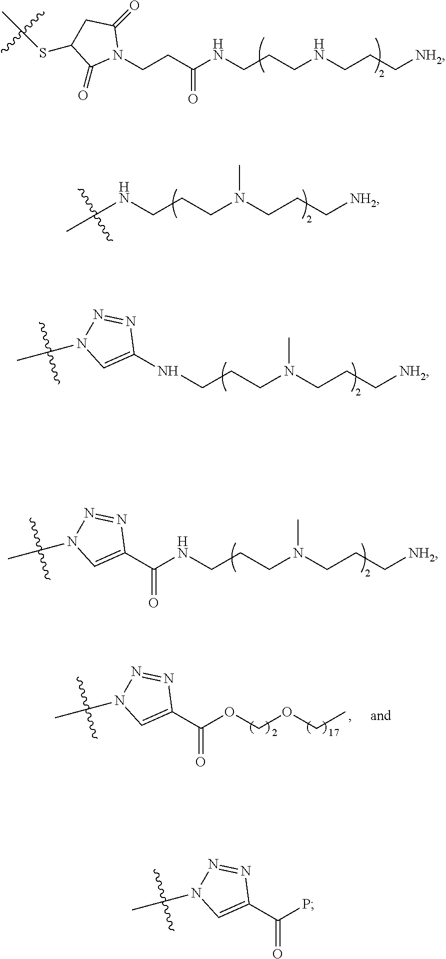

P is a cationic polypeptide of about 5-30 amino acid residues in length; L⁵ is a hydrogen, an optionally substituted C₁-C₆ alkyl, or -L¹Y³⁵; L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—; each R¹⁰⁰ is independently —O^{⊖}, an optionally substituted C₁-C₁₀ alkyl group, or an optionally substituted C₁-C₁₀ alkoxy; and Y³⁵ is a hydroxyl or an optionally substituted C₁-C₆ alkoxy.

In some aspects, provided is a compound of Formula (I-C):

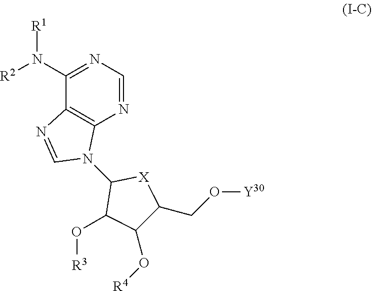

or a tautomer thereof, or an N-oxide of each thereof, or a pharmaceutically acceptable salt of each of the aforementioned, or a pharmaceutically acceptable solvate of each of the foregoing, wherein each R¹, R², R³, and R⁴ independently is a hydrogen or an optionally substituted C₁-C₆ alkyl or Z; X is —S—, —O—, or —NR²⁰—; R²⁰ is a hydrogen or an optionally substituted C₁-C₁₀ alkyl; Z is

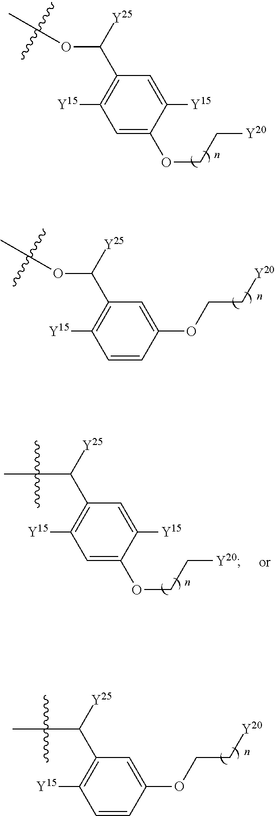

each n is independently 1-4 or 1, 3, or 4; each Y¹⁵ is independently a hydrogen, —NO₂, a halo, a cyano, a hydroxyl, an optionally substituted C₁-C₆ alkyl, or an optionally substituted C₁-C₆ alkoxy; each Y²⁵ is independently a hydrogen or an optionally substituted C₁-C₆ alkyl, and each Y²⁰ is independently selected from the group consisting of:

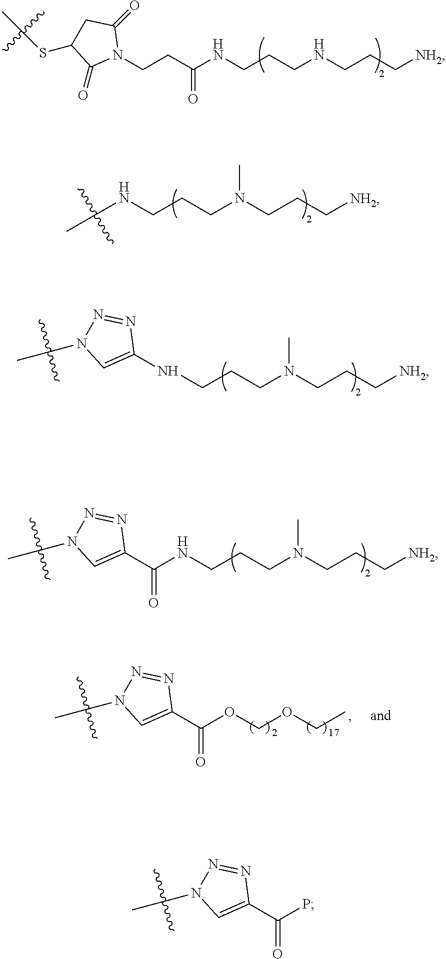

P is a cationic polypeptide of about 5-30 amino acid residues; Y³⁰ is a hydrogen, an optionally substituted C₁-C₆ alkyl, an optionally substituted C₂-C₁₀ alkenyl, an optionally substituted C₂-C₁₀ alkynyl, an optionally substituted C₆-C₁₀ aryl, an optionally substituted 5-15 membered heteroaryl, or -L¹Y³⁵; L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—; each R¹⁰⁰ is independently —O^{⊖}, an optionally substituted C₁-C₁₀ alkyl group, or an optionally substituted C₁-C₁₀ alkoxy; and Y³⁵ is a hydroxyl or an optionally substituted C₁-C₆ alkoxy.

In some aspects provided is a compound of Formula (I-D):

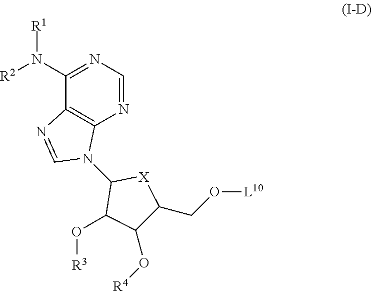

or a tautomer thereof, or an N-oxide of each thereof, or a pharmaceutically acceptable salt of each of the aforementioned, or a pharmaceutically acceptable solvate of each of the foregoing, wherein: each of R¹, R², R³, and R⁴ independently is a hydrogen or an optionally substituted C₁-C₆ alkyl or Z; X is —S—, —O—, or —NR²⁰—; R²⁰ is a hydrogen or an optionally substituted C₁-C₁₀ alkyl; L¹⁰ is a hydrogen, an optionally substituted C₁-C₆ alkyl, or -L¹Y³⁵;

L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—; each R¹⁰⁰ is independently —O^{⊖}, an optionally substituted C₁-C₁₀ alkyl group, or an optionally substituted C₁-C₁₀ alkoxy; Y³⁵ is a hydroxyl or an optionally substituted C₁-C₆ alkoxy; Z is

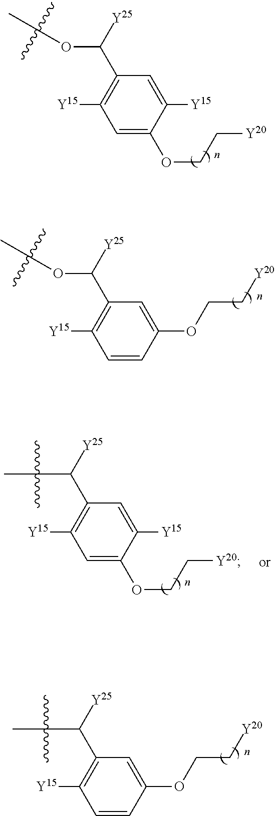

each n is independently 1-4 or 1, 3, or 4; each Y¹⁵ is independently a hydrogen, —NO₂, a halo, a cyano, a hydroxyl, an optionally substituted C₁-C₆ alkyl, or an optionally substituted C₁-C₆ alkoxy; each Y²⁵ is independently a hydrogen or an optionally substituted C₁-C₆ alkyl, and each Y²⁰ is independently selected from the group consisting of:

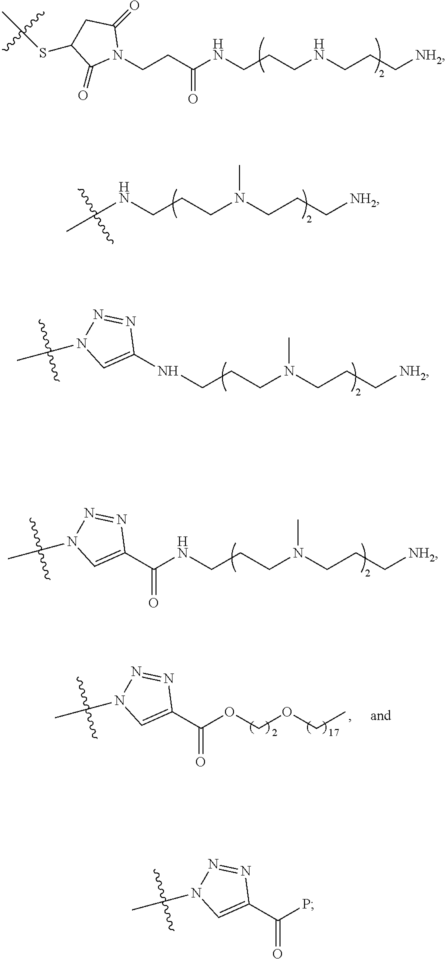

P is a cationic polypeptide of about 5-30 amino acid residues in length.

In some aspects, provided is a compound of Formula (I):

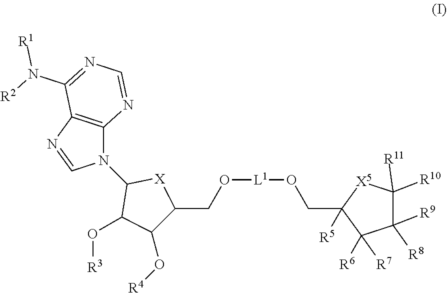

or a tautomer thereof, or an N-oxide of each thereof, or a pharmaceutically acceptable salt of each of the aforementioned, or a pharmaceutically acceptable solvate of each of the foregoing, wherein: each R¹, R², R³, and R⁴ independently is a hydrogen or an optionally substituted C₁-C₆ alkyl or Z; X is —S—, —O—, or —NR²⁰—; X⁵ is —S—, —O—, or —NR²⁰—; L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—; R¹⁰⁰ is —O^{⊖}, an optionally substituted C₁-C₁₀ alkyl group, or an optionally substituted C₁-C₁₀ alkoxy; each R⁵, R⁶, R⁷, R⁸, R⁹, R¹⁰, and R¹¹ independently is a hydrogen, —N₃, a hydroxyl, an optionally substituted C₁-C₁₀ alkyl, an optionally substituted C₂-C₁₀ alkynyl, an optionally substituted C₁-C₁₀ alkoxy, —SR³⁰, an optionally substituted C₆-C₁₀ aryl, an optionally substituted 5-15 membered heteroaryl, or Z; Z is

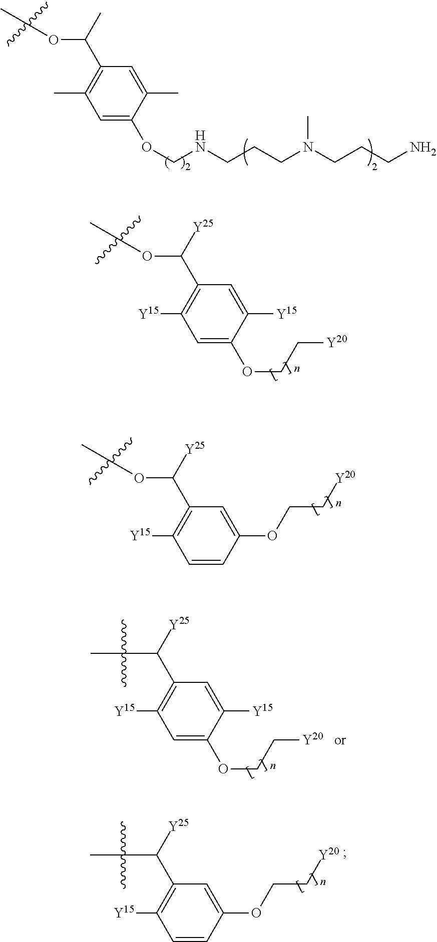

each n is independently 1-4 or 1, 3, or 4; each Y¹⁵ is independently a hydrogen, —NO₂, a halo, a cyano, a hydroxyl, an optionally substituted C₁-C₆ alkyl, or an optionally substituted C₁-C₆ alkoxy; each Y²⁵ is independently a hydrogen or an optionally substituted C₁-C₆ alkyl, and each Y²⁰ is independently selected from the group consisting of:

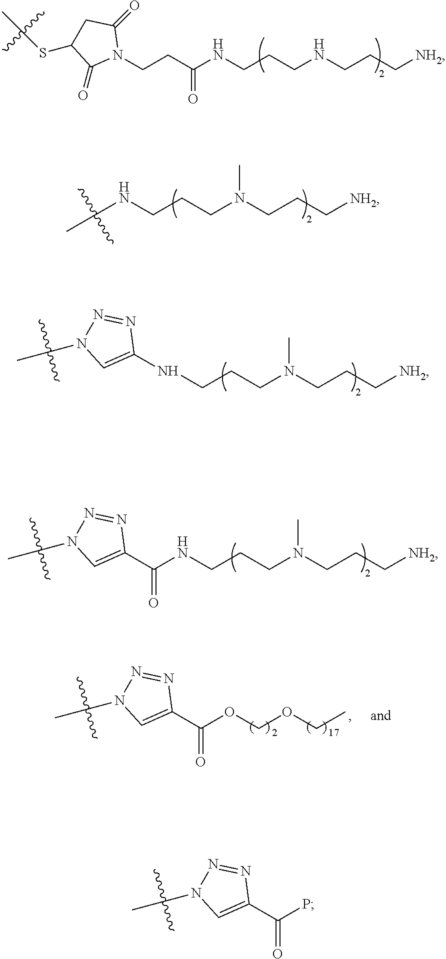

P is a cationic polypeptide of 9-30 amino acid residues in length; and each R²⁰ and R³⁰ is independently a hydrogen or an optionally substituted C₁-C₁₀ alkyl.

This disclosure also provides a compound of Table 1, 2, 3 or 4.

This disclosure also provides a method of monitoring and/or tracking ADP-ribosylation in a cell or sample comprising a PARP enzyme, the method comprising contacting the cell or sample with a compound of as disclosed above under conditions that favor a PARP catalyzed reaction to produce a reaction product; labeling a PARP catalyzed reaction product; and detecting the product of the PARP catalyzed reaction, thereby monitoring and/or tracking ADP-ribosylation. In one aspect, click chemistry is used to label the reaction product.

Also provided is a method of purifying a PARP substrate protein, the method comprising: contacting a cell or sample comprising PARP with a compound as disclosed herein under conditions that favor a PARP catalyzed reaction; labeling a PARP catalyzed reaction product with an affinity label, and purifying the product of the PARP catalyzed reaction by selecting for the affinity labeled product. In one aspect, click chemistry is used to label the reaction product.

In some aspects, provided is a method of identifying a protein as a PARP substrate, the method comprising contacting a cell or sample comprising the PARP with a compound as disclosed herein under conditions that favor a PARP catalyzed reaction; labeling a PARP catalyzed reaction product with an affinity label; and purifying and characterizing the product of the PARP catalyzed reaction being bound to the affinity label. In one aspect, click chemistry is used to label the reaction product.

In some aspects, provided is a method of labeling a PARP substrate protein, the method comprising contacting a cell or sample comprising PARP with a compound as disclosed herein under conditions that favor a PARP catalyzed reaction; and labeling a product of a PARP catalyzed reaction. In one aspect, click chemistry is used to label the product.

Methods to prepare the compounds as disclosed herein are further provided.

Also provided herein are kits comprising one or more compounds as disclosed herein and instructions for use. Optionally reagents for carrying out the methods as disclosed herein are further provided in the kits.

### BRIEF DESCRIPTION OF THE DRAWINGS

FIG. 1 is a schematic showing novel molecular tools for studying ADP-ribosylation in live cells. Cell-permeable nicotinamide (Nam) riboside (NR) analogues enable in situ generation of clickable nicotinamide adenine dinucleotide (NAD⁺) analogues through NR kinase (NRK) and nicotinamide mononucleotide adenylyltransferase (NMNAT). NAD⁺ analogues generally recognized by native PARPs allow non-invasive tracking of cellular ADP-ribosylation.

FIG. 2 shows a cellular imaging of ADP-ribosylation using generated NR1 analogue (compound 8 in Scheme 1). HeLa cells were cultured in growth medium supplemented with 0.1 or 1 mM NR1 for 48 hr, followed by labeling with fluorescent dye via click chemistry.

FIGS. 3A-3B are visualizations of cellular ADP-ribosylation through the generated NR1 analogue. HeLa cells were cultured in growth medium supplemented with NR1 at indicated concentrations for 6-12 hr in the absence or presence of topotecan and 6-(5H)-phenanthridinone, followed by labeling with fluorescent dye via click chemistry.

FIG. 4 shows an immunoblot analysis of lysates of Expi293 cells treated with 1 mM NR, and 1 mM NR1 analogue for 12 hr in the absence or presence of varied concentrations of topotecan.

FIGS. 5A-5B illustrate the in vitro biosynthesis of NAD1 analogue by NRK1 and NMNAT1. FIG. 5A shows the SDS-PAGE gel of purified NRK1 and NMNAT1 from E. coli. FIG. 5B shows the HPLC chromatographic analysis of the time-dependent generation of NAD1 analogue catalyzed by purified NRK1 and NMNAT1.

FIGS. 6A-6B show the LC-MS analysis of NR1 in the cellular extracts of Expi293 cells treated with 10 mM NR1 for 10 hr. FIG. 6A shows the reverse-phase liquid chromatography for separation of the cellular extracts. FIG. 6B shows the mass spectrometry of the selected fraction for detection of cellular NR1 analogue.

FIGS. 7A-7B show the LC-MS analysis of NAD1 in the cellular extracts of Expi293 cells treated with 10 mM NR1 for 10 hr. FIG. 7A shows the reverse-phase liquid chromatography for separation of the cellular extracts. FIG. 7B shows the mass spectrometry of the selected fraction for detection of cellular NAD+1.

FIG. 8 shows the MS (ESI) of the reaction to synthesize NAD+27; the units for the X-axis are: m/z, Da, and the units for the Y-axis are: intensity, cps.

### DETAILED DESCRIPTION

Throughout this disclosure, various publications, patents and published patent specifications are referenced by an identifying citation. The disclosures of these publications, patents and published patent specifications are hereby incorporated by reference into the present disclosure in their entirety to more fully describe the state of the art to which this invention pertains.

### Definitions

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd}edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

“Topotecan” is a compound that induces cellular DNA damage that would activate PARP activity in the cells. In the presence of topotecan, the cells treated by the compounds of the disclosure show increased fluorescence activity in the nucleus. This demonstrates the utility of the compounds in visualizing the cellular ADP-ribosylation catalyzed by PARP enzymes.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkenyl” refers to monovalent straight or branched hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxyl, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein and with the proviso that any hydroxyl or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein and with the proviso that any hydroxyl or thiol substitution is not attached to an acetylenic carbon atom.

“Alkylene” refers to divalent saturated aliphatic hydrocarbyl groups preferably having from 1 to 6 and more preferably 1 to 3 carbon atoms that are either straight-chained or branched. This term is exemplified by groups such as methylene (—CH₂—), ethylene (—CH₂CH₂—), n-propylene (—CH₂CH₂CH₂—), iso-propylene (—CH₂CH(CH₃)— or —CH(CH₃)CH₂—), butylene (—CH₂CH₂CH₂CH₂—), isobutylene (—CH₂CH(CH₃)CH₂—), sec-butylene (—CH₂CH₂(CH₃)CH—), and the like. Similarly, “alkenylene” and “alkynylene” refer to an alkylene moiety containing respective 1 or 2 carbon carbon double bonds or a carbon carbon triple bond.

“Substituted alkylene” refers to an alkylene group having from 1 to 3 hydrogens replaced with substituents selected from the group consisting of alkyl, substituted alkyl, alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl ester, cycloalkyl, substituted cycloalkyl, heteroaryl, substituted heteroaryl, heterocyclic, substituted heterocyclic, and oxo wherein said substituents are defined herein.

In some embodiments, the alkylene has 1 to 2 of the aforementioned groups, or having from 1-3 carbon atoms replaced with —O—, —S—, or —NR^{Q}— moieties where R^{Q} is H or C₁-C₆ alkyl. It is to be noted that when the alkylene is substituted by an oxo group, 2 hydrogens attached to the same carbon of the alkylene group are replaced by “═O”. “Substituted alkenylene“and” substituted alkynylene” refer to alkenylene and substituted alkynylene moieties substituted with substituents as described for substituted alkylene.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR⁴⁷C(O)alkyl, —NR⁴⁷C(O)substituted alkyl, —NR⁴⁷C(O)cycloalkyl, —NR⁴⁷C(O)substituted cycloalkyl, —NR⁴⁷C(O)cycloalkenyl, —NR⁴⁷C(O)substituted cycloalkenyl, —NR⁴⁷C(O)alkenyl, —NR⁴⁷C(O)substituted alkenyl, —NR⁴⁷C(O)alkynyl, —NR⁴⁷C(O)substituted alkynyl, —NR⁴⁷C(O)aryl, —NR⁴⁷C(O)substituted aryl, —NR⁴⁷C(O)heteroaryl, —NR⁴⁷C(O)substituted heteroaryl, —NR⁴⁷C(O)heterocyclic, and —NR⁴⁷C(O)substituted heterocyclic wherein R⁴⁷ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

An animal, subject or patient for diagnosis or treatment refers to an animal such as a mammal, or a human, ovine, bovine, feline, canine, equine, simian, etc. Non-human animals subject to diagnosis or treatment include, for example, simians, murine, such as, rat, mice, canine, leporid, livestock, sport animals, and pets.

“Amino” refers to the group —NH₂.

“Substituted amino” refers to the group —NR⁴⁸R⁴⁹ where R⁴⁸ and R⁴⁹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R⁴⁸ and R⁴⁹ are optionally joined, together with the nitrogen bound thereto to form a heterocyclic or substituted heterocyclic group, provided that R⁴⁸ and R⁴⁹ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R⁴⁸ is hydrogen and R⁴⁹ is alkyl, the substituted amino group is sometimes referred to herein as alkylamino. When R⁴⁸ and R⁴⁹ are alkyl, the substituted amino group is sometimes referred to herein as dialkylamino. When referring to a monosubstituted amino, it is meant that either R⁴⁸ or R⁴⁹ is hydrogen but not both. When referring to a disubstituted amino, it is meant that neither R⁴⁸ nor R⁴⁹ are hydrogen.

“Aminocarbonyl” refers to the group —C(O)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR⁴⁷C(O)NR⁵⁰R⁵¹ where R⁴⁷ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic, and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NR⁴⁷C(S)NR⁵⁰R⁵¹ where R⁴⁷ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR⁴⁷SO₂NR⁵⁰R⁵¹ where R⁴⁷ is hydrogen or alkyl and R⁵ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR⁵²)NR⁵⁰R⁵¹ where R⁵⁰, R⁵¹, and R⁵² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-Y¹, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Azide” refers to the group —N═N^{⊕}═N^{⊖}.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR⁴⁷C(O)O-alkyl, —NR⁴⁷C(O)O-substituted alkyl, —NR⁴⁷C(O)O-alkenyl, —NR⁴⁷C(O)O-substituted alkenyl, —NR⁴⁷C(O)O-alkynyl, —NR⁴⁷C(O)O-substituted alkynyl, —NR⁴⁷C(O)O-aryl, —NR⁴⁷C(O)O-substituted aryl, —NR⁴⁷C(O)O-cycloalkyl, —NR⁴⁷C(O)O-substituted cycloalkyl, —NR⁴⁷C(O)O-cycloalkenyl, —NR⁴⁷C(O)O-substituted cycloalkenyl, —NR⁴⁷C(O)O-heteroaryl, —NR⁴⁷C(O)O-substituted heteroaryl, —NR⁴⁷C(O)O-heterocyclic, and —NR⁴⁷C(O)O-substituted heterocyclic wherein R⁴⁷ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

A “composition” as used herein, intends an active agent, such as a compound as disclosed herein and a carrier, inert or active. The carrier can be, without limitation, solid such as a bead or resin, or liquid, such as phosphate buffered saline.

Administration or treatment in “combination” refers to administering two agents such that their pharmacological effects are manifest at the same time. Combination does not require administration at the same time or substantially the same time, although combination can include such administrations.

“Cyano” refers to the group —CN.

“Cycloalkyl” refers to cyclic alkyl groups of from 3 to 10 carbon atoms having single or multiple cyclic rings including fused, bridged, and spiro ring systems. The fused ring can be an aryl ring provided that the non aryl part is joined to the rest of the molecule. Examples of suitable cycloalkyl groups include, for instance, adamantyl, cyclopropyl, cyclobutyl, cyclopentyl, and cyclooctyl.

“Cycloalkenyl” refers to non-aromatic cyclic alkyl groups of from 3 to 10 carbon atoms having single or multiple cyclic rings and having at least one >C═C<ring unsaturation and preferably from 1 to 2 sites of >C═C<ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thioxo, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Cycloalkyloxy” refers to —O-cycloalkyl.

“Substituted cycloalkyloxy refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR⁵³C(═NR⁵³)N(R⁵³)₂ where each R⁵³ is independently selected from the group consisting of hydrogen, alkyl, substituted alkyl, aryl, substituted aryl, heteroaryl, substituted heteroaryl, cycloalkyl, substituted cycloalkyl, heterocyclic, and substituted heterocyclic and two R⁵³ groups attached to a common guanidino nitrogen atom are optionally joined together with the nitrogen bound thereto to form a heterocyclic or substituted heterocyclic group, provided that at least one R⁵³ is not hydrogen, and wherein said substituents are as defined herein.

“Halo” or “halogen” refers to fluoro, chloro, bromo and iodo.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Certain non-limiting examples include pyridinyl, pyrrolyl, indolyl, thiophenyl, oxazolyl, thizolyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through a non-aromatic ring. In one embodiment, the nitrogen and/or sulfur atom(s) of the heterocyclic group are optionally oxidized to provide for the N-oxide, sulfinyl, or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy” refers to the group —O-(substituted heterocycyl).

“Heterocyclylthio” refers to the group —S-heterocycyl.

“Substituted heterocyclylthio” refers to the group —S-(substituted heterocycyl).

Examples of heterocycle and heteroaryls include, but are not limited to, azetidine, pyrrole, furan, thiophene, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O).

Phenylene refers to a divalent aryl ring, where the ring contains 6 carbon atoms.

Substituted phenylene refers to phenylenes which are substituted with 1 to 4, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Spirocycloalkyl” and “spiro ring systems” refers to divalent cyclic groups from 3 to 10 carbon atoms having a cycloalkyl or heterocycloalkyl ring with a spiro union (the union formed by a single atom which is the only common member of the rings) as exemplified by the following structure:

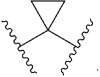 [SMILES: *C1(*)CC1]

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—.

“Substituted sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thioxo” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

“Optionally substituted” refers to a group selected from that group and a substituted form of that group. Substituted groups are defined herein. In one embodiment, subtituents are selected from C₁-C₁₀ or C₁-C₆ alkyl, C₂-C₆ alkenyl, C₂-C₆ alkynyl, C₆-C₁₀ aryl, C₃-C₈ cycloalkyl, C₂-C₁₀ heterocyclyl, C₁-C₁₀ heteroaryl, halo, —N₃, nitro, cyano, —CO₂H or a C₁-C₆ alkyl ester thereof.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

As used herein, the term stereochemically pure denotes a compound which has 80% or greater by weight of the indicated stereoisomer and 20% or less by weight of other stereoisomers. In a further embodiment, the compound of Formula (I), (II), or (III) has 90% or greater by weight of the stated stereoisomer and 10% or less by weight of other stereoisomers. In a yet further embodiment, the compound of Formula (I), (II), or (III) has 95% or greater by weight of the stated stereoisomer and 5% or less by weight of other stereoisomers. In a still further embodiment, the compound of formula (I), (II), or (III) has 97% or greater by weight of the stated stereoisomer and 3% or less by weight of other stereoisomers.

“Pharmaceutically acceptable salt” refers to salts of a compound, which salts are suitable for pharmaceutical use and are derived from a variety of organic and inorganic counter ions well known in the art and include, when the compound contains an acidic functionality, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate (see Stahl and Wermuth, eds., “Handbook of Pharmaceutically Acceptable Salts,” (2002), Verlag Helvetica Chimica Acta, Zirich, Switzerland), for a discussion of pharmaceutical salts, their selection, preparation, and use.

Generally, pharmaceutically acceptable salts are those salts that retain substantially one or more of the desired pharmacological activities of the parent compound and which are suitable for in vivo administration. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydroiodic acid, etc.), sulfuric acid, nitric acid, phosphoric acid, and the like.

Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), glutamic acid, hydroxynaphthoic acid, salicylic acid, stearic acid, muconic acid, and the like.

Pharmaceutically acceptable salts also include salts formed when an acidic proton present in the parent compound is either replaced by a metal ion (e.g., an alkali metal ion, an alkaline earth metal ion, or an aluminum ion) or by an ammonium ion (e.g., an ammonium ion derived from an organic base, such as, ethanolamine, diethanolamine, triethanolamine, morpholine, piperidine, dimethylamine, diethylamine, triethylamine, and ammonia).

A solvate of a compound is a solid-form of a compound that crystallizes with less than one, one or more than one molecules of a solvent inside in the crystal lattice. A few examples of solvents that can be used to create solvates, such as pharmaceutically acceptable solvates, include, but are not limited to, water, C₁-C₆ alcohols (such as methanol, ethanol, isopropanol, butanol, and can be optionally substituted) in general, tetrahydrofuran, acetone, ethylene glycol, propylene glycol, acetic acid, formic acid, and solvent mixtures thereof. Other such biocompatible solvents which may aid in making a pharmaceutically acceptable solvate are well known in the art. Additionally, various organic and inorganic acids and bases can be added to create a desired solvate. Such acids and bases are known in the art. When the solvent is water, the solvate can be referred to as a hydrate. In some embodiments, one molecule of a compound can form a solvate with from 0.1 to 5 molecules of a solvent, such as 0.5 molecules of a solvent (hemisolvate, such as hemihydrate), one molecule of a solvent (monosolvate, such as monohydrate) and 2 molecules of a solvent (disolvate, such as dihydrate).

An “effective amount” is an amount sufficient to effect beneficial or desired results. An effective amount can be administered in one or more administrations, applications or dosages and is determined by the system in which the drug or compound is delivered, e.g., an effective amount for in vitro purposes is not the same as an effective amount for in vivo purposes. For in vivo purposes, the delivery and “effective amount” is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

As used herein, “treating” or “treatment” of a disease in a patient refers to (1) preventing the symptoms or disease from occurring in an animal that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein, the term “polypeptide” refers to cell permeable peptides that can cross the cell membrane. Non-limiting examples of polypeptides include cationic polypeptides having from about 3 to about 30 amino acids having 5 or more positively charged amino acids, e.g., independently one or more of arginine or lysine. Other examples include: NH₂—RRRRRRRRR—COOH, NH₂—YGRKKRRQRRR—COOH, NH₂-TRSSRAGLQFPVGRVHRLLRK—COOH, NH₂-YTIWMPENPRPGTPCDIFTNSRGKRASNGGGGRRRRRR—COH, NH₂-GRKKRRQRRRPPQ-COOH, NH₂—WEAKLAKALAKALAKHLAKALAKALKACEA-COOH, NH₂—INLKALAALAKKI—COOH, NH₂—RQIKIWFQNRRMKWKKGG-COOH, NH₂—KETWWETWWTEWSQPKKKRKV—COOH, NH₂-KETWWETWWTEWSQPKKKRKV—COOH, and NH₂-YTIWMPENPRPGTPCDIFTNSRGKRASNG-COOH. In some embodiments, the polypeptide is represented by the variable P. In some embodiments, the polypeptide is attached to the carbonyl via its N-terminus. In some embodiments, the polypeptide is a lysine and/or arginine rich polypeptide. In some embodiments the polypeptide comprises 9-30 amino acid residues. Other cell permeable polypeptides that can cross the cell membrane are well-known in the art.

However, the proteins and polypeptides as used herein are not limited to human-derived proteins but may have an amino acid sequence derived from other animals, particularly, a warm-blooded animal (e.g., rat, guinea pig, mouse, chicken, rabbit, pig, sheep, cow, monkey, etc.).

“Poly adenosine diphosphate ribose (ADP) transferase activity” intends the activity of Poly-(ADP-ribose) polymerases (PARPs) that are found mostly in eukaryotes and catalyze the transfer of multiple ADP-ribose molecules to target proteins. As with mono-ADP ribosylation, the source of ADP-ribose is NAD⁺. PARPs use a catalytic triad of His-Tyr-Glu to facilitate binding of NAD⁺ and positioning of the end of the existing poly-ADP ribose chain on the target protein; the Glu facilitates catalysis and formation of a (1->2)O-glycosidic linkage between two ribose molecules. There are several other enzymes that recognize poly-ADP ribose chains, hydrolyse them or form branches.

“Adenosine diphosphate ribose (ADP) ribosyltransferase activity” intends the intracellular action of the addition of one or more ADP-ribose moieties to a protein. It is a reversible post-translational modification that is involved in many cellular processes, including cell signaling, DNA repair, gene regulation and apoptosis. Improper ADP-ribosylation has been implicated in some forms of cancer.

“Nicotinamide adenine dinucleotide (NAD⁺) (also known as diphosphopyridine nucleotide (DPN+) and Coenzyme I) intends the coenzyme found in all cells. The compound is a dinucleotide, and it consists of two nucleotides joined through their phosphate groups. groups. The chemical structure is provide below:

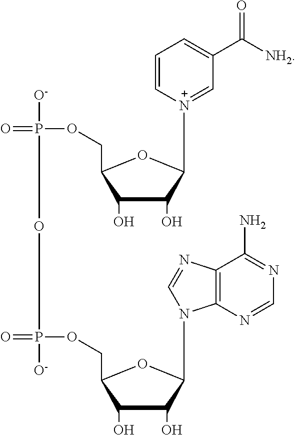

A “signal reagent” intends an agent (chemical, biological or otherwise) that emits a detectable signal.

The term “ADP-ribosyltransferase inhibitor” intends a molecule or an agent that inhibits the activity of ADP-ribosyltransferease.

Poly (ADP-ribose) polymerase (PARP) is a family of proteins involved in a number of cellular processes such as DNA repair, genomic stability, and programmed cell death. The PARP family comprises 17 members. PARP is composed of four domains of interest: a DNA-binding domain, a caspase-cleaved domain, an auto-modification domain, and a catalytic domain. The DNA-binding domain is composed of two zinc finder motifs. In the presence of damaged DNA (base pair-excised), the DNA-binding domain will bind the DNA and induce a conformational shift. It has been hypothesized that this binding occurs independent of the other domains. The auto-modification domain is responsible for releasing the protein from the DNA after catalysis. As used herein “PARP” intends means all different PARP isoforms (>15) from human genome, such as PARP1, 2, 3, 4, 5A, 5B, 10, 14, 15, 16, etc. “Under conditions that favor a PARP catalyzed reaction” intends suitable temperature, salt and necessary co-factors for PARP to act on a substrate. Such conditions are known in the art, see, e.g., Jiang et al. (2010) J. Am. Chem. Soc. 132(27):9363-9372, and described herein.

As used herein, the term “detectable label” intends a directly or indirectly detectable compound or composition that is conjugated directly or indirectly to the composition to be detected, e.g., N-terminal histadine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, luminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

“Affinity label” as used herein refers to a compound, that may be appended to a protein or another compound so that the protein or other compound can be purified from its crude source using an affinity purification technique, for example affinity chromatography, wherein the purification processes selects for the affinity label and the protein or other compound appened thereto based on the label's interactions with an affinity matrix used for the purification. These interactions include, but are not limited to, antigen-antibody interactions, enzyme-substrate interactions, receptor-ligand interactions, hydrogen bonding, ionic interactions or electrostatic interactions. Non-limiting examples of affinity labels include chitin binding protein (CBP), maltose binding protein (MBP), Strep-tag, glutathione-S-transferase (GST), poly(His) tags, NE-tag, Spot-tag, albumin-binding protein (ABP), alkaline phosphatase (AP), AU epitopes, bacteriophage T7 or V5 epitope, HSV epitope, biotin-carboxy carrier protein, biotin, and bluetounge virus tag (B-tag). Non limiting examples of matrices include, but are not limited to, albumin/low pH, mAb/low pH, avidin or streptavidin/biotin or denaturation, calmodulin/EGTA or EGTA and high salt, chloramphenicol/chloramphenicol, chitin, choline, methotrexate/folate, galactose, glutathione, and a divalent metal.

As used herein, the term “contacting” intends bringing the reagents into close proximity with each other so that a chemical or biochemical reaction can occur among the reagents. In one aspect, the term intends admixing the components, either in a reaction vessel or on a plate or dish. In another aspect, it intends in vivo administration to a subject.

The term “binding” or “binds” as used herein are meant to include interactions between molecules that may be covalent or non-covalent which, in one embodiment, can be detected using, for example, a hybridization assay. The terms are also meant to include “binding” interactions between molecules. Interactions may be, for example, protein-protein, antibody-protein, protein-nucleic acid, protein-small molecule or small molecule-nucleic acid in nature. This binding can result in the formation of a “complex” comprising the interacting molecules. A “complex” refers to the binding of two or more molecules held together by covalent or non-covalent bonds, interactions or forces.

The term “polypeptide” is used interchangeably with the term “protein” and in its broadest sense refers to a compound of two or more subunit amino acids, amino acid analogs or peptidomimetics. The subunits may be linked by peptide bonds. In another embodiment, the subunit may be linked by other bonds, e.g., ester, ether, etc. As used herein the term “amino acid” refers to natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein. The term “peptide fragment,” as used herein, also refers to a peptide chain.

It is to be inferred without explicit recitation and unless otherwise intended, that when the present invention relates to a polypeptide, protein, polynucleotide or antibody, an equivalent or a biologically equivalent of such is intended within the scope of this invention. As used herein, the term “biological equivalent thereof” is intended to be synonymous with “equivalent thereof” when referring to a reference protein, antibody, fragment, polypeptide or nucleic acid, intends those having minimal homology while still maintaining desired structure or functionality. Unless specifically recited herein, it is contemplated that any polynucleotide, polypeptide or protein mentioned herein also includes equivalents thereof. In one aspect, an equivalent polynucleotide is one that hybridizes under stringent conditions to the polynucleotide or complement of the polynucleotide as described herein for use in the described methods. In another asect, an equivalent antibody or antigen binding polypeptide intends one that binds with at least 70%, or alternatively at least 75%, or alternatively at least 80%, or alternatively at least 85%, or alternatively at least 90%, or alternatively at least 95% affinity or higher affinity to a reference antibody or antigen binding fragment. In another aspect, the equivalent thereof competes with the binding of the antibody or antigen binding fragment to its antigen under a competitive ELISA assay. In another aspect, an equivalent intends at least about 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid.

“Homology” or “identity” or “similarity” are synonymously and refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

### Modes for Carrying Out the Disclosure

Provided herein are novel molecular tools to study protein ADP-ribosylation in live cells. The compounds provided herein can be in situ converted to NAD⁺ analogues as universal ART cofactors and will thus provide invaluable and generally applicable tools for non-invasive monitoring and tracking of global ADP-ribosylation with striking spatiotemporal resolution (FIG. 1). These novel chemical tools can be applied virtually to any types of primary or established cells and even organisms for in vitro and in vivo functionally exploring ADP-ribosylation across entire ART superfamily.

### Compounds

In one aspect, provided herein is a compound of Formula (I-A):

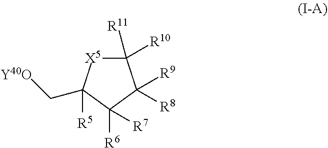

or a tautomer thereof, or an N-oxide of each thereof, or a pharmaceutically acceptable salt of each of the aforementioned, or a pharmaceutically acceptable solvate of each of the foregoing, wherein: each R⁵, R⁶, R⁷, R⁸, R⁹, R¹⁰, and R¹¹ independently is a hydrogen, —N₃, a hydroxyl, an optionally substituted C₁-C₁₀ alkyl, an optionally substituted C₂-C₁₀ alkynyl, an optionally substituted C₁-C₁₀ alkoxy, —SR³⁰, an optionally substituted C₆-C₁₀ aryl, an optionally substituted 5-15 membered heteroaryl, or Z; X⁵ is —S—, —O—, or —NR²⁰—; each R²⁰ and R³⁰ is independently a hydrogen or an optionally substituted C₁-C₁₀ alkyl; Z is

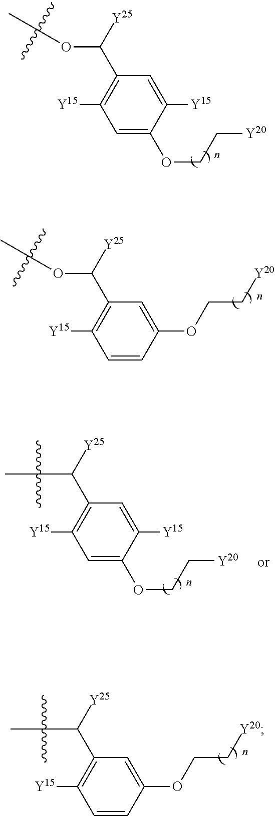

each n is independently 1, 2, 3 or 4 or 1-4; each Y¹⁵ is independently a hydrogen, —NO₂, a halo, a cyano, a hydroxyl, an optionally substituted C₁-C₆ alkyl, or an optionally substituted C₁-C₆ alkoxy; each Y²⁵ is independently a hydrogen or an optionally substituted C₁-C₆ alkyl, an each Y²⁰ is independently selected from the group consisting of:

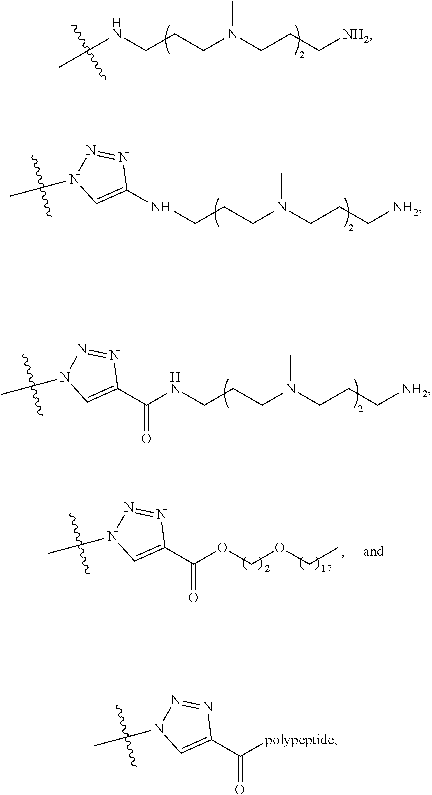

Y⁴⁰ is a hydrogen, an optionally substituted C₁-C₆ alkyl, an optionally substituted C₂-C₁₀ alkenyl, an optionally substituted C₂-C₁₀ alkynyl, an optionally substituted C₆-C₁₀ aryl, an optionally substituted 5-15 membered heteroaryl, or -L¹Y³⁵; L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—; each R¹⁰⁰ is independently —O^{⊖}, an optionally substituted C₁-C₁₀ alkyl group, or an optionally substituted C₁-C₁₀ alkoxy; and Y³⁵ is a hydroxyl or an optionally substituted C₁-C₆ alkoxy, and polypeptide is a cationic polypeptide having about 5 to 30 amino acid residues.

In some embodiments, each n is independently 1, 3, or 4.

In one aspect, provided herein is a compound of Formula (I-A):

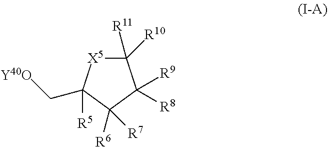

or a tautomer thereof, or an N-oxide of each thereof, or a pharmaceutically acceptable salt of each of the aforementioned, or a pharmaceutically acceptable solvate of each of the foregoing, wherein: each R⁵, R⁶, R⁷, R⁸, R⁹, R¹⁰, and R¹¹ independently is a hydrogen, —N₃, a hydroxyl, an optionally substituted C₁-C₁₀ alkyl, an optionally substituted C₂-C₁₀ alkynyl, an optionally substituted C₁-C₁₀ alkoxy, —SR³⁰, an optionally substituted C₆-C₁₀ aryl, an optionally substituted 5-15 membered heteroaryl, or Z; X⁵ is —S—, —O—, or —NR²⁰—; each R²⁰ and R³⁰ is independently a hydrogen or an optionally substituted C₁-C₁₀ alkyl; Z is

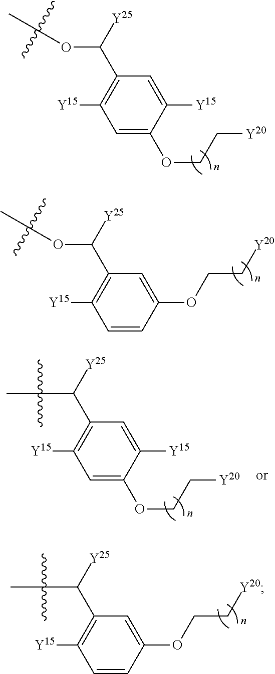

each n is independently 1, 2, 3 or 4; each Y¹⁵ is independently a hydrogen, —NO₂, a halo, a cyano, a hydroxyl, an optionally substituted C₁-C₆ alkyl, or an optionally substituted C₁-C₆ alkoxy; each Y²⁵ is independently a hydrogen or an optionally substituted C₁-C₆ alkyl, an each Y²⁰ is independently selected from the group consisting of:

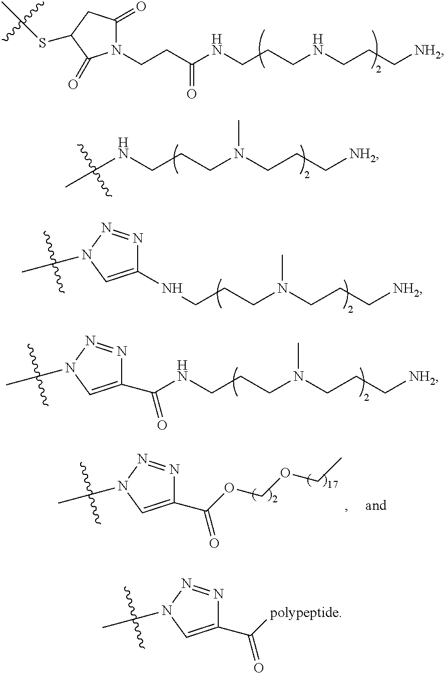

Y⁴⁰ is a hydrogen, an optionally substituted C₁-C₆ alkyl, an optionally substituted C₂-C₁₀ alkenyl, an optionally substituted C₂-C₁₀ alkynyl, an optionally substituted C₆-C₁₀ aryl, an optionally substituted 5-15 membered heteroaryl, or -L¹Y³⁵; L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)each R¹⁰⁰ is independently —O^{⊖}, an optionally substituted C₁-C₁₀ alkyl group, or an optionally substituted C₁-C₁₀ alkoxy; and Y³⁵ is a hydroxyl or an optionally substituted C₁-C₆ alkoxy; and and polypeptide is a cationic polypeptide having about 5 to 30 amino acid residues

In some embodiments, each n is independently 1, 3, or 4.

In some embodiments, the compound of Formula (I-A) is of Formula (I-AA):

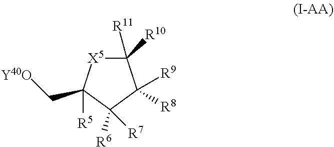

or a tautomer thereof, or an N-oxide of each thereof, or a pharmaceutically acceptable salt of each of the aforementioned, or a pharmaceutically acceptable solvate of each of the foregoing, wherein the variables are as defined as in any of the above embodiments.

In one aspect, provided herein is a compound of Formula (I-B):

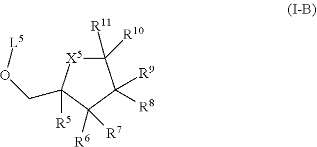

or a tautomer thereof, or an N-oxide of each thereof, or a pharmaceutically acceptable salt of each of the aforementioned, or a pharmaceutically acceptable solvate of each of the foregoing, wherein: each R⁵, R⁶, R⁷, R⁸, R⁹, R¹⁰, and R¹¹ independently is a hydrogen, —N₃, a hydroxyl, an optionally substituted C₁-C₁₀ alkyl, an optionally substituted C₂-C₁₀ alkynyl, an optionally substituted C₁-C₁₀ alkoxy, —SR³⁰, an optionally substituted C₆-C₁₀ aryl, an optionally substituted 5-15 membered heteroaryl, or Z; X⁵ is —S—, —O—, or —NR²⁰—; each R²⁰ and R³⁰ is independently a hydrogen or an optionally substituted C₁-C₁₀ alkyl; Z is

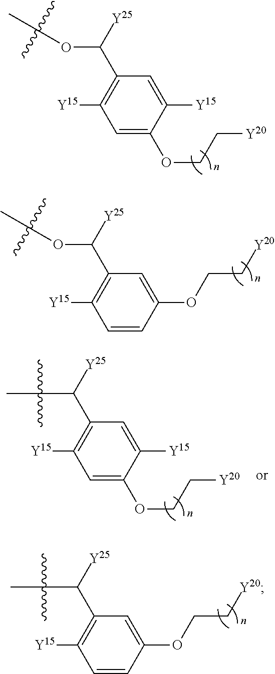

each n is independently 1, 2, 3, or 4; each Y¹⁵ is independently a hydrogen, —NO₂, a halo, a cyano, a hydroxyl, an optionally substituted C₁-C₆ alkyl, or an optionally substituted C₁-C₆ alkoxy; each Y²⁵ is independently a hydrogen or an optionally substituted C₁-C₆ alkyl, and each Y²⁰ is independently selected from the group consisting of:

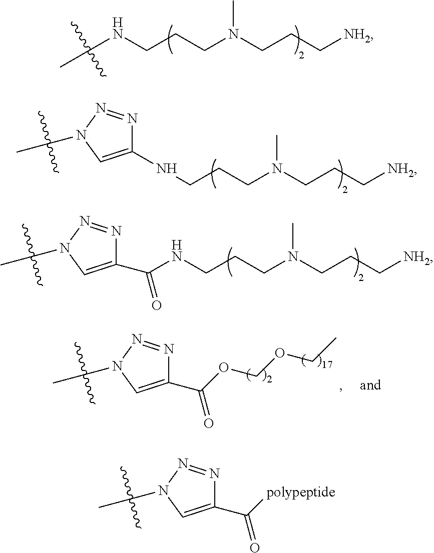

L⁵ is a hydrogen, an optionally substituted C₁-C₆ alkyl, or -L¹Y³⁵; L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—; each R¹⁰⁰ is independently —O^{⊖}, an optionally substituted C₁-C₁₀ alkyl group, or an optionally substituted C₁-C₁₀ alkoxy; and Y³⁵ is a hydroxyl or an optionally substituted C₁-C₆ alkoxy, and polypeptide is a cationic polypeptide having about 5 to 30 amino acid residues.

In some embodiments, each n is independently 1, 3, or 4.

In one aspect, provided herein is a compound of Formula (I-B):

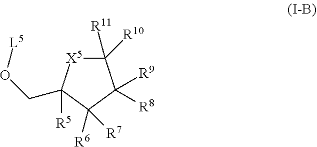

or a tautomer thereof, or an N-oxide of each thereof, or a pharmaceutically acceptable salt of each of the aforementioned, or a pharmaceutically acceptable solvate of each of the foregoing, wherein: each R⁵, R⁶, R⁷, R⁸, R⁹, R¹⁰, and R¹¹ independently is a hydrogen, —N₃, a hydroxyl, an optionally substituted C₁-C₁₀ alkyl, an optionally substituted C₂-C₁₀ alkynyl, an optionally substituted C₁-C₁₀ alkoxy, —SR³⁰, an optionally substituted C₆-C₁₀ aryl, an optionally substituted 5-15 membered heteroaryl, or Z; X⁵ is —S—, —O—, or —NR²⁰—; each R²⁰ and R³⁰ is independently a hydrogen or an optionally substituted C₁-C₁₀ alkyl; Z is

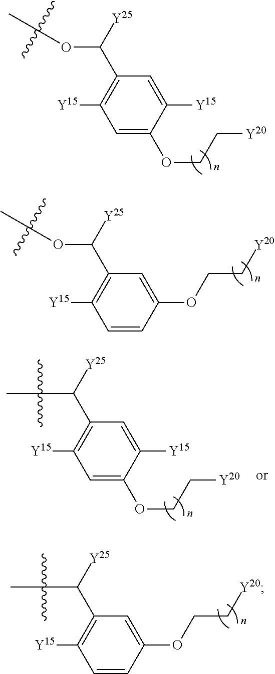

each n is independently 1, 2, 3, or 4; each Y¹⁵ is independently a hydrogen, —NO₂, a halo, a cyano, a hydroxyl, an optionally substituted C₁-C₆ alkyl, or an optionally substituted C₁-C₆ alkoxy; each Y²⁵ is independently a hydrogen or an optionally substituted C₁-C₆ alkyl, and each Y²⁰ is independently selected from the group consisting of:

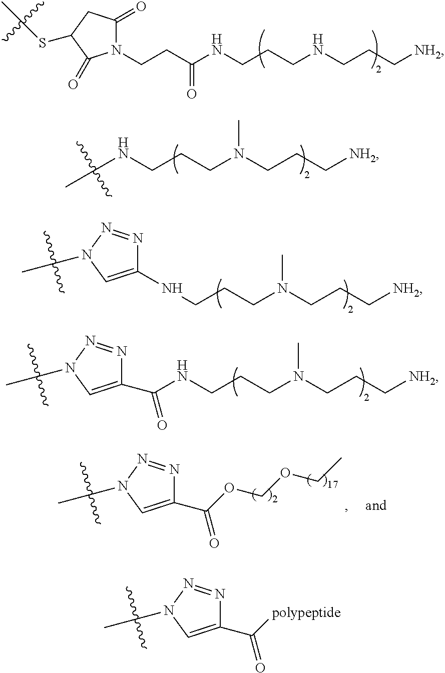

L⁵ is a hydrogen, an optionally substituted C₁-C₆ alkyl, or -L¹Y³⁵; L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—; each R¹⁰⁰ is independently —O^{⊖}, an optionally substituted C₁-C₁₀ alkyl group, or an optionally substituted C₁-C₁₀ alkoxy; and Y³⁵ is a hydroxyl or an optionally substituted C₁-C₆ alkoxy and polypeptide is a cationic polypeptide having about 5 to 30 amino acid residues.

In some embodiments, each n is independently 1, 3, or 4.

In some embodiments, the compound of Formula (I-B) is of Formula (I-BB):

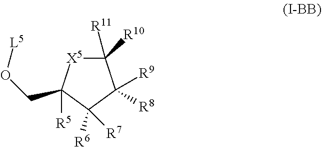

or a tautomer thereof, or an N-oxide of each thereof, or a pharmaceutically acceptable salt of each of the aforementioned, or a pharmaceutically acceptable solvate of each of the foregoing, wherein the variables are as defined in any of the embodiments above.

In one aspect, provided herein is a compound of Formula (I-C):

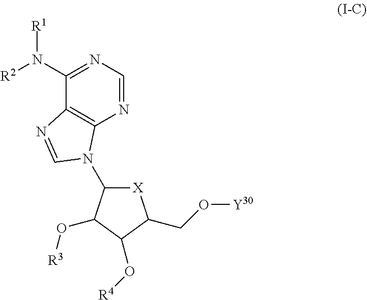

or a tautomer thereof, or an N-oxide of each thereof, or a pharmaceutically acceptable salt of each of the aforementioned, or a pharmaceutically acceptable solvate of each of the foregoing, wherein: each R¹, R², R³, and R⁴ independently is a hydrogen or an optionally substituted C₁-C₆ alkyl or Z; X is —S—, —O—, or —NR²⁰—; R²⁰ is a hydrogen or an optionally substituted C₁-C₁₀ alkyl; Z is

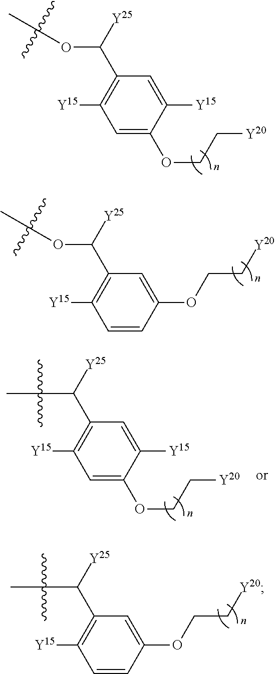

each n is independently 1, 2, 3, or 4; each Y¹⁵ is independently a hydrogen, —NO₂, a halo, a cyano, a hydroxyl, an optionally substituted C₁-C₆ alkyl, or an optionally substituted C₁-C₆ alkoxy; each Y²⁵ is independently a hydrogen or an optionally substituted C₁-C₆ alkyl, and each Y²⁰ is independently selected from the group consisting of:

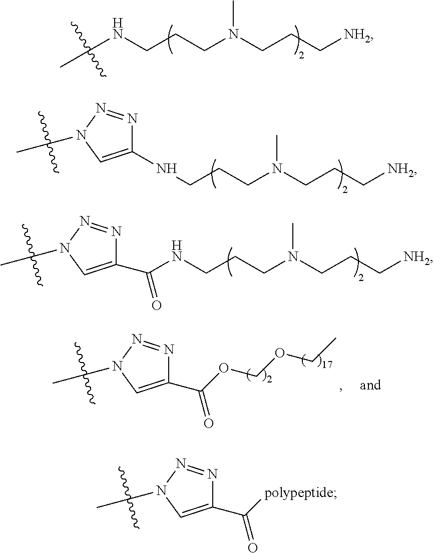

Y³⁰ is a hydrogen, an optionally substituted C₁-C₆ alkyl, an optionally substituted C₂-C₁₀ alkenyl, an optionally substituted C₂-C₁₀ alkynyl, an optionally substituted C₆-C₁₀ aryl, an optionally substituted 5-15 membered heteroaryl, or -L¹Y³⁵; L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—; each R¹⁰⁰ is independently —O^{⊖}, an optionally substituted C₁-C₁₀ alkyl group, or an optionally substituted C₁-C₁₀ alkoxy; and Y³⁵ is a hydroxyl or an optionally substituted C₁-C₆ alkoxy, and polypeptide is a cationic polypeptide having about 5 to 30 amino acid residues.

In some embodiments, each n is independently 1, 3, or 4.

In one aspect, provided herein is a compound of Formula (I-C):

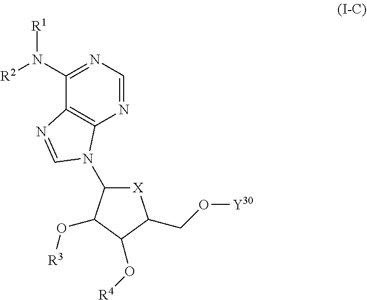

or a tautomer thereof, or an N-oxide of each thereof, or a pharmaceutically acceptable salt of each of the aforementioned, or a pharmaceutically acceptable solvate of each of the foregoing, wherein: each R¹, R², R³, and R⁴ independently is a hydrogen or an optionally substituted C₁-C₆ alkyl or Z; X is —S—, —O—, or —NR²⁰—; R²⁰ is a hydrogen or an optionally substituted C₁-C₁₀ alkyl;

### Z is

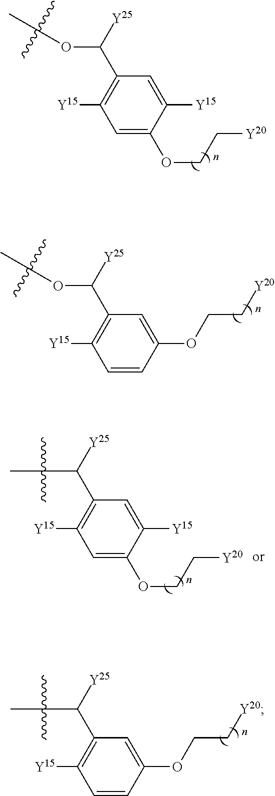

each n is independently 1, 2, 3, or 4; each Y¹⁵ is independently a hydrogen, —NO₂, a halo, a cyano, a hydroxyl, an optionally substituted C₁-C₆ alkyl, or an optionally substituted C₁-C₆ alkoxy; each Y²⁵ is independently a hydrogen or an optionally substituted C₁-C₆ alkyl, and each Y²⁰ is independently selected from the group consisting of:

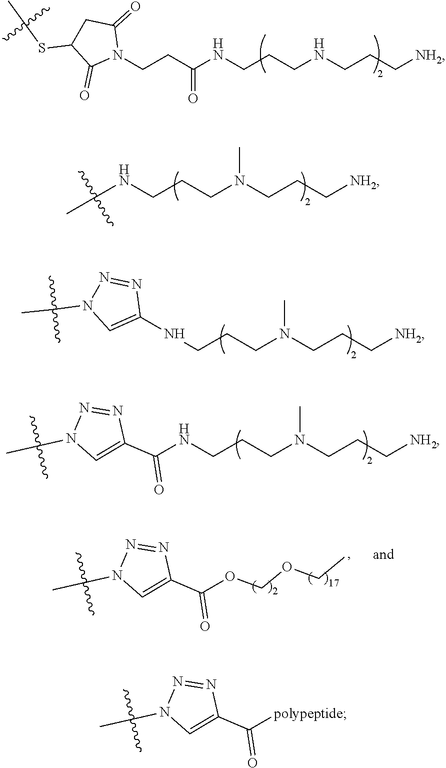

Y³⁰ is a hydrogen, an optionally substituted C₁-C₆ alkyl, an optionally substituted C₂-C₁₀ alkenyl, an optionally substituted C₂-C₁₀ alkynyl, an optionally substituted C₆-C₁₀ aryl, an optionally substituted 5-15 membered heteroaryl, or -L¹Y³⁵; L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—; each R¹⁰⁰ is independently —O^{⊖}, an optionally substituted C₁-C₁₀ alkyl group, or an optionally substituted C₁-C₁₀ alkoxy; Y³⁵ is a hydroxyl or an optionally substituted C₁-C₆ alkoxy and polypeptide is a cationic polypeptide having about 5 to 30 amino acid residues.

In some embodiments, each n is independently 1, 3, or 4.

In some embodiments, the compound of Formula (I-C) is of Formula (I-CC):

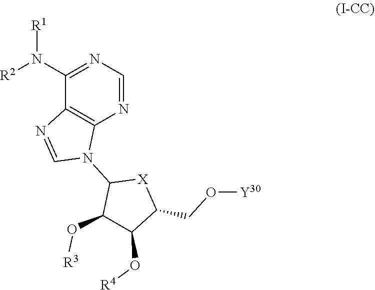

or a tautomer thereof, or an N-oxide of each thereof, or a pharmaceutically acceptable salt of each of the aforementioned, or a pharmaceutically acceptable solvate of each of the foregoing, wherein the variables are as defined as in any of the embodiments above.

In one aspect, provided herein is a compound of Formula (I-D):

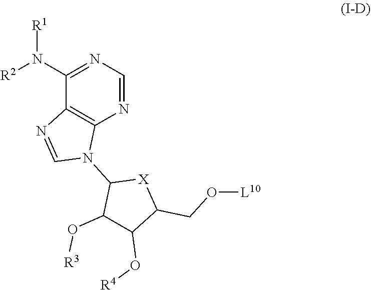

or a tautomer thereof, or an N-oxide of each thereof, or a pharmaceutically acceptable salt of each of the aforementioned, or a pharmaceutically acceptable solvate of each of the foregoing, wherein: each R¹, R², R³, and R⁴ independently is a hydrogen or an optionally substituted C₁-C₆ alkyl or Z; X is —S—, —O—, or —NR²⁰—; R²⁰ is a hydrogen or an optionally substituted C₁-C₁₀ alkyl;
L¹⁰ is a hydrogen, an optionally substituted C₁-C₆ alkyl, or -L¹Y³⁵; L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—; each R¹⁰⁰ is independently —O^{⊖}, an optionally substituted C₁-C₁₀ alkyl group, or an optionally substituted C₁-C₁₀ alkoxy; Y³⁵ is a hydroxyl or an optionally substituted C₁-C₆ alkoxy, and polypeptide is a cationic polypeptide having about 5 to 30 amino acid residues;

Z is

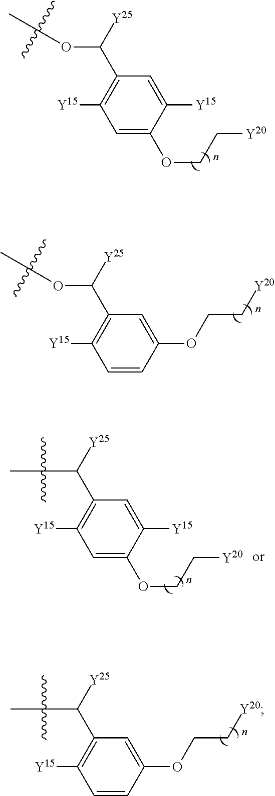

each n is independently 1, 2, 3, or 4;

each Y¹⁵ is independently a hydrogen, —NO₂, a halo, a cyano, a hydroxyl, an optionally substituted C₁-C₆ alkyl, or an optionally substituted C₁-C₆ alkoxy;

each Y²⁵ is independently a hydrogen or an optionally substituted C₁-C₆ alkyl, and

each Y²⁰ is independently selected from the group consisting of:

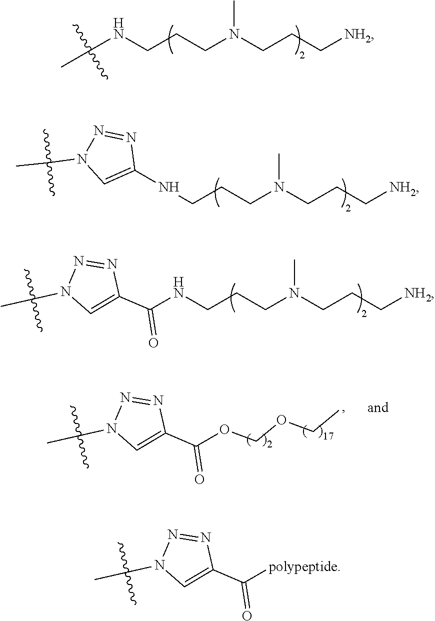

In some embodiments, each n is independently 1, 2, 3, or 4.

In one aspect, provided herein is a compound of Formula (I-D):

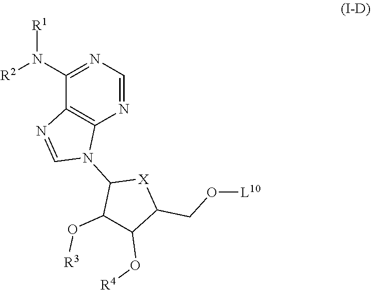

or a tautomer thereof, or an N-oxide of each thereof, or a pharmaceutically acceptable salt of each of the aforementioned, or a pharmaceutically acceptable solvate of each of the foregoing, wherein: each R¹, R², R³, and R⁴ independently is a hydrogen or an optionally substituted C₁-C₆ alkyl or Z; X is —S—, —O—, or —NR²⁰—; R²⁰ is a hydrogen or an optionally substituted C₁-C₁₀ alkyl; L¹⁰ is a hydrogen, an optionally substituted C₁-C₆ alkyl, or -L¹Y³⁵;

L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—; each R¹⁰⁰ is independently —O^{⊖}, an optionally substituted C₁-C₁₀ alkyl group, or an optionally substituted C₁-C₁₀ alkoxy; Y³⁵ is a hydroxyl or an optionally substituted C₁-C₆ alkoxy.

Z is

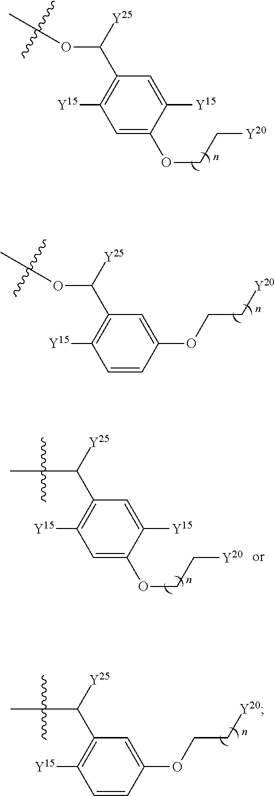

each n is independently 1, 2, 3, or 4; each Y¹⁵ is independently a hydrogen, —NO₂, a halo, a cyano, a hydroxyl, an optionally substituted C₁-C₆ alkyl, or an optionally substituted C₁-C₆ alkoxy; each Y²⁵ is independently a hydrogen or an optionally substituted C₁-C₆ alkyl, and each Y²⁰ is independently selected from the group consisting of:

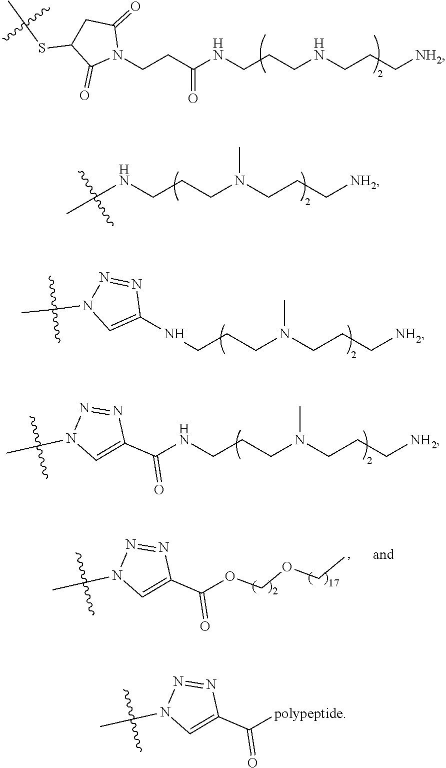

In some embodiments, each n is independently 1, 2, 3, or 4.

In some embodiments, the compound of Formula (I-D) is of Formula (I-DD):

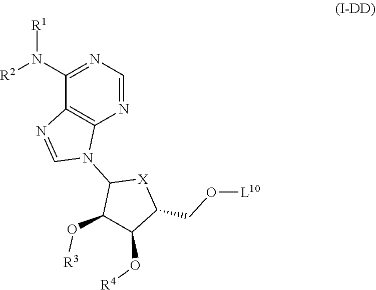

or a tautomer thereof, or an N-oxide of each thereof, or a pharmaceutically acceptable salt of each of the aforementioned, or a pharmaceutically acceptable solvate of each of the foregoing, wherein the variables are as defined in any of the embodiments above.

In one aspect, provided herein is a compound of Formula (I):

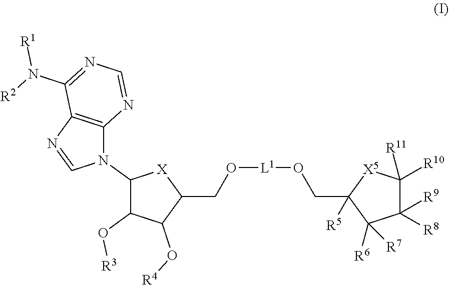

or a tautomer thereof, or an N-oxide of each thereof, or a pharmaceutically acceptable salt of each of the aforementioned, or a pharmaceutically acceptable solvate of each of the foregoing, wherein: each R¹, R², R³, and R⁴ independently is a hydrogen or an optionally substituted C₁-C₆ alkyl or Z; X is —S—, —O—, or —NR²⁰—; X⁵ is —S—, —O—, or —NR²⁰—; L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—; R¹⁰⁰ is —O^{⊖}, an optionally substituted C₁-C₁₀ alkyl group, or an optionally substituted C₁-C₁₀ alkoxy; each R⁵, R⁶, R⁷, R⁸, R⁹, R¹⁰, and R¹¹ independently is a hydrogen, —N₃, a hydroxyl, an optionally substituted C₁-C₁₀ alkyl, an optionally substituted C₂-C₁₀ alkynyl, an optionally substituted C₁-C₁₀ alkoxy, —SR³⁰, an optionally substituted C₆-C₁₀ aryl, an optionally substituted 5-15 membered heteroaryl, or Z;

Z is

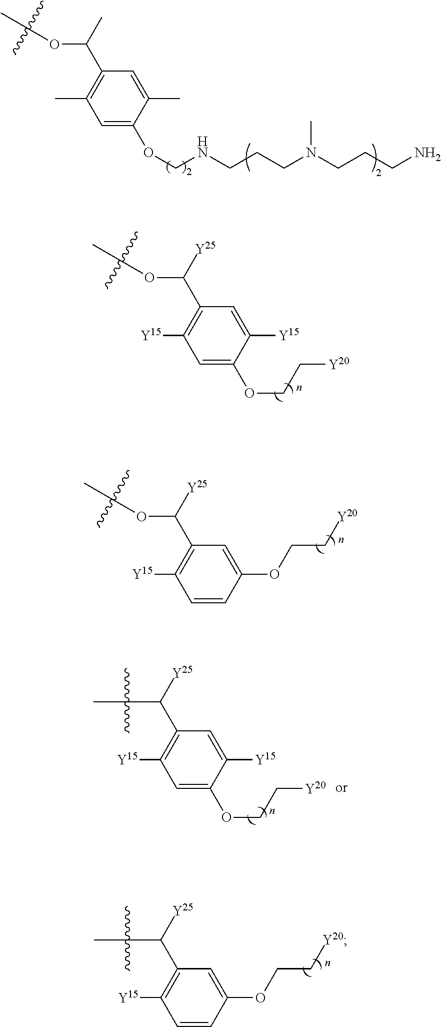

each n is independently 1, 2, 3, or 4; each Y¹⁵ is independently a hydrogen, a halo, a cyano, a hydroxyl, an optionally substituted C₁-C₆ alkyl, or an optionally substituted C₁-C₆ alkoxy; each Y²⁵ is independently a hydrogen or an optionally substituted C₁-C₆ alkyl, and each Y²⁰ is independently selected from the group consisting of:

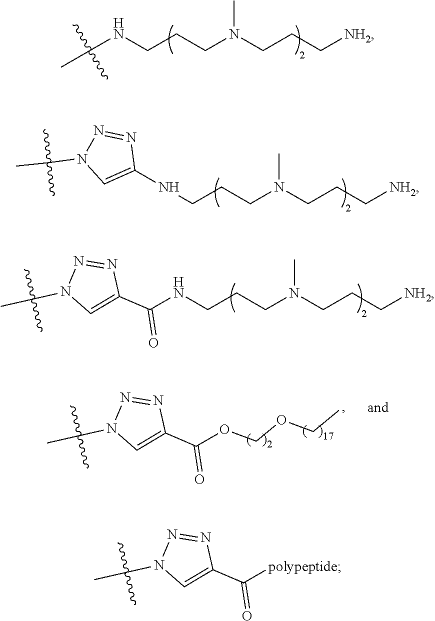

and

each R²⁰ and R³⁰ is independently a hydrogen or an optionally substituted C₁-C₁₀ alkyl and polypeptide is a cationic polypeptide having about 5 to 30 amino acid residues.

In some embodiments, each n is independently 1, 3, or 4.

In one aspect, provided herein is a compound of Formula (I):

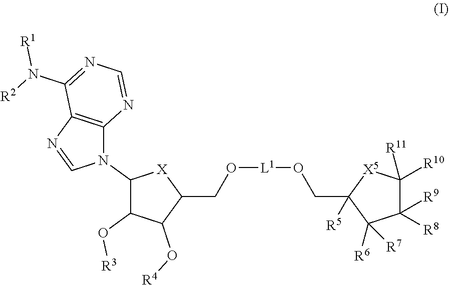

or a tautomer thereof, or an N-oxide of each thereof, or a pharmaceutically acceptable salt of each of the aforementioned, or a pharmaceutically acceptable solvate of each of the foregoing, wherein: each R¹, R², R³, and R⁴ independently is a hydrogen or an optionally substituted C₁-C₆ alkyl or Z; X is —S—, —O—, or —NR²⁰—; X⁵ is —S—, —O—, or —NR²⁰—; L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰); R¹⁰⁰ is —O^{⊖}, an optionally substituted C₁-C₁₀ alkyl group, or an optionally substituted C₁-C₁₀ alkoxy; each R⁵, R⁶, R⁷, R⁸, R⁹, R¹⁰, and R¹¹ independently is a hydrogen, —N₃, a hydroxyl, an optionally substituted C₁-C₁₀ alkyl, an optionally substituted C₂-C₁₀ alkynyl, an optionally substituted C₁-C₁₀ alkoxy, —SR³⁰, an optionally substituted C₆-C₁₀ aryl, an optionally substituted 5-15 membered heteroaryl, or Z;

Z is

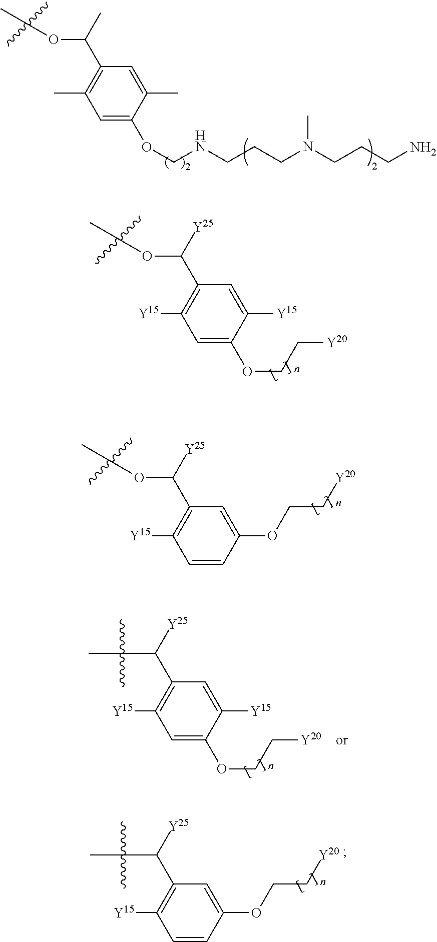

each n is independently 1, 2, 3, or 4; each Y¹⁵ is independently a hydrogen, a halo, a cyano, a hydroxyl, an optionally substituted C₁-C₆ alkyl, or an optionally substituted C₁-C₆ alkoxy; each Y²⁵ is independently a hydrogen or an optionally substituted C₁-C₆ alkyl, and each Y²⁰ is independently selected from the group consisting of:

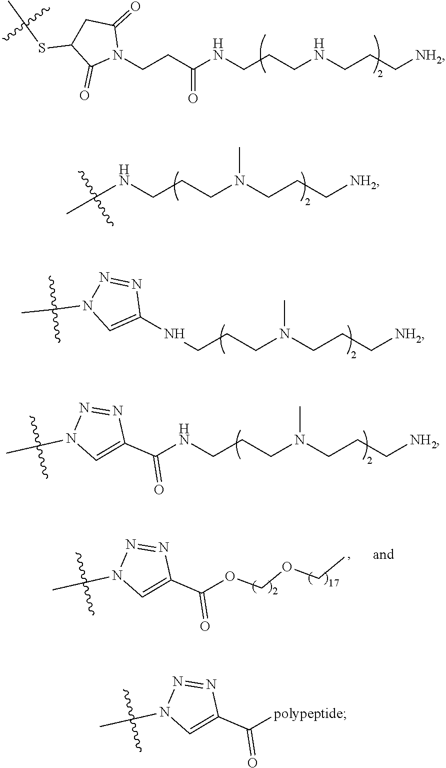

and

each R²⁰ and R³⁰ is independently a hydrogen or an optionally substituted C₁-C₁₀ alkyl and polypeptide is a cationic polypeptide having about 5 to 30 amino acid residues.

In some embodiments, each n is independently 1, 3, or 4.

In some embodiments, the compound of Formula (I) is of Formula (II):

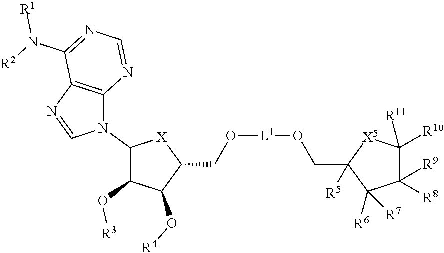

or a tautomer thereof, or an N-oxide of each thereof, or a pharmaceutically acceptable salt of each of the aforementioned, or a pharmaceutically acceptable solvate of each of the foregoing, wherein the variables are as defined in any of the above embodiments.

In some embodiments, the compound of Formula (I) is of Formula (III):

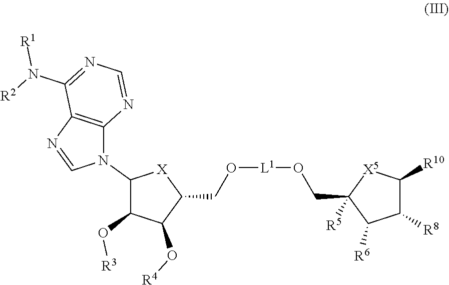

or a tautomer thereof, or an N-oxide of each thereof, or a pharmaceutically acceptable salt of each of the aforementioned, or a pharmaceutically acceptable solvate of each of the foregoing, wherein the variables are as defined in any of the above embodiments.

In some embodiments, R¹ is a hydrogen. In some embodiments, R¹ is an optionally substituted C₁-C₆ alkyl. In some embodiments, R¹ is Z.

In some embodiments, R² is a hydrogen. In some embodiments, R² is an optionally substituted C₁-C₆ alkyl. In some embodiments, R² is Z.

In some embodiments, R³ is a hydrogen. In some embodiments, R³ is an optionally substituted C₁-C₆ alkyl. In some embodiments, R³ is Z.

In some embodiments, R⁴ is a hydrogen. In some embodiments, R⁴ is an optionally substituted C₁-C₆ alkyl. In some embodiments, R⁴ is Z.

In some embodiments, X⁵ is —S—. In some embodiments, X⁵ is —O—. In some embodiments, X⁵ is —NR²⁰—.

In some embodiments, X⁵ is —S—. In some embodiments, X⁵ is —O—. In some embodiments, X⁵ is —NR²⁰—.

In some embodiments, L¹ is —PO₂—. In some embodiments, L¹ is —PO₃—PO₂—. In some embodiments, L¹ is —PO₃—PO₃—PO₂—. In some embodiments, L¹ is —P(═O)(R¹⁰⁰)—. In some embodiments, L¹ is —P(═O)(R¹⁰⁰)OP(═O)(R¹⁰⁰)—. In some embodiments, L¹ is —P(═O)(R¹⁰⁰)OP(═O)(R¹⁰⁰)OP(═O)(R¹⁰⁰)—.

In some embodiments, R⁵ is a hydrogen. In some embodiments, R⁵ is —N₃. In some embodiments, R⁵ is a hydroxyl. In some embodiments, R⁵ is an optionally substituted C₁-C₁₀ alkyl. In some embodiments, R⁵ is an optionally substituted C₂-C₁₀ alkynyl. In some embodiments, R⁵ is an optionally substituted C₁-C₁₀ alkoxy. In some embodiments, R⁵ is —SR³⁰. In some embodiments, R⁵ is an optionally substituted C₆-C₁₀ aryl. In some embodiments, R⁵ is an optionally substituted 5-15 membered heteroaryl. In some embodiments, R⁵ is Z.

In some embodiments, R⁶ is a hydrogen. In some embodiments, R⁶ is —N₃. In some embodiments, R⁶ is a hydroxyl. In some embodiments, R⁶ is an optionally substituted C₁-C₁₀ alkyl. In some embodiments, R⁶ is an optionally substituted C₂-C₁₀ alkynyl. In some embodiments, R⁶ is an optionally substituted C₁-C₁₀ alkoxy. In some embodiments, R⁶ is —SR³⁰. In some embodiments, R⁶ is an optionally substituted C₆-C₁₀ aryl. In some embodiments, R⁶ is an optionally substituted 5-15 membered heteroaryl. In some embodiments, R⁶ is Z.

In some embodiments, R⁷ is a hydrogen. In some embodiments, R⁷ is —N₃. In some embodiments, R⁷ is a hydroxyl. In some embodiments, R⁷ is an optionally substituted C₁-C₁₀ alkyl. In some embodiments, R⁷ is an optionally substituted C₂-C₁₀ alkynyl. In some embodiments, R⁷ is an optionally substituted C₁-C₁₀ alkoxy. In some embodiments, R⁷ is —SR³⁰. In some embodiments, R⁷ is an optionally substituted C₆-C₁₀ aryl. In some embodiments, R⁷ is an optionally substituted 5-15 membered heteroaryl. In some embodiments, R⁷ is Z.

In some embodiments, R⁸ is a hydrogen. In some embodiments, R⁸ is —N₃. In some embodiments, R⁸ is a hydroxyl. In some embodiments, R⁸ is an optionally substituted C₁-C₁₀ alkyl. In some embodiments, R⁸ is an optionally substituted C₂-C₁₀ alkynyl. In some embodiments, R⁸ is an optionally substituted C₁-C₁₀ alkoxy. In some embodiments, R⁸ is —SR³⁰. In some embodiments, R⁸ is an optionally substituted C₆-C₁₀ aryl. In some embodiments, R⁸ is an optionally substituted 5-15 membered heteroaryl. In some embodiments, R⁸ is Z.

In some embodiments, R⁹ is a hydrogen. In some embodiments, R⁹ is —N₃. In some embodiments, R⁹ is a hydroxyl. In some embodiments, R⁹ is an optionally substituted C₁-C₁₀ alkyl. In some embodiments, R⁹ is an optionally substituted C₂-C₁₀ alkynyl. In some embodiments, R⁹ is an optionally substituted C₁-C₁₀ alkoxy. In some embodiments, R⁹ is —SR³⁰. In some embodiments, R⁹ is an optionally substituted C₆-C₁₀ aryl. In some embodiments, R⁹ is an optionally substituted 5-15 membered heteroaryl. In some embodiments, R⁹ is Z.

In some embodiments, R⁷, R⁹ and R¹¹ are each hydrogen.

In some embodiments, R¹⁰ is a hydrogen. In some embodiments, R¹⁰ is —N₃. In some embodiments, R¹⁰ is a hydroxyl. In some embodiments, R¹⁰ is an optionally substituted C₁-C₁₀ alkyl. In some embodiments, R¹⁰ is an optionally substituted C₂-C₁₀ alkynyl. In some embodiments, R¹⁰ is an optionally substituted C₁-C₁₀ alkoxy. In some embodiments, R¹⁰ is —SR³⁰. In some embodiments, R¹⁰ is an optionally substituted C₆-C₁₀ aryl. In some embodiments, R¹⁰ is an optionally substituted 5-15 membered heteroaryl. In some embodiments, R¹⁰ is Z.

In some embodiments, R¹¹ is a hydrogen. In some embodiments, R¹¹ is —N₃. In some embodiments, R¹¹ is a hydroxyl. In some embodiments, R¹¹ is an optionally substituted C₁-C₁₀ alkyl. In some embodiments, R¹ is an optionally substituted C₂-C₁₀ alkynyl. In some embodiments, R¹ is an optionally substituted C₁-C₁₀ alkoxy. In some embodiments, R¹ is —SR³⁰. In some embodiments, R¹¹ is an optionally substituted C₆-C₁₀ aryl. In some embodiments, R¹¹ is an optionally substituted 5-15 membered heteroaryl. In some embodiments, R¹¹ is Z.

In some embodiments, R¹⁰⁰ is —O^{⊖}. In some embodiments, R¹⁰⁰ is an optionally substituted C₁-C₁₀ alkyl group. In some embodiments, R¹⁰⁰ is an optionally substituted C₁-C₁₀ alkoxy. In some embodiments, R¹⁰⁰ is a methyl. In some embodiments, R¹⁰⁰ is a methoxy. In some embodiments, R¹⁰⁰ is a C₁-C₁₀ alkyl group optionally substituted with a C₂ alkynyl. In some embodiments, R¹⁰⁰ is a C₁-C₆ alkyl group optionally substituted with a C₂ alkynyl. In some embodiments, R¹⁰⁰ is

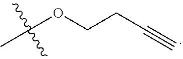 [SMILES: *OCCC#C]

In some embodiments, R¹⁰⁰ is selected from the group consisting of:

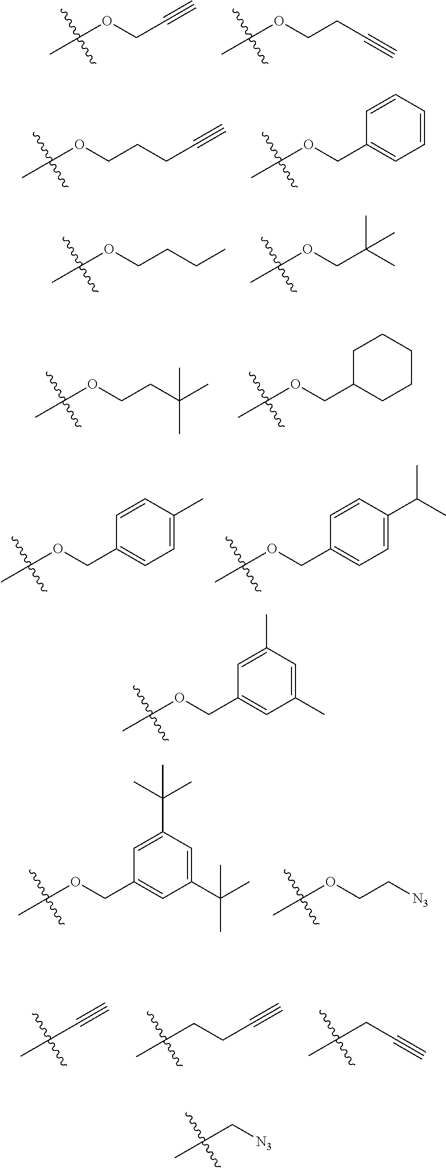

In some embodiments, R¹⁰⁰ is a methyl. In some embodiments, R¹⁰⁰ is an optionally substituted methyl. In some embodiments, R¹⁰⁰ is a methoxy. In some embodiments, R¹⁰⁰ is an optionally substituted methoxy.

In some embodiments, R²⁰ is a hydrogen. In some embodiments, R²⁰ is an optionally substituted C₁-C₁₀ alkyl.

In some embodiments, R³⁰ is a hydrogen. In some embodiments, R³⁰ is an optionally substituted C₁-C₁₀ alkyl.

In some embodiments, Z is

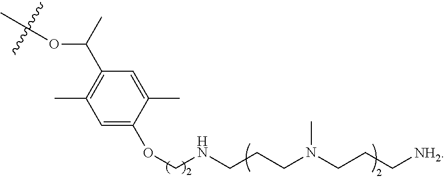

In some embodiments, Z is

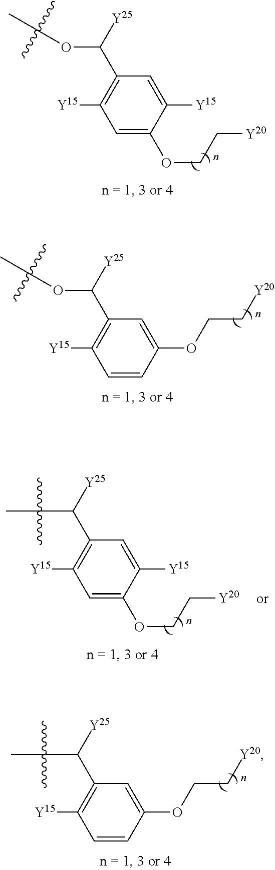

Wherein: each Y¹⁵ is independently a hydrogen, a halo, a cyano, a hydroxyl, an optionally substituted C₁-C₆ alkyl, or an optionally substituted C₁-C₆ alkoxy; each Y²⁵ is independently a hydrogen or an optionally substituted C₁-C₆ alkyl, and each Y²⁰ is independently selected from the group consisting of:

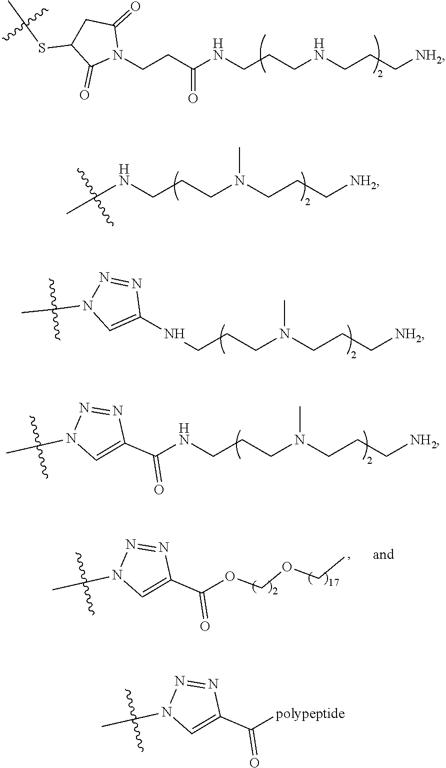

and polypeptide is a cationic polypeptide having about 5 to 30 amino acid residues.

In some embodiments, Z is

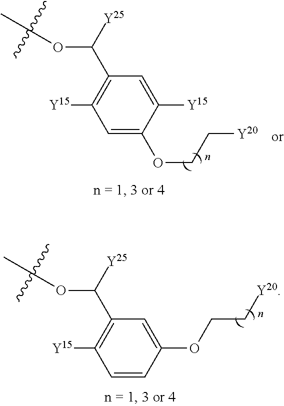

In some embodiments, Z is

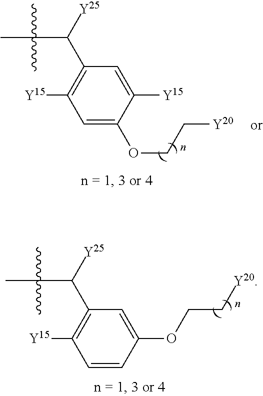

In some embodiments, each Y¹⁵ is independently a hydrogen. In some embodiments, each Y¹⁵ is independently a —NO₂. In some embodiments, each Y¹⁵ is independently a halo. In some embodiments, each Y¹⁵ is independently a cyano. In some embodiments, each Y¹⁵ is independently a hydroxyl. In some embodiments, each Y¹⁵ is independently an optionally substituted C₁-C₆ alkyl. In some embodiments, each Y¹⁵ is independently an optionally substituted C₁-C₆ alkoxy.

In some embodiments, each Y²⁵ is independently a hydrogen. In some embodiments, each Y²⁵ is independently an optionally substituted C₁-C₆ alkyl.

In some embodiments, each Y²⁰ is independently:

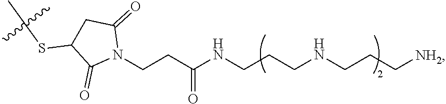

In some embodiments, each Y²⁰ is independently:

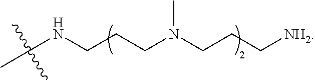

In some embodiments, each Y²⁰ is independently:

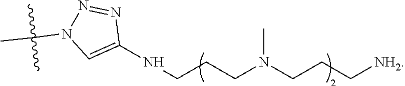

In some embodiments, each Y²⁰ is independently:

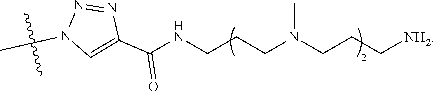

In some embodiments, each Y²⁰ is independently:

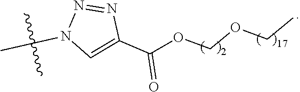

In some embodiments, each Y²⁰ is independently:

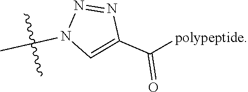

In some embodiments, Z is:

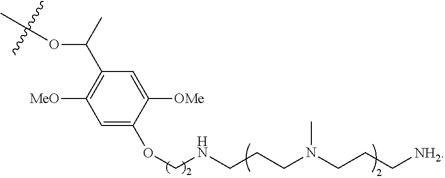

In some embodiments, Z is

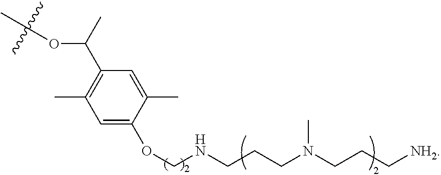

In some embodiments, Z is

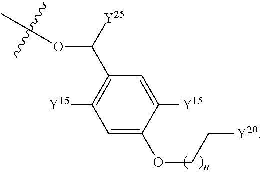

In some embodiments, Z is

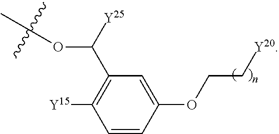

In some embodiments, Z is

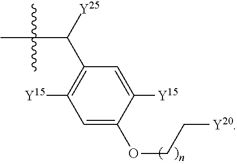

In some embodiments, Z is

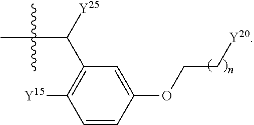

In some embodiments, n is 1, 2, 3, or 4. In some embodiments, n is 1, 3, or 4. In some embodiments, n is 1. In some embodiments, n is 2. In some embodiments, n is 3. In some embodiments, n is 4.

In some embodiments, each Y¹⁵ is independently a hydrogen. In some embodiments, each Y¹⁵ is independently a —NO₂. In some embodiments, each Y¹⁵ is independently a halo. In some embodiments, each Y¹⁵ is independently a cyano. In some embodiments, each Y¹⁵ is independently a hydroxyl. In some embodiments, each Y¹⁵ is independently an optionally substituted C₁-C₆ alkyl. In some embodiments, each Y¹⁵ is independently an optionally substituted C₁-C₆ alkoxy.

In some embodiments, each Y²⁵ is independently a hydrogen. In some embodiments, each Y²⁵ is independently an optionally substituted C₁-C₆ alkyl.

In some embodiments, each Y²⁰ is independently:

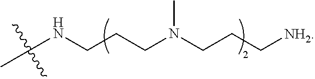

In some embodiments, each Y²⁰ is independently:

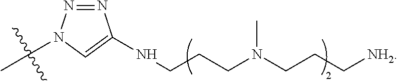

In some embodiments, each Y²⁰ is independently:

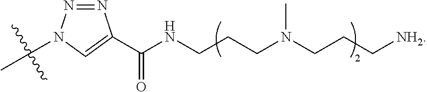

In some embodiments, each Y²⁰ is independently:

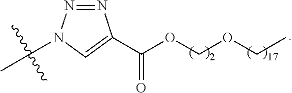

In some embodiments, each Y²⁰ is independently:

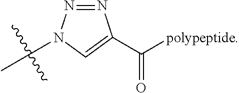

In some embodiments, Z is:

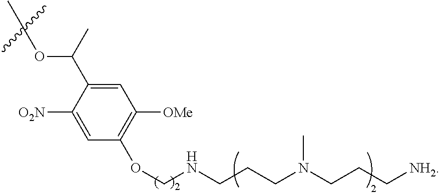

In some embodiments, Y⁴⁰ is a hydrogen. In some embodiments, Y⁴⁰ is an optionally substituted C₁-C₆ alkyl. In some embodiments, Y⁴⁰ is an optionally substituted C₂-C₁₀ alkenyl. In some embodiments, Y⁴⁰ is an optionally substituted C₂-C₁₀ alkynyl. In some embodiments, Y⁴⁰ is an optionally substituted C₆-C₁₀ aryl. In some embodiments, Y⁴⁰ is an optionally substituted 5-15 membered heteroaryl. In some embodiments, Y⁴⁰ is -L¹Y³⁵, wherein L¹ is as defined above. In some embodiments, Y³⁵ is a hydroxyl. In some embodiments, Y³⁵ is an optionally substituted C₁-C₆ alkoxy.

In some embodiments, L⁵ is a hydrogen. In some embodiments, L⁵ is an optionally substituted C₁-C₆ alkyl. In some embodiments, L⁵ is -L¹Y³⁵, wherein each of L¹ and Y³⁵ are as defined as above.

In some embodiments, Y³⁰ is a hydrogen. In some embodiments, Y³⁰ is an optionally substituted C₁-C₆ alkyl. In some embodiments, Y³⁰ is an optionally substituted C₂-C₁₀ alkenyl. In some embodiments, Y³⁰ is an optionally substituted C₂-C₁₀ alkynyl. In some embodiments, Y³⁰ is an optionally substituted C₆-C₁₀ aryl. In some embodiments, Y³⁰ is an optionally substituted 5-15 membered heteroaryl. In some embodiments, Y³⁰ is -L¹Y³⁵, wherein each of L¹ and Y³⁵ are as defined above

In some embodiments, L¹⁰ is a hydrogen. In some embodiments, L¹⁰ is an optionally substituted C₁-C₆ alkyl. In some embodiments, L¹⁰ is -L¹Y³⁵, wherein each of L¹ and Y³⁵ are as defined as above.

In some embodiments, R³ and R⁴ are H or Z. In some embodiments, R³ and R⁴ are H. In some embodiments, R³ and R⁴ are Z.

In some embodiments, R¹ and R² are H or Z. In some embodiments, R¹ and R² are H. In some embodiments, R¹ and R² are Z.

In some embodiments, X is O.

In some embodiments, X⁵ is O.

In some embodiments, L¹ is —PO₂— or —PO₃—PO₂—. In some embodiments, L¹ is —PO₃—PO₂—.

In some embodiments, each R⁵, R⁶, R⁷, R⁸, R⁹, R¹⁰, and R¹¹ is independently selected from —N₃, a hydroxyl, an optionally substituted C₂-C₁₀ alkynyl, an optionally substituted C₁-C₁₀ alkoxy, —SR³⁰, an optionally substituted 5-15 membered heteroaryl, or Z.

In some embodiments, at least one of R⁶ and R⁷ and at least one of R⁸ and R⁹ is independently selected from —N₃, a hydroxyl, an optionally substituted C₂-C₁₀ alkynyl, an optionally substituted C₁-C₁₀ alkoxy, —SR³⁰, an optionally substituted 5-15 membered heteroaryl, or Z.

In some embodiments, R⁶ and R⁸ is independently selected from —N₃, a hydroxyl, an optionally substituted C₂-C₁₀ alkynyl, an optionally substituted C₁-C₁₀ alkoxy, —SR³⁰, an optionally substituted 5-15 membered heteroaryl, or Z.

In some embodiments, each R⁵, R⁶, R⁷, R⁸, R⁹, R¹⁰, and R¹¹ is independently selected from —N₃, a hydroxyl, an optionally substituted C₂-C₁₀ alkoxy, or an optionally substituted 5-15 membered heteroaryl.

In some embodiments, at least one of R⁶ and R⁷ and at least one of R⁸ and R⁹ is independently selected from —N₃, a hydroxyl, an optionally substituted C₂-C₁₀ alkoxy, or an optionally substituted 5-15 membered heteroaryl.

In some embodiments, R⁶ and R⁸ is independently selected from —N₃, a hydroxyl, an optionally substituted C₂-C₁₀ alkoxy, or an optionally substituted 5-15 membered heteroaryl.

In some embodiments, the optionally substituted 5-15 membered heteroaryl is an optionally substituted 6-10 membered heteroaryl. In some embodiments, the 6-10 membered heteroaryl is optionally substituted with one, two, three, four, or five R¹⁵ groups, as defined below. In some embodiments, the 6-10 membered heteroaryl is optionally substituted with an aminocarbonyl group.

In some embodiments, the optionally substituted 5-15 membered heteroaryl is an optionally substituted 6 membered heteroaryl. In some embodiments, the 6 membered heteroaryl is optionally substituted with one, two, three, four, or five R¹⁵ groups, as defined below. In some embodiments, the 6 membered heteroaryl is optionally substituted with an aminocarbonyl group.

In some embodiments, the optionally substituted 5-15 membered heteroaryl is an optionally substituted pyridyl. In some embodiments, the pyridyl group is optionally substituted with one, two, three, four, or five R¹⁵ groups, as defined below. In some embodiments, the pyridyl is optionally substituted with an aminocarbonyl group.

In some embodiments, the optionally substituted 5-15 membered heteroaryl is:

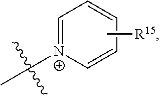

wherein R¹⁵ is —C(O)NR⁶⁰R⁶¹, —OC(O)NR⁶⁰R⁶¹, —C(S)NR⁶⁰R⁶¹, or —OC(S)NR⁶⁰R⁶¹.

In some embodiments, R¹⁵ is —C(O)NR⁶⁰R⁶¹. In some embodiments, R¹⁵ is —OC(O)NR⁶⁰R⁶¹. In some embodiments, R¹⁵ is —C(S)NR⁶⁰R⁶¹. In some embodiments, R¹⁵ is —OC(S)NR⁶⁰R⁶¹.

In some embodiments, each R⁶⁰ and R⁶¹ is a hydrogen or an optionally substituted C₁-C₆ alkyl. In some embodiments, R⁶⁰ is a hydrogen. In some embodiments, R⁶⁰ is an optionally substituted C₁-C₆ alkyl. In some embodiments, R⁶¹ is a hydrogen. In some embodiments, R⁶¹ is an optionally substituted C₁-C₆ alkyl. In some embodiments, R⁶⁰ and R⁶¹ are hydrogen.

In some embodiments, each R⁵, R⁶, R⁷, R⁸, R⁹, R¹⁰, and R¹¹ independently is selected from the group consisting of:

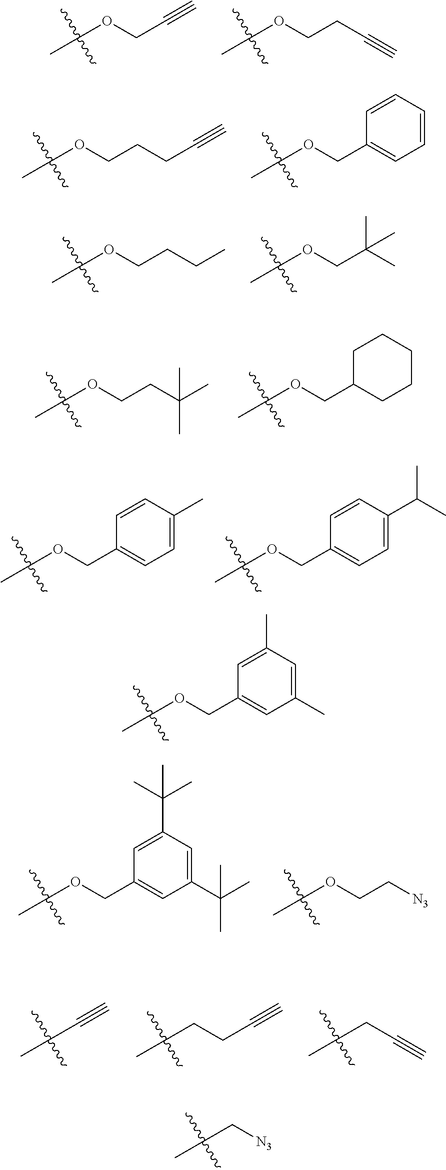

In some embodiments, R⁵ is:

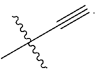 [SMILES: *C#C]

In some embodiments, R⁵ is:

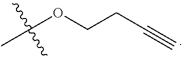 [SMILES: *OCCC#C]

In some embodiments, at least one of R⁶ and R⁷ is a hydroxyl.

In some embodiments, at least one of R⁶ and R⁷ is:

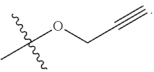 [SMILES: *OCC#C]

In some embodiments, at least one of R⁶ and R⁷ is:

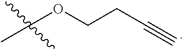 [SMILES: *OCCC#C]

In some embodiments, at least one of R⁶ and R⁷ is:

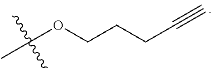 [SMILES: *OCCCC#C]

In some embodiments, at least one of R⁶ and R⁷ is:

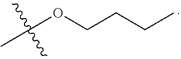 [SMILES: *OCCCC]

In some embodiments, at least one of R⁶ and R⁷ is:

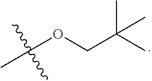 [SMILES: *OCC(C)(C)C]

In some embodiments, at least one of R⁶ and R⁷ is:

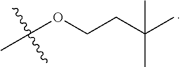 [SMILES: *OCCC(C)(C)C]

In some embodiments, at least one of R⁶ and R is:

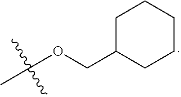 [SMILES: *OCC1CCCCC1]

In some embodiments, at least one of R⁶ and R⁷ is:

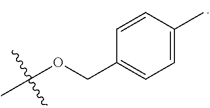 [SMILES: *OCc1ccc(C)cc1]

In some embodiments, at least one of R⁶ and R⁷ is:

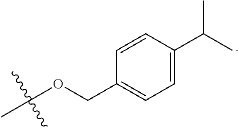 [SMILES: *OCc1ccc(C(C)C)cc1]

In some embodiments, at least one of R⁶ and R⁷ is:

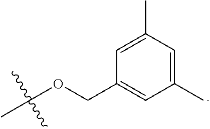 [SMILES: *OCc1cc(C)cc(C)c1]

In some embodiments, at least one of R⁶ and R⁷ is:

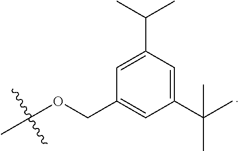 [SMILES: *OCc1cc(C(C)C)cc(C(C)(C)C)c1]

In some embodiments, at least one of R⁶ and R⁷ is —N₃.

In some embodiments, at least one of R⁸ and R⁹ is hydroxyl.

In some embodiments, at least one of R⁸ and R⁹ is:

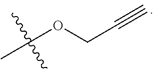 [SMILES: *OCC#C]

In some embodiments, at least one of R⁸ and R⁹ is:

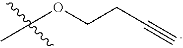 [SMILES: *OCCC#C]

In some embodiments, at least one of R⁸ and R⁹ is:

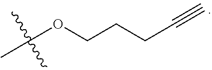 [SMILES: *OCCCC#C]

In some embodiments, at least one of R⁸ and R⁹ is:

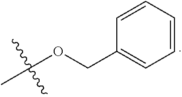 [SMILES: *OCc1ccccc1]

In some embodiments, at least one of R⁸ and R⁹ is:

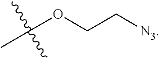 [SMILES: *OCCN=[N+]=[N-]]

In some embodiments, at least one of R⁸ and R⁹ is:

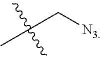 [SMILES: *CN=[N+]=[N-]]

In some embodiments, at least one of R⁸ and R⁹ is —N₃.

In some embodiments, at least one of R⁶, R⁸, and R¹⁰ is Z.

In some embodiments, at least one of R⁶ and R⁸ is Z.

In some embodiments, R⁶ and R⁸ are hydroxyl.

In some embodiments, R¹⁰ is:

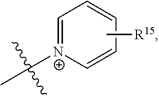

wherein R¹⁵ is as defined above.

In some embodiments, R¹⁰ is:

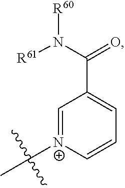

wherein R⁶⁰ and R⁶¹ are as defined above.

In some embodiments, R¹¹ is:

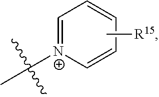

wherein R¹⁵ is as defined above.

In some embodiments, R¹¹ is:

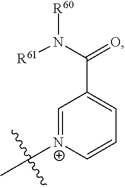

wherein R⁶⁰ and R⁶¹ are as defined above.

In another aspect, provided herein is a compound selected from Tables 1-3 below.

TABLE 1

| | |
|---|---|
| 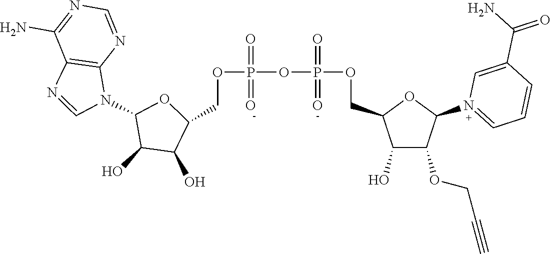 | NAD⁺ 1 |
| 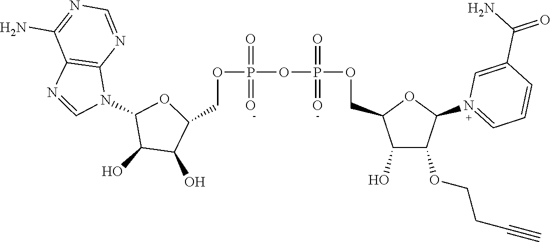 | NAD⁺ 2 |
| 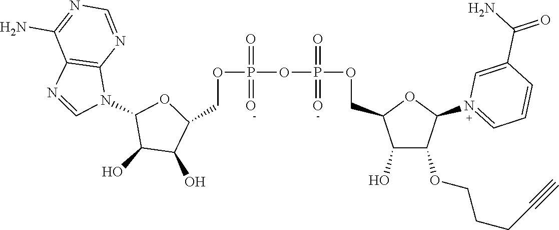 | NAD⁺ 3 |
| 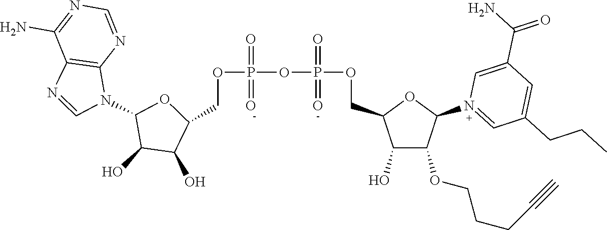 | NAD⁺ 4 |
| 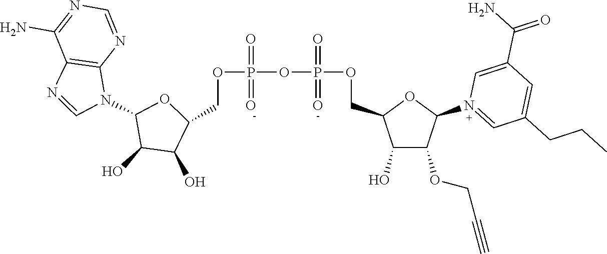 | NAD⁺ 5 |
| 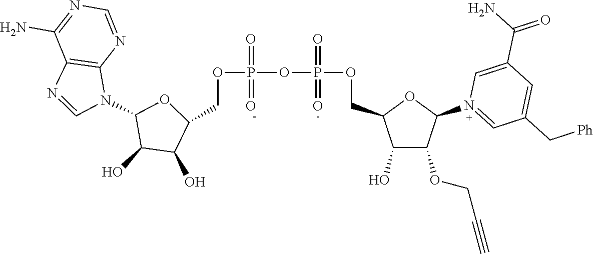 | NAD⁺ 6 |
| 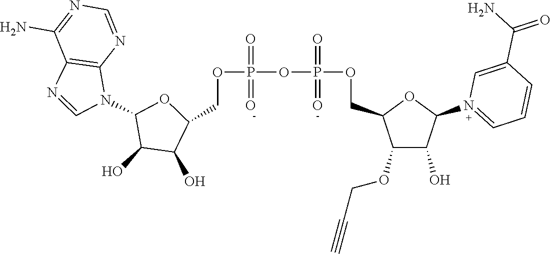 | NAD⁺ 7 |
| 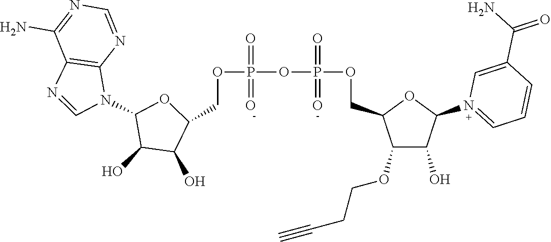 | NAD⁺ 8 |
| 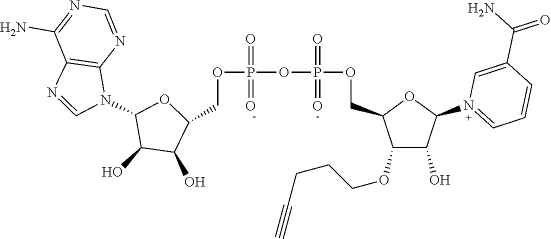 | NAD⁺ 9 |
| 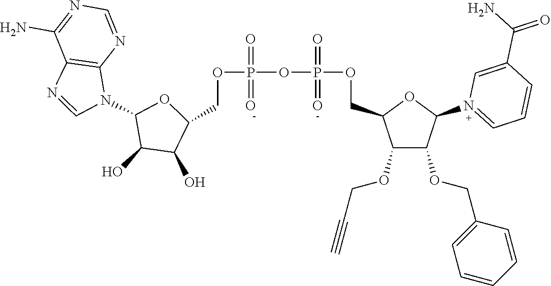 | NAD⁺ 10 |
| 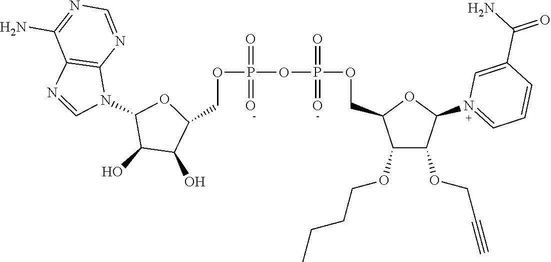 | NAD⁺ 11 |
| 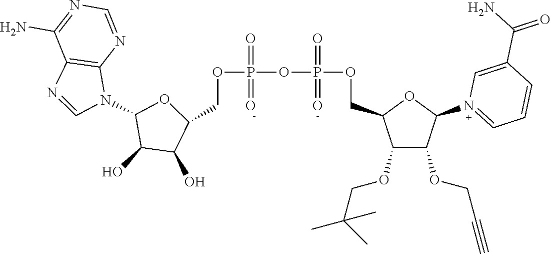 | NAD⁺ 12 |

TABLE 2

| | |
|---|---|
| 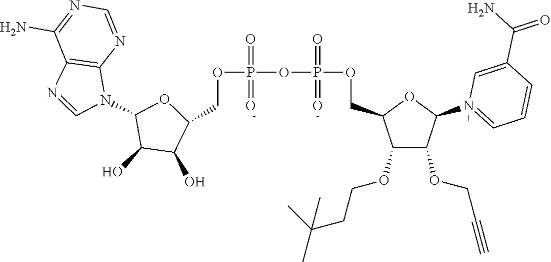 | NAD⁺ 13 |
| 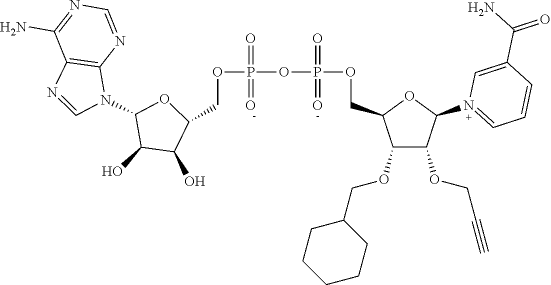 | NAD⁺ 14 |
| 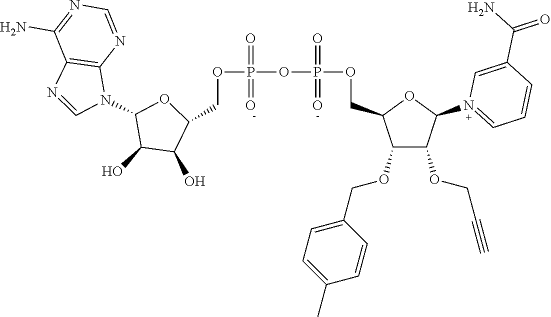 | NAD⁺ 15 |
| 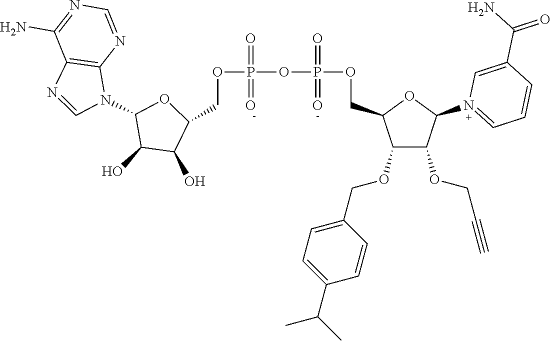 | NAD⁺ 16 |
| 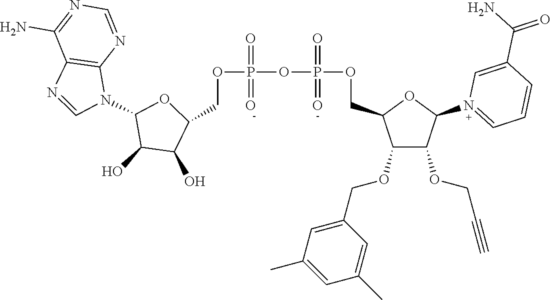 | NAD⁺ 17 |
| 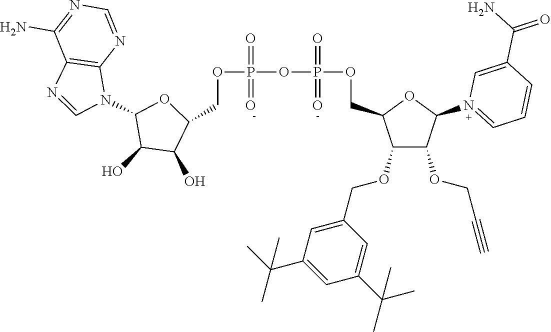 | NAD⁺ 18 |
| 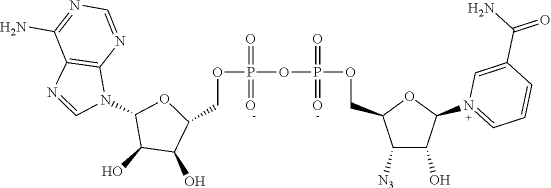 | NAD⁺ 19 |
| 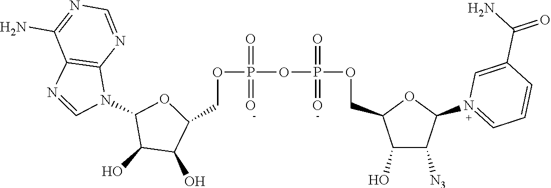 | NAD⁺ 20 |
| 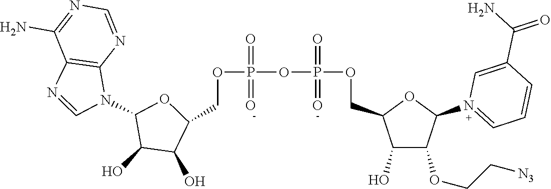 | NAD⁺ 21 |
| 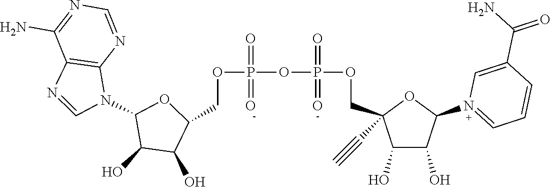 | NAD⁺ 22 |
| 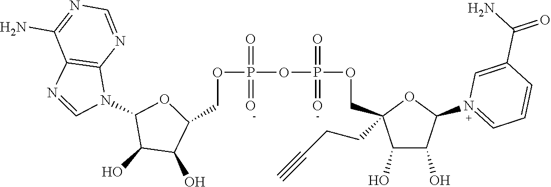 | NAD⁺ 23 |
| 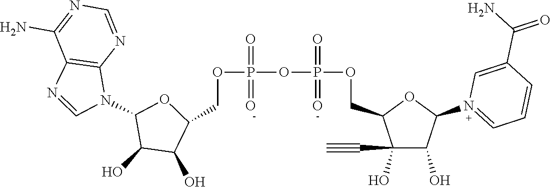 | NAD⁺ 24 |

TABLE 3

| | |
|---|---|
| 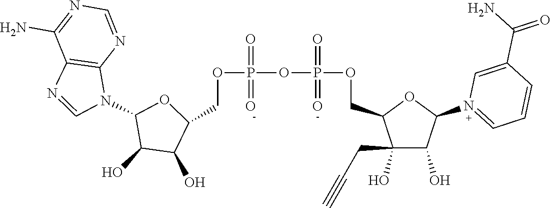 | NAD⁺ 25 |
| 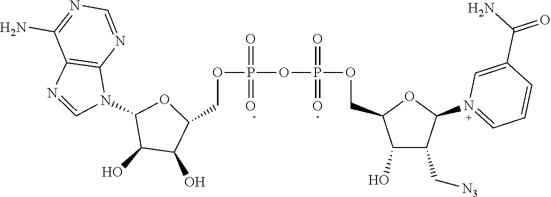 | NAD⁺ 26 |
| 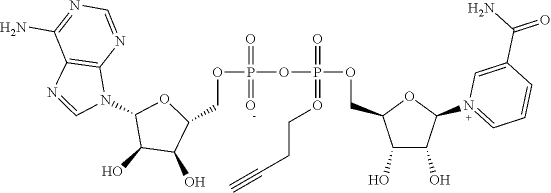 | NAD⁺ 27 |
| 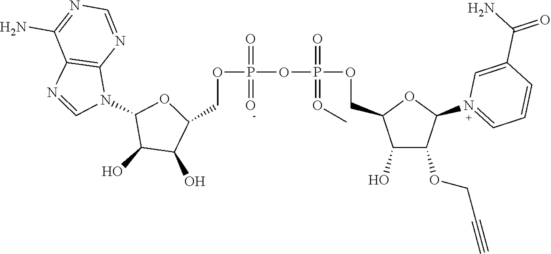 | NAD⁺ 28 |
| 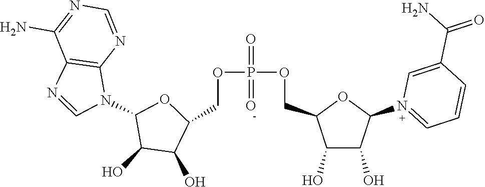 | NAD⁺ 29 |
| 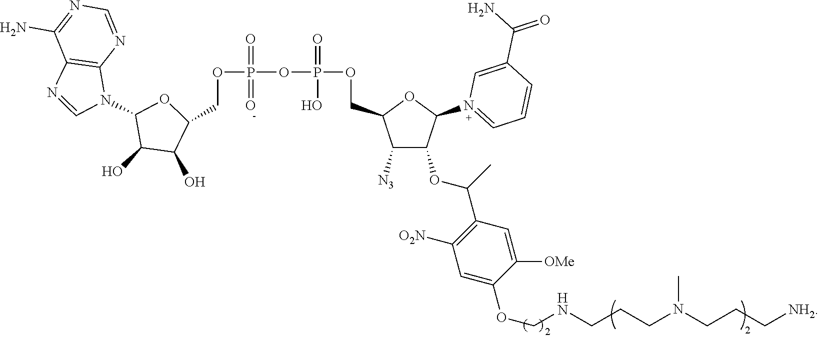 | NAD⁺ 30 |

In another aspect, provided herein is a compound selected from Tables 1-4.

TABLE 4

| | |
|---|---|
| 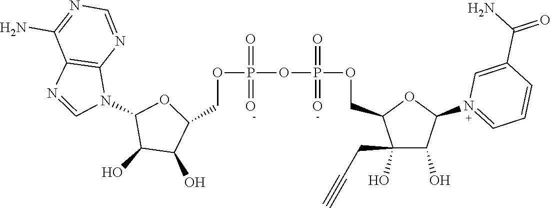 | NAD⁺ 25 |
| 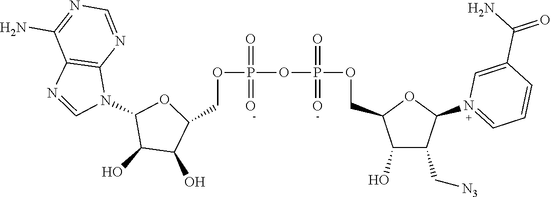 | NAD⁺ 26 |
| 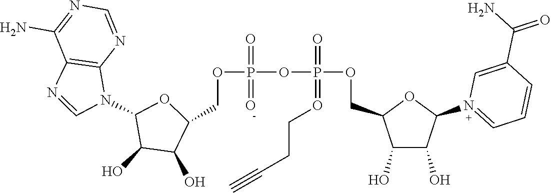 | NAD⁺ 27 |
| 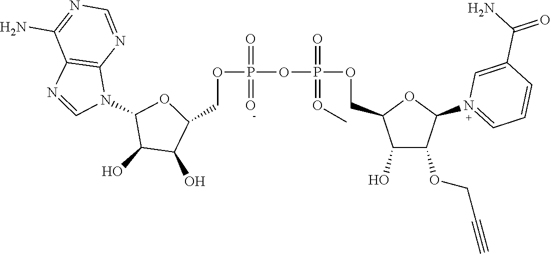 | NAD⁺ 28 |
| 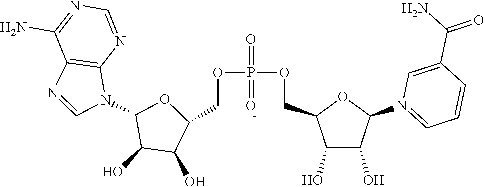 | NAD⁺ 29 |
| 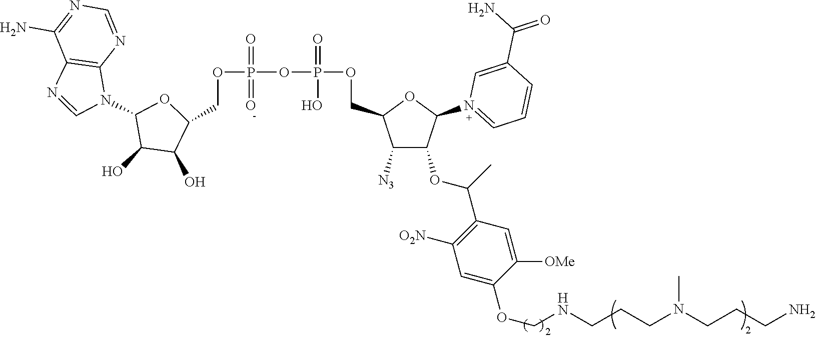 | NAD⁺ 30 |
| 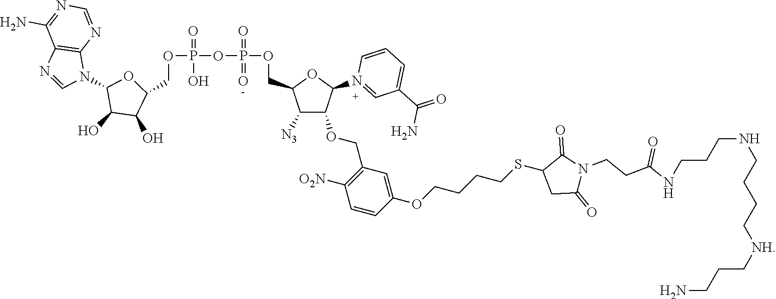 | NAD⁺ 31 |

The compounds provided herein include individual, separated enantiomers and diastereomers that are stereochemically pure or enriched, tautomers, and pharmaceutically acceptable salts, and/or a solvate thereof, wherever applicable. As used herein, the term stereochemically pure denotes a compound which has 80% or greater by weight of the indicated stereoisomer and 20% or less by weight of other stereoisomers. In a further aspect, the compounds as described herein have 90% or greater by weight of the denoted stereoisomer and 10% or less by weight of other stereoisomers. In a yet further embodiment, the compounds of this disclosure have 95% or greater by weight of the denoted stereoisomer and 5% or less by weight of other stereoisomers. In a still further embodiment, the compounds have 97% or greater by weight of the denoted stereoisomer and 3% or less by weight of other stereoisomers. Any one or more of the compounds can be provided as compositions, e.g., of pharmaceutically acceptable salt, and/or a solvate thereof.

### Synthesis

The following general synthetic scheme is used to prepare the compounds provided herein. For example, compounds of formula I are synthesized as shown in the reaction scheme below:

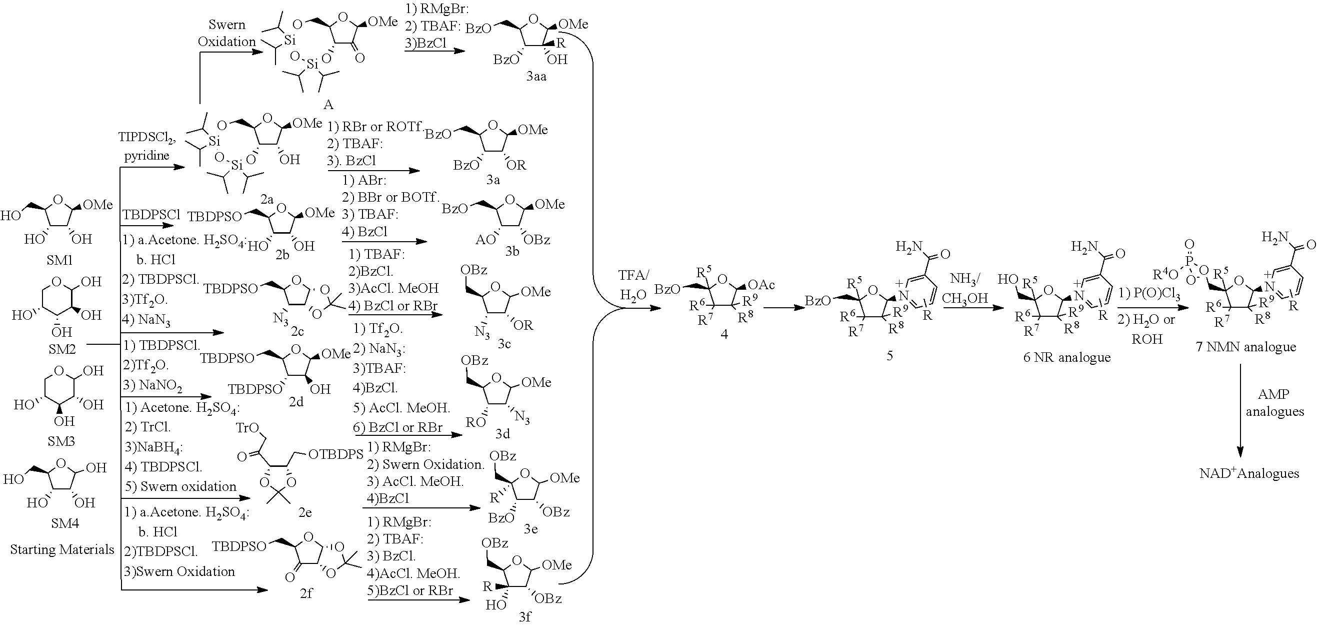

These and other compounds provided herein are synthesized following art recognized methods with the appropriate substitution of commercially available reagents as needed. For example, and without limitation, methods for synthesizing certain other compounds are described in J. Am. Chem. Soc. 2015, 137, 3558-3564; Angew. Chem. Int. Ed. 2014, 53, 8159-8162; J. Am. Chem. Soc. 2014, 136, 5201-5204; Nucleosides, Nucleotides and Nucleic Acids, 2013, 32, 646-659; Bioorganic & Medicinal Chemistry Letters, 2002, 12, 1135-1137; and Biochemistry, 2009, 48, 2878-2890, each of which are incorporated herein by reference, which methods can be adapted by the skilled artisan upon reading this disclosure and/or based on synthetic methods well known in the art, to prepare the compounds provided herein. Protection deprotection methods and protecting groups useful for such purposes are well known in the art, for example in Greene's Protective Groups in Organic Synthesis, 4^{th} Edition, Wiley, 2006, or a later edition of the book.

The compounds and the intermediates are separated from the reaction mixture, when desired, following art known methods such as crystallization, chromatography, distillation, and the like. The compounds and the intermediates are characterized by art known methods such as thin layer chromatography, nuclear magnetic resonance spectroscopy, high performance liquid chromatography, and the like. As described in detail herein, a racemic or diastereomeric mixture of the compound can be separated or enriched to the enantiomers and diastereomers and tested and used diagnostically or therapeutically as described herein.

Methods of testing and using the compounds provided herein are performed following art recognized in vitro (cell free), ex vivo or in vivo methods. For example, and without limitation, certain methods for testing and using other compounds are described in Carter-O'Connell, I., Jin, H., Morgan, R. K., David, L. L., and Cohen, M. S. (2014) Engineering the substrate specificity of ADP-ribosyltransferases for identifying direct protein targets. J. Am. Chem. Soc. 136, 5201-5204; Gibson, B. A., Zhang, Y., Jiang, H., Hussey, K. M., Shrimp, J. H., Lin, H., Schwede, F., Yu, Y., and Kraus, W. L. (2016) Chemical genetic discovery of PARP targets reveals a role for PARP-1 in transcription elongation. Science 353, 45-50; Wallrodt, S., Buntz, A., Wang, Y., Zumbusch, A., and Marx, A. (2016) Bioorthogonally Functionalized NAD(+) Analogues for In-Cell Visualization of Poly(ADP-Ribose) Formation. Angew. Chem. Int. Ed. Engl. 55, 7660-7664; and Buntz, A., Wallrodt, S., Gwosch, E., Schmalz, M., Beneke, S., Ferrando-May, E., Marx, A., and Zumbusch, A. (2016) Real-Time Cellular Imaging of Protein Poly(ADP-ribos)ylation. Angew. Chem. Int. Ed. Engl. 55, 11256-11260, each of which is incorporated herein by reference in its entirety, which methods can be adapted by the skilled artisan upon reading this disclosure and/or based on methods well known in the art, to test and use the compounds provided herein.

### Compositions

Compositions, including pharmaceutical compositions comprising the compounds described herein can be manufactured by means of conventional mixing, dissolving, granulating, dragee-making levigating, emulsifying, encapsulating, entrapping, or lyophilization processes. The compositions can be formulated in conventional manner using one or more physiologically acceptable carriers, diluents, excipients, or auxiliaries which facilitate processing of the compounds provided herein into preparations which can be used pharmaceutically.

The compounds of the technology can be administered by admixing in an in vitro system, parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration.

In one embodiment, this disclosure relates to a composition comprising a compound as described herein and a carrier.

In another embodiment, this disclosure relates to a pharmaceutical composition comprising a compound as described herein and a pharmaceutically acceptable carrier.

In another embodiment, this disclosure relates to a pharmaceutical composition comprising a therapeutically effective amount of a compound as described herein and a pharmaceutically acceptable carrier.

The pharmaceutical compositions for the administration of the compounds can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition the compound provided herein is included in an amount sufficient to produce the desired therapeutic effect. For example, pharmaceutical compositions of this disclsoure may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the compounds can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the compounds provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

### Use of Compounds for Preparing Medicaments

The compounds and compositions of the present invention are also useful in the preparation of medicaments. The methods and techniques for preparing medicaments of a composition are known in the art. For the purpose of illustration only, pharmaceutical formulations and routes of delivery are detailed herein.

Thus, one of skill in the art would readily appreciate that any one or more of the compositions described above, including the many specific embodiments, can be used by applying standard pharmaceutical manufacturing procedures to prepare medicaments to treat the many disorders described herein. Such medicaments can be delivered to the subject by using delivery methods known in the pharmaceutical arts.

### Methods of Use

The compositions and compounds as disclosed herein are useful in assay and detection methods in vitro in a cell-free system or in vivo in a cell or subject, wherein the cell-free system, in vivo in a cell or subject, also comprise a PARP enzyme and a possible substrate protein for the PARP enzyme.

In one aspect, provided herein are methods of monitoring and/or tracking ADP-ribosylation in the above-noted cell-free system, a cell, a live cell, a tissue or a subject. In one aspect, the subject is, or the cell is isolated from, or cultured from, an animal, e.g., a mammal such as a human, a murine, a canine, a feline, an equine, an ovine, or a bovine. In some embodiments, the subject is a human. The cells can be from commercially available or laboratory generated cell lines or isolated from a subject and used to monitor therapy, e.g., a tissue biopsy. Thus, the assays can be performed at various time points using samples isolated from the same subject.

The compositions and compounds as disclosed herein are useful in methods of identifying and profiling substrates of ADP-ribosyltransferases in a cell-free system, a cell, a tissue or a subject. The compositions and compounds as disclosed herein also are useful in methods of modulating enzymatic activities of ADP-ribosyltransferases in a cell-free system, a cell, a tissue or a subject.

The compositions and compounds as disclosed herein are useful in methods of modulating the levels, extents, and patterns of ADP-ribosylation in a subject in need thereof. In one aspect, the subject is, or the cell is isolated from, or cultured from, an animal, e.g., a human, a mammal such as a murine, a canine, a feline, an equine, an ovine, or a bovine.

The compositions and compounds as disclosed herein are useful in methods of modulating the metabolism of cellular NAD⁺ and its associated metabolic and signaling pathways in a cell-free system, a cell, a tissue or a subject. In one aspect, the subject is, or the cell is isolated from, or cultured from, an animal, e.g., a human, a mammal such as a murine, a canine, a feline, an equine, an ovine, or a bovine.

The compositions and compounds as disclosed herein are useful in methods of modulating post-translational modifications related to NAD⁺ cofactor in a cell-free system, a cell, a tissue or a subject. In one aspect, the subject is, or the cell is isolated from, or cultured from, an animal, e.g., a mammal such as a human, a murine, a canine, a feline, an equine, an ovine, or a bovine. In some embodiments, the post-translational modification is protein acetylation. In some embodiments, the post-translational modification is protein succinylation. Other examples of post-translational modifications are well known in the art.

The compositions and compounds as disclosed herein are useful in methods of purifying a PARP substrate protein from a cell, a tissue or a subject. In one aspect, the subject is, or the cell is isolated from, or cultured from, an animal, e.g., a mammal such as a human, a murine, a canine, a feline, an equine, an ovine, or a bovine. The methods include contacting a sample comprising the protein, PARP and a compound of disclosed herein under conditions that favor PARP enzyme activity and with an affinity label to produce ADP-ribosylated protein, where the ribose is labeled, and affinity purifying the ADP-ribosylated protein. In one aspect, the click chemistry is used to label the protein.

The compositions and compounds as disclosed herein are useful in methods of identifying a protein as a substrate for PARP in a cell, a tissue or a subject. In one aspect, the subject is, or the cell or tissue is isolated from, or cultured from, an animal, e.g., a mammal such as a human, a murine, a canine, a feline, an equine, an ovine, or a bovine. In some embodiments, the subject is a human.

The disclosed methods can be performed in a cell free system with a cellular extract containing a PARP, or in a cell or tissue culture, or in a subject. When performed in vitro or in a cell free system, the methods include contacting a sample including the PARP, and a compound disclosed herein under conditions for PARP to act on a substrate (e.g. a histone or non-histone protein, and with an affinity or detectable label to a labeled product of the PARP reaction. In one aspect) the affinity label is used to purify the ADP-ribosylated protein; and the method optionally comprises further characterizing the ADP-ribosylated protein. Methods to characterize the protein are known in the art, non-limiting examples of such include e.g., mass spectrometry, liquid or gas chromatography/mass spectrometry, nuclear magnetic resonance imaging. Alternatively, the method uses a detectable label which is then imaged based on the label used. Suitable labels are known in the art and described herein, e.g., the label comprises an alkyne. In some embodiments the label comprises an azide. In some embodiments the label is a fluorescent label, e.g., a fluorophore. Non-limiting examples of detectable labels are describe herein. When performed in vivo, an effective amount of the compound and label can be administered to the subject. When practiced in a non-human animal, e.g., an appropriate mouse model, the method can be used to screen for novel combination therapies, formulations or treatment regimens, prior to administration to a human patient.

The compositions and compounds as disclosed herein also are useful in methods of labeling a PARP substrate protein in a cell-free system, a cell, a tissue or a subject. This disclosure also provides the labeled products produced therefrom. In one aspect, the subject is, or the cell or tissue is isolated from, or cultured from, an animal, e.g., a mammal such as a human, a murine, a canine, a feline, an equine, an ovine, or a bovine. When performed in vitro, the methods include contacting a sample comprising a PARP with a compound of this disclosure under conditions that allow the activity of the PARP enzyme, and a detectable label. Suitable labels are known in the art and described herein, e.g., the label comprises an alkyne. In some embodiments the label comprises an azide. In some embodiments the label is a fluorescent label, e.g., a fluorophore.

In some embodiments the compound of the disclosure contacted with PARP comprises a coupling moiety configured to couple with a complementary coupling moiety of the label. In some embodiments the compound of the disclosure contacted with PARP comprises a pi-system capable of reacting with a pi-system of the label. In some embodiments, the reaction of the two pi-systems is a cycloaddition reaction. In some embodiments the compound of the disclosure contacted with PARP comprises an alkyne and the label comprises an azide. In some embodiments the compound of the disclosure contacted with PARP comprises an azide and the label comprises an alkyne. In some embodiments, the alkyne is a terminal alkyne.

When practiced in vivo in a patient such as an animal or human, the compounds and labels are administered in an effective amount by a suitable route of administration. When practiced in a non-human animal, e.g., an appropriate mouse model, the method can be used to screen for novel combination therapies, formulations or treatment regimens, prior to administration to a human patient.

### Kits

The compounds and compositions, as described herein, can be provided in kits. The kits can further contain instructions for use.

The following examples are included to demonstrate some embodiments of the disclosure. However, those of skill in the art should, in light of the present disclosure, appreciate that many changes can be made in the specific embodiments which are disclosed and still obtain a like or similar result without departing from the spirit and scope of the disclosure.

### EXAMPLES

### Example 1. Synthesis of NAD⁺1

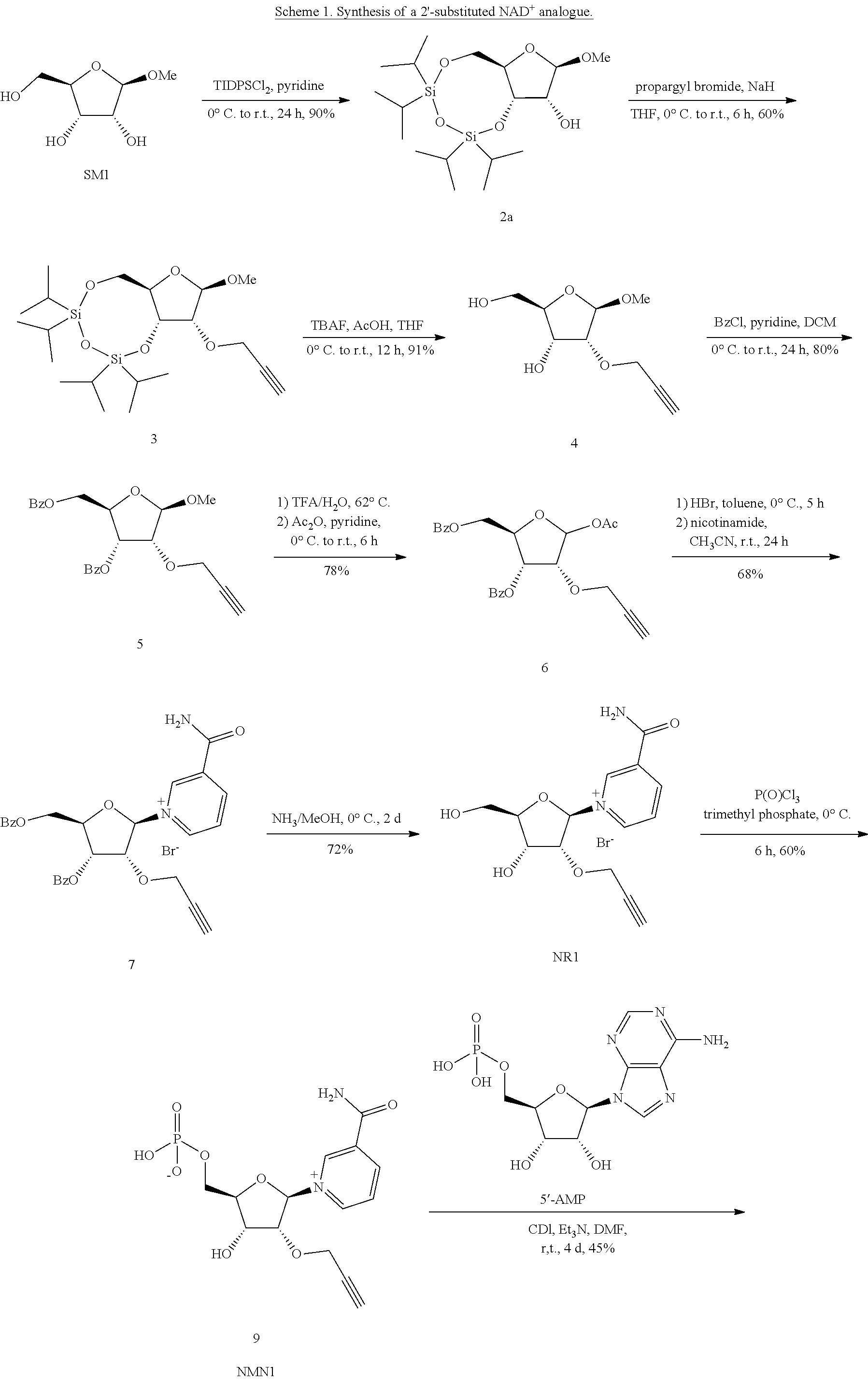

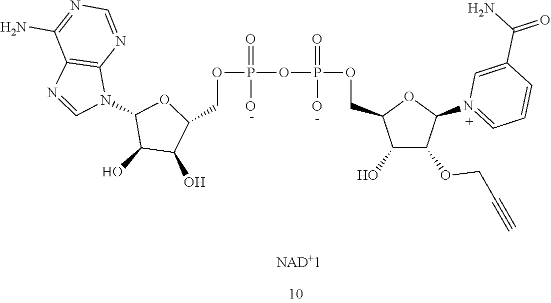

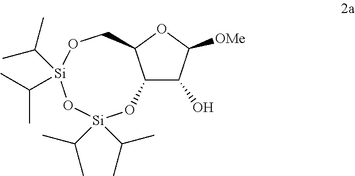 [SMILES: CO[C@@H]1O[C@@H]2CO[Si](C(C)C)(C(C)C)O[Si](C(C)C)(C(C)C)O[C@H]2[C@H]1O]

General procedure for the synthesis of (6aR,8R,9R,9aR)-2,2,4,4-tetraisopropyl-8-methoxy-7-ol-tetrahydro-6H-furo[3,2-f][1,3,5,2,4]trioxadisilocine (2a): To a stirred solution of Methyl β-D-ribofuranoside (SM1) (3.1 g, 19.0 mmol) in pyridine (24 mL) was added the TIDPSCl₂ (9.0 g, 28.5 mmol) at 0° C. The reaction mixture was allowed to warm to room temperature. After stirring at this temperature for 24 hours, the reaction mixture was diluted with EtOAc (100 mL), and the organic phase was washed successively with ice-water (50 mL), aq 1M HCl (2×50 mL), H₂O (2×50 mL), dried over anhydrous Na₂SO₄, filtered and concentrated to give a residue. The residue was purified by a flash column chromatography on silica gel to afford the compound (2a) (7.0 g, 90%) as a colorless oil.

(6aR,8R,9R,9aR)-2,2,4,4-tetraisopropyl-8-methoxy-7-ol-tetrahydro-6H-furo[3,2-f][1,3,5,2,4]trioxadisilocine (2a). A colorless oil, 90% yield; ¹H NMR (400 MHz, CDCl₃): δ 1.06-1.10 (m, 28H, 4CH+8CH₃), 3.31 (s, 3H, OCH₃), 3.75 (dd, 1H, J=10.8, 8.8 Hz, CH₂), 3.99-4.07 (m, 3H, CH₂+2CH), 4.50 (t, 1H, J=5.2 Hz, CH), 4.82 (s, 1H, CH); ¹³C NMR (100 MHz, CDCl₃): δ 12.5, 12.8, 13.25, 13.27, 16.94, 16.97, 17.0, 17.2, 17.36, 17.38, 17.4, 17.5, 54.9, 66.2, 75.0, 75.7, 82.7, 107.2.

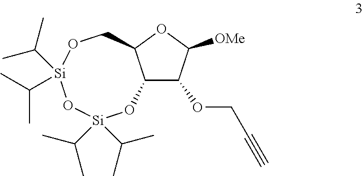 [SMILES: C#CCO[C@H]1[C@H](OC)O[C@@H]2CO[Si](C(C)C)(C(C)C)O[Si](C(C)C)(C(C)C)O[C@@H]12]

General procedure for the synthesis of (6aR,8R,9R,9aR)-2,2,4,4-tetraisopropyl-8-methoxy-9-(prop-2-yn-1-yloxy)tetrahy-dro-6H-furo[3,2-f][1,3,5,2,4]trioxadisilocine (3): To a stirred solution of compound (2a) (1.5 g, 3.7 mmol) in anhydrous THF (25 mL) was added NaH (180 mg, 4.5 mmol, 1.2 eq, 60% dispersion in mineral oil) at 0° C. followed by the addition of propargyl bromide (660 mg, 5.6 mmol, 1.5 eq) at the same temperature. Then reaction mixture was allowed to warm to room temperature. After stirring at this temperature for 6 hours, the reaction mixture was quenched with saturated aqueous NH₄Cl (20 mL) and extracted with EtOAc (3×50 mL). The combined organic layers were washed water (3×50 mL), dried over anhydrous Na₂SO₄, filtered and concentrated and purified by a flash column chromatography on silica gel to afford the compound (3) (987 mg, 60%) as a colorless oil.

(6aR,8R,9R,9aR)-2,2,4,4-tetraisopropyl-8-methoxy-9-(prop-2-yn-1-yloxy)tetrahy-dro-6H-furo[3,2-f][1,3,5,2,4]trioxadisilocine (3). A colorless oil, 60% yield; ¹H NMR (400 MHz, CDCl₃): δ 1.02-1.08 (m, 28H, 4CH+8CH₃), 2.43 (t, 1H, J=2.4 Hz, CH), 3.32 (s, 3H, OCH₃), 3.84-3.88 (m, 1H, CH₂), 3.96-4.02 (m, 3H, CH₂+2CH), 4.42-4.51 (m, 3H, CH₂+CH), 4.77 (s, 1H, CH); ¹³C NMR (100 MHz, CDCl₃): δ 12.6, 12.7, 13.1, 13.3, 16.97, 17.01, 17.09, 17.17, 17.29, 17.30, 17.4, 54.7, 58.2, 63.6, 73.9, 74.8, 79.7, 80.6, 80.9, 105.9.

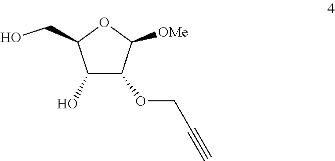

General procedure for the synthesis of (2R,3R,4R,5R)-2-(hydroxymethyl)-5-methoxy-4-(prop-2-yn-1-yloxy)tetrahydrofuran-3-ol (4): To a 0° C. solution of compound (3) (934 mg, 2.1 mmol) in anhydrous THF (25 mL) was added AcOH (180 μL, 3.2 mmol, 1.5 eq) followed by the addition of TBAF (3.2 mL, 3.2 mmol, 1.0 M in THF, 1.5 eq). Then reaction mixture was allowed to warm to room temperature. After stirring at this temperature for 14 hours, the reaction mixture was concentrated under reduced pressure to give a residue. The residue was purified by a flash column chromatography on silica gel to afford the compound (4) (386 mg, 91%) as a colorless oil.

(2R,3R,4R,5R)-2-(hydroxymethyl)-5-methoxy-4-(prop-2-yn-1-yloxy)tetrahydro-furan-3-ol (4). A colorless oil, 91% yield; ¹H NMR (400 MHz, CDCl₃): δ 2.52 (t, 1H, J=2.4 Hz, CH), 3.43 (s, 3H, OCH₃), 3.64 (dd, 1H, J=12.4, 3.6 Hz, CH₂), 3.80 (dd, 1H, J=12.4, 2.4 Hz, CH), 3.99 (dd, 1H, J=5.6, 1.2 Hz, CH), 4.05-4.08 (m, 1H, CH), 4.28-4.39 (m, 3H, CH₂+CH), 4.98 (s, 1H, CH); ¹³C NMR (100 MHz, CDCl₃): δ 55.9, 58.5, 63.0, 70.9, 75.7, 78.7, 82.7, 85.6, 106.5.

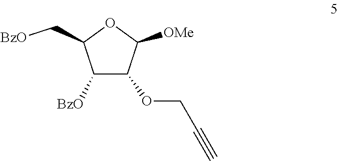

General procedure for the synthesis of ((2R,3R, 4R, 5R)-3-(benzoyloxy)-5-methoxy-4-(prop-2-yn-1-yloxy)tetrahydrofuran-2-yl)methyl benzoate (5): To a solution of compound (4) (320 mg, 1.7 mmol) in a mixture of anhydrous DCM (10 mL) and anhydrous pyridine (10 mL) was added BzCl (588 μL, 5.1 mmol, 3 eq) at 0° C. Then the reaction mixture was allowed to warm to room temperature. After stirring for 24 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the compound (5) (558 mg, 80%) as a colorless oil.

((2R,3R, 4R, 5R)-3-(benzoyloxy)-5-methoxy-4-(prop-2-yn-1-yloxy)tetrahydrofuran-2-yl)methyl benzoate (5). A colorless oil, 91% yield; ¹H NMR (400 MHz, CDCl₃): δ 2.39 (t, 1H, J=2.4 Hz, CH), 3.38 (s, 3H, OCH₃), 4.23 (dd, 1H, J=16.0, 2.4 Hz, CH₂), 4.29 (dd, 1H, J=16.0, 2.4 Hz, CH₂), 4.40 (dd, 1H, J=4.8, 1.2 Hz, CH), 4.43-4.49 (m, 1H, CH₂), 4.59-4.66 (m, 2H, CH₂+CH), 5.08 (d, 1H, J=0.4 Hz, CH), 5.55 (dd, 1H, J=6.4, 4.8 Hz, CH), 7.39 (t, 2H, J=8.0 Hz, ArH), 7.45 (t, 2H, J=8.0 Hz, ArH), 7.51-7.56 (m, 1H, ArH), 7.57-7.61 (m, 1H, ArH), 8.05-8.07 (m, 4H, ArH); ¹³C NMR (100 MHz, CDCl₃): δ 55.4, 58.5, 64.7, 73.6, 75.3, 78.7, 78.9, 80.4, 106.8, 128.3, 128.5, 129.3, 129.73, 129.76, 129.84, 133.1, 133.4, 165.8, 166.2.

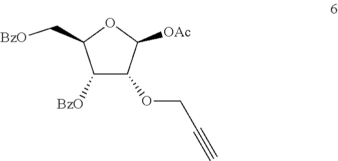

General procedure for the synthesis of (2R,3R,4R,5S)-5-acetoxy-2-((benzoyloxy)methyl)-4-(prop-2-yn-1-yloxy)tetrahyd-rofuran-3-yl benzoate (6): Compound (5) (534 mg, 1.3 mmol) was dissolved in a mixture of TFA/H₂O (9/1, 15 mL) and the resulting mixture was stirred at room temperature until the reaction complete (monitoring by TLC). Then the reaction was diluted with DCM (60 mL) and the solution was added dropwise to a stirred mixture of ice and saturated aqueous NaHCO₃. Solid NaHCO₃ was added during the addition to maintain a PH of 7. The mixture was extracted with DCM (3×50 mL), and the combined organic extracts was washed with H₂O (50 mL), brine (50 mL), dried over anhydrous Na₂SO₄, filtered, concentrated to give a residue. The residue was dissolved in pyridine (15 mL) and cooled to 0° C. Ac₂O (0.5 mL) was added dropwise and then the resulting mixture was allowed to warm to room temperature. After stirring for 6 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the compound (6) (445 mg, 78%) as a colorless oil.

(2R,3R,4R,5S)-5-acetoxy-2-((benzoyloxy)methyl)-4-(prop-2-yn-1-yloxy)tetrahyd-rofuran-3-yl benzoate (6). ¹H NMR (400 MHz, CDCl₃): δ 1.98 (s, 3H, CH₃), 2.39 (t, 1H, J=2.4 Hz, CH), 4.23-4.32 (m, 2H, CH₂), 4.43-4.48 (m, 1H, CH₂), 4.51 (d, 1H, J=5.2 Hz, CH), 4.67-4.73 (m, 2H, CH+CH₂), 4.53 (dd, 1H, J=6.8, 4.8 Hz, CH), 6.32 (s, 1H, CH), 7.40 (t, 2H, J=8.0 Hz, ArH), 7.46 (t, 2H, J=8.0 Hz, ArH), 7.55 (t, 1H, J=8.0 Hz, ArH), 7.58-7.62 (m, 1H, ArH), 8.05-8.07 (m, 4H, ArH); ¹³C NMR (100 MHz, CDCl₃): δ 21.0, 58.5, 63.7, 72.3, 75.6, 78.5, 79.7, 79.8, 99.0, 128.4, 128.5, 129.0, 129.6, 129.8, 129.9, 133.2, 133.6, 165.9, 166.0, 169.5.

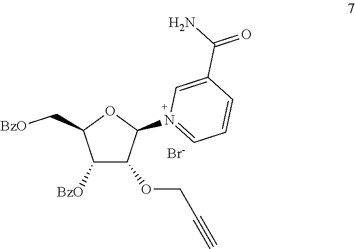

General procedure for the synthesis of 1-((2R,3R,4R,5R)-4-(benzoyloxy)-5-((benzoyloxy)methyl)-3-(prop-2-yn-1-yloxy)t-etrahydrofuran-2-yl)-3-carbamoyl-pyridin-1-ium bromide (7): Compound (6) (307 mg, 0.70 mmol) was dissolved in toluene (10 mL) and cooled to 0° C. HBr (33 wt % in acetic acid) (257 mg, 1.05 mmol, 1.5 eq) was added dropwise and the reaction was stirred at 0° C. for 5 hours. After the starting material was consumed, the reaction was concentrated under reduced pressure to give a residue. The residue was azeotroped with toluene (3×20 mL) to remove remaining acetic acid and dried in vacuo. The crude product and nicotinamide (103 mg, 0.84 mmol, 1.2 eq) was dissolved in CH₃CN (20 mL). The reaction was stirred under Ar gas at room temperature for 24 hours. The reaction was concentrated in vacuo (the temperature was kept below 35° C.) and purified by a flash column chromatography on silica gel to afford the compound (7) (277 mg, 68%) as a colorless solid.

1-((2R,3R,4R,5R)-4-(benzoyloxy)-5-((benzoyloxy)methyl)-3-(prop-2-yn-1-yloxy)t-etrahydrofuran-2-yl)-3-carbamoylpyridin-1-ium bromide (7). ¹H NMR (400 MHz, CDCl₃, TMS): δ 2.45 (t, 1H, J=2.0 Hz, CH), 4.28 (dd, 1H, J=16.0, 2.0 Hz, CH₂), 4.39 (dd, 1H, J=16.0, 2.0 Hz, CH₂), 4.66 (d, 2H, J=3.6 Hz, CH₂), 5.25 (t, 1H, J=5.2 Hz, CH), 5.51 (m, 1H, CH), 5.80 (dd, 1H, J=5.2, 2.0 Hz, CH), 6.15 (s, 1H, NH), 7.31 (d, 1H, J=5.2 Hz, CH), 7.37 (t, 2H, J=8.0 Hz, ArH), 7.51-7.58 (m, 3H, ArH), 7.60-7.64 (m, 1H, ArH), 7.66-7.68 (m, 2H, ArH), 8.03 (m, 1H, ArH), 8.09-8.11 (m, 2H, ArH), 9.14 (d, 1H, J=6.0 Hz, ArH), 9.22 (d, 1H, J=7.2 Hz, ArH), 9.31 (s, 1H, ArH), 10.45 (s, 1H, ArH); ¹³C NMR (100 MHz, CDCl₃, TMS): δ 59.9, 63.8, 71.5, 77.4, 77.5, 77.6, 84.8, 95.1, 126.6, 128.1, 128.7, 128.81, 128.83, 129.6, 129.9, 132.8, 133.7, 134.1, 141.8, 143.0, 146.7, 162.7, 165.0, 165.9.

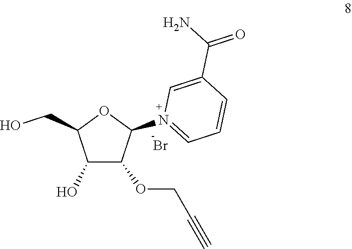

General procedure for the synthesis of 3-carbamoyl-1-((2R,3R,4R,5R)-4-hydroxy-5-(hydroxymethyl)-3-(prop-2-yn-1-yloxy)tetrahydrofuran-2-yl)pyridin-1-ium bromide (8, NR1): Compound (7) (260 mg, 0.45 mmol) was dissolved in ammonia (18 mL, 7 N in MeOH) and the reaction was stirred at −10° C. for 48 hours. The reaction was concentrated under reduced pressure and the crude product was dissolved in MeOH (0.5 mL). Addition of ethyl ether (10 mL) resulted in ppt of the desired product. The procedure was repeated three times to yield the desired product 8 (also referred to as NR1) (121 mg, 72%) as a colorless solid.

3-carbamoyl-1-((2R,3R,4R,5R)-4-hydroxy-5-(hydroxymethyl)-3-(prop-2-yn-1-ylo-xy)tetrahydrofuran-2-yl)pyridin-1-ium bromide (8, NR1). ¹H NMR (400 MHz, D₂O): δ 2.94 (t, 1H, J=2.4 Hz, CH), 3.79 (dd, 1H, J=13.2, 4.4 Hz, CH₂), 3.91 (dd, 1H, J=13.2, 2.8 Hz, CH₂), 4.31 (dd, 1H, J=16.0, 2.4 Hz, CH₂), 4.38 (dd, 1H, J=16.0, 2.4 Hz, CH₂), 4.56-4.58 (m, 1H, CH), 4.83 (dd, 1H, J=6.4, 3.2 Hz, CH), 4.88 (t, 1H, J=5.2 Hz, CH), 6.67 (d, 1H, J=5.2 Hz, CH), 8.20-8.24 (m, 1H, ArH), 8.96 (d, 1H, J=7.6 Hz, ArH), 9.12 (d, 1H, J=6.8 Hz, ArH), 9.33 (s, 1H, ArH); ¹³C NMR (100 MHz, D₂O): δ 59.0, 60.9, 69.3, 76.9, 78.5, 78.7, 89.4, 95.4, 126.9, 132.4, 141.2, 143.7, 145.2, 165.8; HRMS (ESI) Calcd. For C₁₄H₁₇N₂O₅⁺ (M⁺) requires 293.1132, Found: 293.1134.

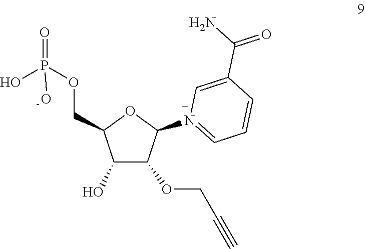

General procedure for the synthesis of ((2R,3R,4R,5R)-5-(3-carbamoylpyridin-1-ium-1-yl)-3-hydroxy-4-(prop-2-yn-1-yloxy)tetrahydrofuran-2-yl)methyl hydro-gen phosphate (9, NM1): To a stirred solution of compound (8, NR1) (100 mg, 0.27 mmol) in trimethylphosphate (2 mL) was added P(O)Cl₃ (175 μL, 1.89 mmol, 7 eq) at 0° C. and the resulting mixture was stirred at 0° C. for 6 hours. A few drops H₂O was then added to quench the reaction. Trimethylphosphate was removed by extraction with ethyl ether (3×20 ml). The remaining trimethylphosphate was removed by a second extraction with THF (5 ml). The aqueous layer was concentrated in vacuo and the crude product was dissolved in MeOH (0.5 mL). Addition of ethyl ether (10 mL) resulted in ppt of the desired product. The procedure was repeated three times to yield the desired product 9 (also referred to as NM1) (60 mg, 60%) as a colorless solid.

((2R,3R,4R,5R)-5-(3-carbamoylpyridin-1-ium-1-yl)-3-hydroxy-4-(prop-2-yn-1-yloxy)tetrahydrofuran-2-yl)methyl hydrogen phosphate (9). ¹H NMR (400 MHz, D₂O): δ 2.87 (t, 1H, J=2.4 Hz, CH), 4.04-4.10 (m, 1H, CH₂), 4.13-4.18 (m, 1H, CH₂), 4.26 (dd, 1H, J=16.4, 2.4 Hz, CH₂), 4.32 (dd, 1H, J=16.4, 2.4 Hz, CH₂), 4.60 (dd, 1H, J=4.8, 1.6 Hz, CH), 4.86 (t, 1H, J=1.6 Hz, CH), 4.89-4.91 (m, 1H, CH), 6.62 (d, 1H, J=6.0 Hz, CH), 8.15 (m, 1H, ArH), 8.90 (d, 1H, J=8.0 Hz, ArH), 9.06 (d, 1H, J=6.4 Hz, ArH), 9.28 (s, 1H, ArH); ¹³C NMR (100 MHz, D₂O): δ 59.0, 64.8 (d, J=4.5 Hz), 69.3, 76.8, 78.5, 78.7, 87.8 (d, J=8.5 Hz), 95.5, 126.9, 132.4, 141.2, 143.7, 145.2, 165.8; HRMS (ESI) Calcd. For C₁₄H₁₈N₂O₈P⁺ (M+H)⁺ requires 373.0795, Found: 373.0788.

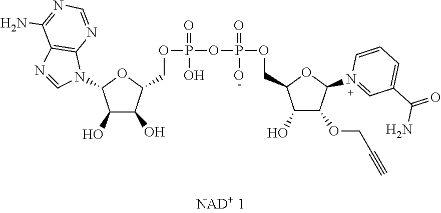

General procedure for the synthesis of 1-((2R,3R,4R,5R)-5-((((((((2R,3S,4R,5R)-5-(6-amino-9H-purin-9-yl)-3,4-dihydr-oxytetrahydrofuran-2-yl)methoxy)(hydroxy)p-hosphoryl)oxy)oxidophosphoryl)o-xy)methyl)-4-hydroxy-3-(prop-2-yn-1-yloxy)tet-rahydrofuran-2-yl)-3-carbamoy-lpyridin-1-ium (10, NAD⁺1): To a stirred solution of Adenosine 5′-monophosphate (5′-AMP) (52 mg, 0.15 mmol, 1.5 eq) in dried DMF (2 mL) were added 1,1-carbonyldiimidazole (CDI) (63 mg, 0.50 mmol, 5 eq) and triethylamine (23 μL, 0.16 mmol. 1.6 eq). The reaction mixture was stirred at room temperature for 14 hours, and then quenched with 0.100 ml dried methanol. The solvent was removed under vacuum and the residue was coevaporated 3 times each with 1.00 ml of dried DMF. The activated 5′-AMP was dissolved in dried DMF (1 mL) and compound (9, NM1) (37 mg, 0.10 mmol, 1.0 eq) was added. After stirring at room temperature for 4 days, H₂O was added to quench the reaction at 0° C. The resulting mixture was continued stirring at room temperature for 24 hours. The reaction was then concentrated in vacuo and the crude product was purified via preparative HPLC (C18-A column, 150×4.6 mm, 5 m) (mobile phase A: 0.1% formic acid (aq), mobile B: 0.1% formic acid in acetonitrile; flow rate=1.0 ml/min; 0-16 min: 0-6.7% B, 16-18 min: 6.7-0% B). Fractions containing the desired product were concentrated and lyophilized to yield NAD⁺1 (32 mg, 45% yield) as a colorless solid.

1-((2R,3R,4R,5R)-5-((((((((2R,3 S,4R,5R)-5-(6-amino-9H-purin-9-yl)-3,4-dihydr-oxytetrahydrofuran-2-yl)methoxy)(hydroxy)phosphoryl)oxy)oxidophosphoryl)o-xy)methyl)-4-hydroxy-3-(prop-2-yn-1-yloxy)tetrahydrofuran-2-yl)-3-carbamoy-lpyridin-1-ium (10, NAD⁺1). ¹H NMR (400 MHz, D₂O): δ 2.87 (br, 1H, CH), 4.12-4.14 (m, 1H, CH), 4.24-4.33 (m, 5H, 2CH₂+CH), 4.40 (br, 1H, CH), 4.51 (t, 1H, J=4.0 Hz, CH₂), 4.63 (d, 1H, J=2.8 Hz, CH), 4.71 (t, 1H, J=5.2 Hz, CH), 4.87-4.89 (m, 2H, 2CH), 6.06 (d, 1H, J=5.2 Hz, CH), 6.59 (d, 1H, J=5.6 Hz, CH), 8.09-8.13 (m, 1H, ArH), 8.31 (br, 1H, ArH), 8.61 (br, 1H, ArH), 8.86 (d, 1H, J=8.0 Hz, ArH), 8.01 (d, 1H, J=6.0 Hz, ArH), 9.19 (s, 1H, ArH); ¹³C NMR (100 MHz, D₂O): δ 59.0, 65.2, 69.3, 70.2, 74.6, 76.7, 78.5, 78.7, 84.1, 87.3, 87.9, 95.6, 126.8, 132.1, 140.9, 143.7, 145.0, 147.6, 148.5, 151.8, 165.2; HRMS (ESI) Calcd. For C₂₄H₂₈N₇Na₂O₁₄P₂⁺ (M+H)⁺ requires 746.0965, Found: 746.0955.

### Example 2. Synthesis of NAD⁺2-6 and 21

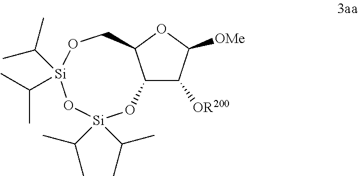

General procedure for the synthesis of compound 3aa: To a stirred solution of (6aR,8R, 9R,9aS)-2,2,4,4-tetraisopropyl-8-methoxytetrahydro-6H-furo[3,2-f][1,3,5,2,4]trioxadisilocin-9-ol in anhydrous THF was added NaH at 0° C. followed by the addition of corresponding R²⁰⁰Br or R²⁰⁰OTf (R²⁰⁰ may be —(CH₂)ₙC ≡CH wherein n is 1, 2, or 3) at the same temperature. Then reaction mixture was allowed to warm to room temperature. After stirring at this temperature for 6 hours, the reaction mixture was quenched with saturated aqueous NH₄Cl (20 mL) and extracted with EtOAc (3×50 mL). The combined organic layers were washed water (3×50 mL), dried over anhydrous Na₂SO₄, filtered and concentrated and purified by a flash column chromatography on silica gel to afford the corresponding compound 3aa.

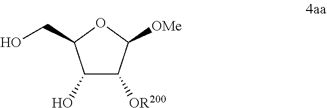

General procedure for the synthesis of compound 4aa: To a 0° C. solution of compound 3aa (2.1 mmol) in anhydrous THF (25 mL) was added AcOH (3.2 mmol, 1.5 eq) followed by the addition of TBAF (3.2 mL, 3.2 mmol, 1.0 M in THF, 1.5 eq). Then reaction mixture was allowed to warm to room temperature. After stirring at this temperature for 14 hours, the reaction mixture was concentrated under reduced pressure to give a residue. The residue was purified by a flash column chromatography on silica gel to afford the compound 4aa.

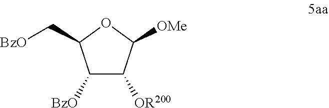

General procedure for the synthesis of compound 5aa: To a solution of compound 4aa (1.7 mmol) in a mixture of anhydrous DCM (10 mL) and anhydrous pyridine (10 mL) was added BzCl (588 μL, 5.1 mmol, 3 eq) at 0° C. Then the reaction mixture was allowed to warm to room temperature. After stirring for 24 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the corresponding compound 5aa.

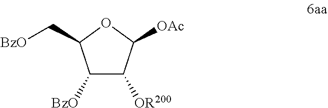

General procedure for the synthesis of compound 6aa: Compound 5aa (1.3 mmol) was dissolved in a mixture of TFA/H₂O (9/1, 15 mL) and the resulting mixture was stirred at room temperature until the reaction complete (monitoring by TLC). Then the reaction was diluted with DCM (60 mL) and the solution was added dropwise to a stirred mixture of ice and saturated aqueous NaHCO₃. Solid NaHCO₃ was added during the addition to maintain a PH of 7. The mixture was extracted with DCM (3×50 mL), and the combined organic extracts was washed with H₂O (50 mL), brine (50 mL), dried over anhydrous Na₂SO₄, filtered, concentrated to give a residue. The residue was dissolved in pyridine (15 mL) and cooled to 0° C. Ac₂O (0.5 mL) was added dropwise and then the resulting mixture was allowed to warm to room temperature. After stirring for 6 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the corresponding compound 6aa.

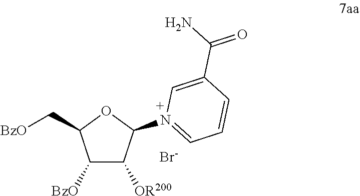

General procedure for the synthesis of compound 7aa: Compound 6aa (0.70 mmol) was dissolved in toluene (10 mL) and cooled to 0° C. HBr (33 wt % in acetic acid) (257 mg, 1.05 mmol, 1.5 eq) was added dropwise and the reaction was stirred at 0° C. for 5 hours. After the starting material was consumed, the reaction was concentrated under reduced pressure to give a residue. The residue was azeotroped with toluene (3×20 mL) to remove remaining acetic acid and dried in vacuo. The crude product and nicotinamide (103 mg, 0.84 mmol, 1.2 eq) was dissolved in CH₃CN (20 mL). The reaction was stirred under Ar gas at room temperature for 24 hours. The reaction was concentrated in vacuo (the temperature was kept below 35° C.) and purified by a flash column chromatography on silica gel to afford the corresponding compound 7aa.

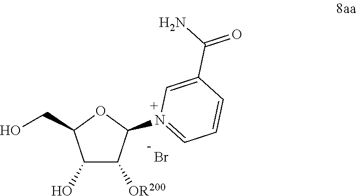

General procedure for the synthesis of compound 8aa: Compound 7aa (0.45 mmol) was dissolved in ammonia (18 mL, 7 N in MeOH) and the reaction was stirred at −10° C. for 48 hours. The reaction was concentrated under reduced pressure and the crude product was dissolved in MeOH (0.5 mL). Addition of ethyl ether (10 mL) resulted in ppt of the desired product. The procedure was repeated three times to yield the corresponding desired product 8aa.

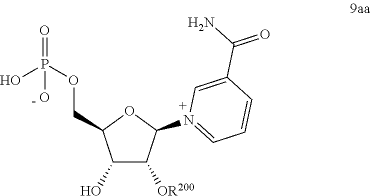

General procedure for the synthesis of compound 9aa: To a stirred solution of compound 8aa (0.27 mmol) in trimethylphosphate (2 mL) was added P(O)Cl₃ (175 μL, 1.89 mmol, 7 eq) at 0° C. and the resulting mixture was stirred at 0° C. for 6 hours. A few drops H₂O was then added to quench the reaction. Trimethylphosphate was removed by extraction with ethyl ether (3×20 ml). The remaining trimethylphosphate was removed by a second extraction with THF (5 ml). The aqueous layer was concentrated in vacuo and the crude product was dissolved in MeOH (0.5 mL). Addition of ethyl ether (10 mL) resulted in ppt of the desired product. The procedure was repeated three times to yield the corresponding desired product 9aa.

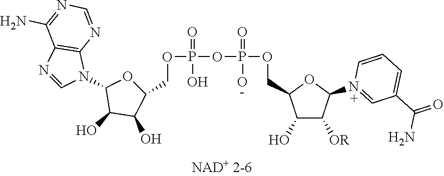

General procedure for the synthesis of NAD⁺3-6 and NAD⁺21: To a stirred solution of Adenosine 5′-monophosphate (5′-AMP) (52 mg, 0.15 mmol, 1.5 eq) in dried DMF (2 mL) were added 1,1-carbonyldiimidazole (CDI) (63 mg, 0.50 mmol, 5 eq) and triethylamine (23 μL, 0.16 mmol. 1.6 eq). The reaction mixture was stirred at room temperature for 14 hours, and then quenched with 0.100 ml dried methanol. The solvent was removed under vacuum and the residue was coevaporated 3 times each with 1.00 ml of dried DMF. The activated 5′-AMP was dissolved in dried DMF (1 mL) and compound 9aa (0.10 mmol, 1.0 eq) was added. After stirring at room temperature for 4 days, H₂O was added to quench the reaction at 0° C. The resulting mixture was continued stirring at room temperature for 24 hours. The reaction was then concentrated in vacuo and the crude product was purified via preparative HPLC (C18-A column, 150×4.6 mm, 5 μm) (mobile phase A: 0.1% formic acid (aq), mobile B: 0.1% formic acid in acetonitrile; flow rate=1.0 ml/min; 0-16 min: 0-6.7% B, 16-18 min: 6.7-0% B). Fractions containing the desired product were concentrated and lyophilized to yield corresponding NAD⁺3-6 and NAD⁺21.

NAD⁺2 is prepared following the procedure as described above with the necessary modifications well-understood by the skilled artisan.

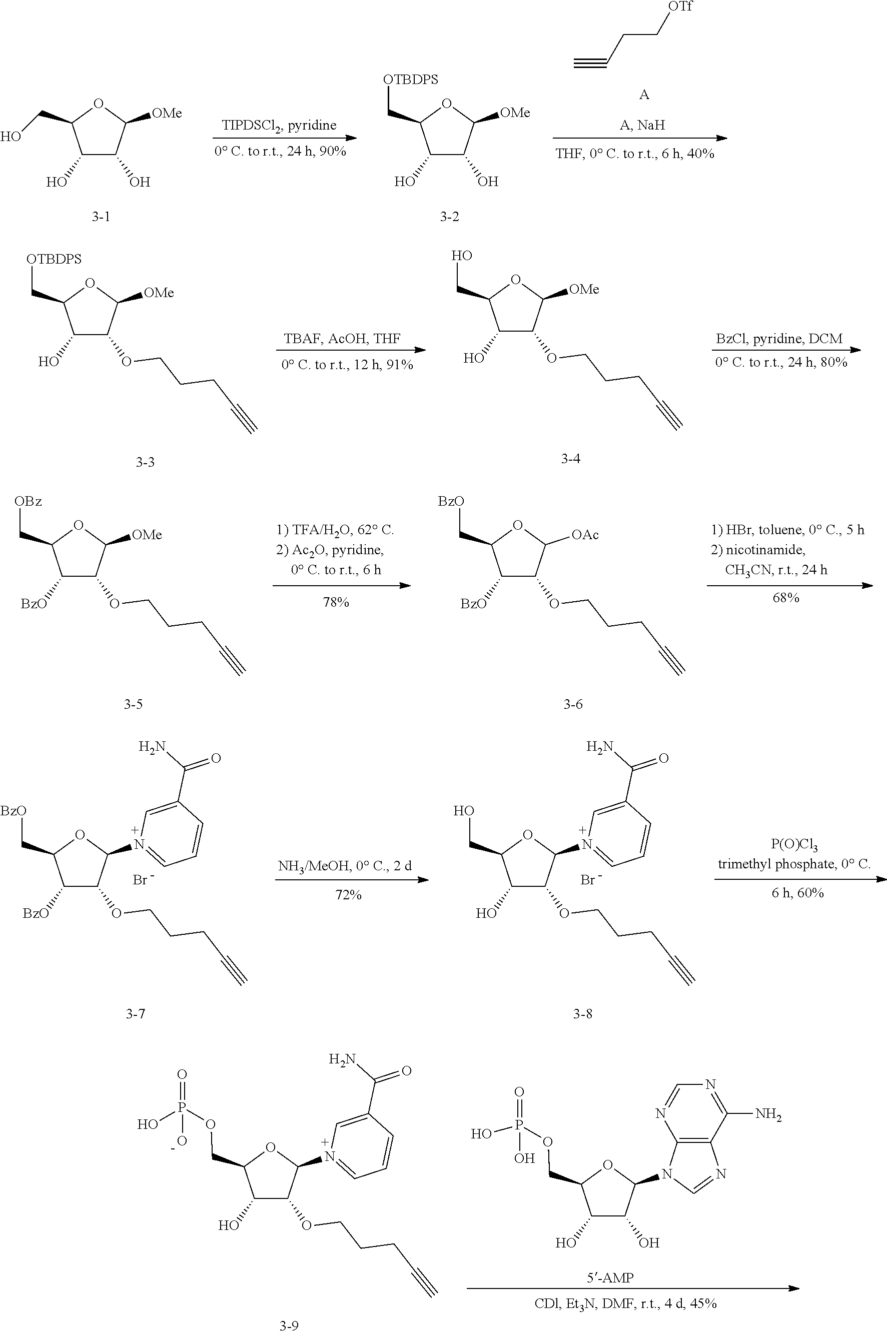

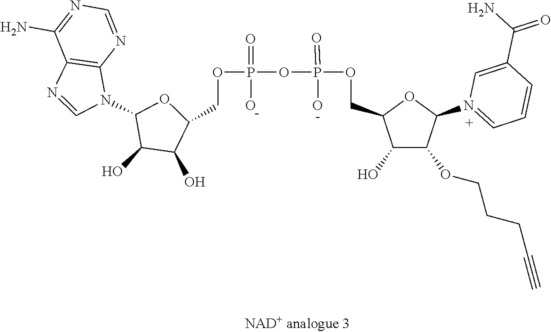

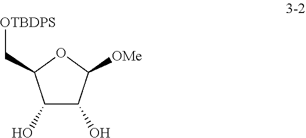

General procedure for the synthesis of (2R,3 S,4R,5R)-2-(((tert-butyldiphenylsilyl)oxy)methyl)-5-methoxytetrahydrofuran-3,4-diol (3-2): To a stirred solution of Methyl-β-D-Ribofuranoside (3-1) (1.64 g, 10.0 mmol) and imidazole (1.36 g, 20.0 mmol, 2.0 eq) in anhydrous DMF (20 mL) was added TBDPSCl (3.02 g, 11.0 mmol, 1.1 eq) at 0° C. Then reaction mixture was allowed to warm to room temperature. After stirring at this temperature for 24 h, the reaction mixture was diluted with EtOAc (100 mL), and the organic phase was washed successively with ice-water (50 mL), aq 1M HCl (2×50 mL), H₂O (2×50 mL), dried over anhydrous Na₂SO₄, filtered and concentrated to give a residue. The residue was purified by a flash column chromatography on silica gel to afford the compound (3-2) (3.62 g, 90%) as a colorless oil.

(2R,3S,4R,5R)-2-(((tert-butyldiphenylsilyl)oxy)methyl)-5-methoxytetrahydrofuran-3,4-diol (3-2). A colorless oil, 90% yield; ¹H NMR (400 MHz, CDCl₃): δ 1.07 (s, 9H, 3CH₃), 2.30 (d, 1H, J=5.6 Hz, OH), 2.62 (d, 1H, J=3.2 Hz, OH), 3.31 (s, 3H, OCH₃), 3.77 (dd, 1H, J=10.8, 6.0 Hz, CH₂), 3.83 (dd, 1H, J=10.8, 4.4 Hz, CH₂), 4.01-4.05 (m, 2H, 2CH), 4.32-4.36 (m, 1H, CH), 4.84 (s, 1H, CH), 7.37-7.46 (m, 6H, ArH), 7.68-7.70 (m, 4H, ArH); ¹³C NMR (100 MHz, CDCl₃): δ 19.2, 26.8, 55.2, 65.1, 72.7, 75.3, 82.9, 108.1, 127.75, 127.78, 129.80, 129.83, 133.2, 135.6.

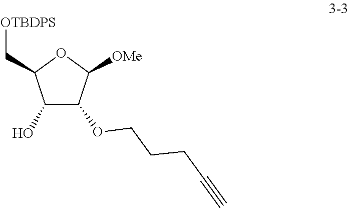

General procedure for the synthesis of compound (3-3): To a stirred solution of (2R,3 S,4R,5R)-2-(((tert-butyldiphenylsilyl)oxy)methyl)-5-methoxytetrahydrofuran-3,4-diol (3-2) (3.62 g, 9.0 mmol) in anhydrous THF (30 mL) was added NaH (432 mg, 10.8 mmol, 1.2 eq, 60% dispersion in mineral oil) at 0° C. followed by the addition of pent-4-yn-1-Y¹ trifluoromethanesulfonate (2.92 g, 13.5 mmol, 1.5 eq) at the same temperature. Then the reaction mixture was allowed to warm to room temperature. After stirring at this temperature for 8 hours, the reaction mixture was quenched with saturated aqueous NH₄Cl (20 mL) and extracted with EtOAc (3×50 mL). The combined organic layers were washed water (3×50 mL), dried over anhydrous Na₂SO₄, filtered and concentrated and purified by a flash column chromatography on silica gel to afford the compound (3-3) (1.69 g, 40%) as a colorless oil.

(2R,3R,4R,5R)-2-(((tert-butyldiphenyl silyl)oxy)methyl)-5-methoxy-4-(pent-4-yn-1-yloxy)tet-rahydrofuran-3-ol (3-3). A colorless oil, 40% yield; ¹H NMR (400 MHz, CDCl₃): δ 1.07 (s, 9H, 3CH₃), 1.81-1.88 (m, 2H, CH₂), 1.99 (t, 1H, J=2.8 Hz, CH), 2.30-2.36 (m, 2H, CH₂), 2.56 (d, 1H, J=8.0 Hz, OH), 3.35 (s, 3H, OCH₃), 3.65-3.85 (m, 5H, 2CH₂+CH), 4.01 (dd, 1H, J=10.0, 4.0 Hz, CH), 4.28-4.33 (m, 1H, CH), 4.91 (d, 1H, J=0.8 Hz, CH), 7.36-7.45 (m, 6H, ArH), 7.70-7.73 (m, 4H, ArH); ¹³C NMR (100 MHz, CDCl₃): δ 15.2, 19.3, 26.8, 28.3, 55.3, 64.6, 69.0, 69.2, 71.0, 82.7, 83.5, 84.6, 105.8, 127.66, 127.68, 129.63, 129.67, 133.39, 133.41, 135.6.

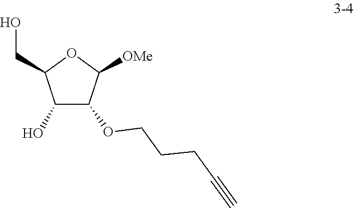 [SMILES: C#CCCCO[C@H]1[C@H](OC)O[C@H](CO)[C@H]1O]

General procedure for the synthesis of (2R,3R,4R,5R)-2-(hydroxymethyl)-5-methoxy-4-(pent-4-yn-1-yloxy)tetrahydrofuran-3-ol (3-4): To a 0° C. solution of (2R,3R,4R,5R)-2-(((tert-butyldiphenylsilyl)oxy)methyl)-5-methoxy-4-(pent-4-yn-1-yloxy)tet-rahydrofuran-3-ol (3-3) (1.17 g, 2.50 mmol) in anhydrous THF (25 mL) was added AcOH (225 mg, 3.75 mmol, 1.5 eq) followed by the addition of TBAF (3.75 mL, 3.75 mmol, 1.0 M in THF, 1.5 eq). Then the reaction mixture was allowed to warm to room temperature. After stirring at this temperature for 14 hours, the reaction mixture was concentrated under reduced pressure to give a residue. The residue was purified by a flash column chromatography on silica gel to afford the compound (2R,3R,4R,5R)-2-(hydroxymethyl)-5-methoxy-4-(pent-4-yn-1-yloxy)tetrahydrofuran-3-ol (3-4) (524 mg, 91%) as a colorless oil.

(2R,3R,4R,5R)-2-(hydroxymethyl)-5-methoxy-4-(pent-4-yn-1-yloxy)tetrahydrofuran-3-ol (3-4). A colorless oil, 91% yield; ¹H NMR (400 MHz, CDCl₃): δ 1.78-1.85 (m, 2H, CH₂), 1.98 (t, 1H, J=2.4 Hz, CH), 2.28-2.32 (m, 2H, CH₂), 2.77 (d, 1H, J=8.0 Hz, OH), 3.40 (s, 3H, OCH₃), 3.58-3.69 (m, 2H, CH₂), 3.73-3.79 (m, 3H, CH₂+CH), 4.01-4.04 (m, 1H, CH), 4.22-4.27 (m, 1H, CH), 4.88 (d, 1H, J=1.2 Hz, CH); ¹³C NMR (100 MHz, CDCl₃): δ 15.2, 28.2, 55.7, 63.1, 69.1, 69.3, 70.9, 83.1, 83.4, 85.6, 106.5.

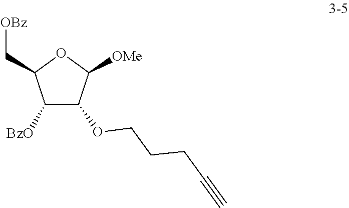

General procedure for the synthesis of compound ((2R,3R,4R,5R)-3-(benzoyloxy)-5-methoxy-4-(pent-4-yn-1-yloxy)tetrahydrofuran-2-yl)methyl benzoate (3-5): To a solution of (2R,3R,4R,5R)-2-(hydroxymethyl)-5-methoxy-4-(pent-4-yn-1-yloxy)tetrahydrofuran-3-ol (3-4) (460 mg, 2.0 mmol) in a mixture of anhydrous DCM (10 mL) and anhydrous pyridine (10 mL) was added BzCl (691 μL, 6.0 mmol, 3 eq) at 0° C. Then the reaction mixture was allowed to warm to room temperature. After stirring for 24 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the corresponding compound ((2R,3R,4R,5R)-3-(benzoyloxy)-5-methoxy-4-(pent-4-yn-1-yloxy)tetrahydrofuran-2-yl)methyl benzoate (3-5) (702 mg, 80%) as a colorless oil.

((2R,3R,4R,5R)-3-(benzoyloxy)-5-methoxy-4-(pent-4-yn-1-yloxy)tetrahydrofuran-2-yl)met-hyl benzoate (3-5). A colorless oil, 80% yield; ¹H NMR (400 MHz, CDCl₃): δ 1.68-1.75 (m, 2H, CH₂), 1.84 (t, 1H, J=2.4 Hz, CH), 2.12-2.23 (m, 2H, CH₂), 3.37 (s, 3H, OCH₃), 3.57-3.62 (m, 1H, CH₂), 3.65-3.70 (m, 1H, CH₂), 4.17 (d, 1H, J=4.8 Hz, CH), 4.45 (dd, 1H, J=11.6, 4.8 Hz, CH₂), 4.58-4.67 (m, 2H, CH₂+CH), 4.98 (s, 1H, CH), 5.48 (dd, 1H, J=6.6, 4.8 Hz, CH), 7.37-7.41 (m, 2H, ArH), 7.43-7.47 (m, 2H, ArH), 7.52-7.60 (m, 2H, ArH), 8.04-8.05 (m, 2H, ArH), 8.06-8.07 (m, 2H, ArH); ¹³C NMR (100 MHz, CDCl₃): δ 14.9, 28.6, 55.2, 64.8, 68.6, 69.4, 73.9, 78.6, 81.3, 83.5, 106.8, 128.3, 128.5, 129.4, 129.7, 129.8, 133.1, 133.4, 165.9, 166.3.

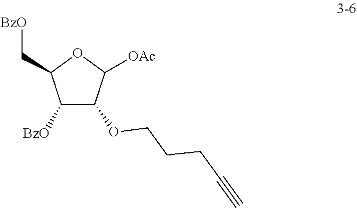

General procedure for the synthesis of (2R,3R,4R)-5-acetoxy-2-((benzoyloxy)methyl)-4-(pent-4-yn-1-yloxy)tetrahydrofuran-3-yl benzoate (3-6): Compound (3-5) (570 mg, 1.3 mmol) was dissolved in a mixture of TFA/H₂O (9/1, 15 mL) and the resulting mixture was stirred at room temperature until the reaction complete (monitoring by TLC). Then the reaction was diluted with DCM (60 mL) and the solution was added dropwise to a stirred mixture of ice and saturated aqueous NaHCO₃. Solid NaHCO₃ was added during the addition to maintain a pH of 7. The mixture was extracted with DCM (3×50 mL), and the combined organic extracts was washed with H₂O (50 mL), brine (50 mL), dried over anhydrous Na₂SO₄, filtered, concentrated to give a residue. The residue was dissolved in pyridine (15 mL) and cooled to 0° C. Ac₂O (0.5 mL) was added dropwise and then the resulting mixture was allowed to warm to room temperature. After stirring for 6 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the corresponding compound (2R,3R,4R)-5-acetoxy-2-((benzoyloxy)methyl)-4-(pent-4-yn-1-yloxy)tetrahydrofuran-3-yl benzoate (3-6) (473 mg, 78%) as a colorless oil.

(2R,3R,4R)-5-acetoxy-2-((benzoyloxy)methyl)-4-(pent-4-yn-1-yloxy)tetrahydrofuran-3-yl b-enzoate (3-6). A colorless solid, 78% yield; ¹H NMR (400 MHz, CDCl₃): δ 1.68-1.74 (m, 2H, CH₂), 1.84 (t, 1H, J=2.8 Hz, CH), 1.97 (s, 3H, CH₃), 2.14-2.19 (m, 2H, CH₂), 3.57-3.62 (m, 1H, CH₂), 3.71-3.76 (m, 1H, CH₂), 4.27 (d, 1H, J=5.2 Hz, CH), 4.42-4.47 (m, 1H, CH₂), 4.68-4.74 (m, 2H, CH₂+CH), 5.45 (dd, 1H, J=7.2, 4.8 Hz, CH), 6.23 (s, 1H, CH), 7.37-7.41 (m, 2H, ArH), 7.44-7.47 (m, 2H, ArH), 7.52-7.56 (m, 1H, ArH), 7.58-7.62 (m, 1H, ArH), 8.04-8.07 (m, 4H, ArH); ¹³C NMR (100 MHz, CDCl₃): δ 14.9, 21.0, 28.4, 63.7, 68.7, 69.5, 72.6, 79.5, 80.7, 83.4, 99.1, 128.4, 128.5, 129.0, 129.6, 129.7, 129.8, 133.2, 133.6, 166.96, 166.05, 169.6.

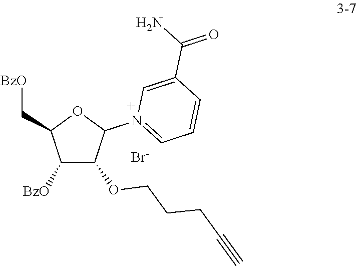

General procedure for the synthesis of 1-((2R,3R,4R,5R)-4-(benzoyloxy)-5-((benzoyloxy)methyl)-3-(pent-4-yn-1-yloxy)tetrahydrofuran-2-yl)-3-carbamoylpyridin-1-ium bromide (3-7): Compound (3-6) (327 mg, 0.70 mmol) was dissolved in toluene (10 mL) and cooled to 0° C. HBr (33 wt % in acetic acid) (257 mg, 1.05 mmol, 1.5 eq) was added dropwise and the reaction was stirred at 0° C. for 5 hours. After the starting material was consumed, the reaction was concentrated under reduced pressure to give a residue. The residue was azeotroped with toluene (3×20 mL) to remove remaining acetic acid and dried in vacuo. The crude product and nicotinamide (103 mg, 0.84 mmol, 1.2 eq) was dissolved in CH₃CN (20 mL). The reaction was stirred under Ar gas at room temperature for 24 hours. The reaction was concentrated in vacuo (the temperature was kept below 35° C.) and purified by a flash column chromatography on silica gel to afford the corresponding compound 1-((2R,3R,4R,5R)-4-(benzoyloxy)-5-((benzoyloxy)methyl)-3-(pent-4-yn-1-yloxy)tetrahydrofuran-2-yl)-3-carbamoylpyridin-1-ium bromide (3-7) (290 mg 68%) as a colorless solid.

1-((2R,3R,4R,5R)-4-(benzoyloxy)-5-((benzoyloxy)methyl)-3-(pent-4-yn-1-yloxy)tetrahydrof-uran-2-yl)-3-carbamoylpyridin-1-ium bromide (3-7). A colorless solid, 68% yield; ¹H NMR (400 MHz, CD₃OD): δ 1.53-1.67 (m, 2H, CH₂), 1.92-1.96 (m, 2H, CH₂), 2.09 (t, 1H, J=2.4 Hz, CH), 3.75-3.86 (m, 2H, CH₂), 4.73 (d, 2H, J=4.8 Hz, CH₂), 5.08 (t, 1H, J=6.0 Hz, CH), 5.56 (t, 1H, J=4.8 Hz, CH), 5.95 (dd, 1H, J=4.8, 0.8 Hz, CH), 6.87 (d, 1H, J=6.0 Hz, CH), 7.40-7.43 (m, 2H, ArH), 7.53-7.62 (m, 3H, ArH), 7.66-7.69 (m, 3H, ArH), 8.12-8.14 (m, 2H, ArH), 8.26 (dd, 1H, J=8.0, 6.4 Hz, ArH), 9.04 (d, 1H, J=8.0 Hz, ArH), 9.28 (d, 1H, J=6.4 Hz, ArH), 9.53 (s, 1H, ArH); ¹³C NMR (100 MHz, CD₃OD): δ 14.3, 28.1, 63.6, 68.8, 70.7, 71.1, 78.9, 82.3, 85.2, 95.1, 126.6, 128.36, 128.46, 128.55, 129.0, 129.32, 129.33, 133.1, 133.3, 133.5, 141.1, 143.6, 144.9, 163.4, 164.7, 166.0.

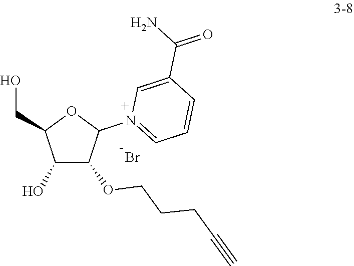

General procedure for the synthesis of 3-carbamoyl-1-((2R,3R,4R,5R)-4-hydroxy-5-(hydroxymethyl)-3-(pent-4-yn-1-yloxy)tetrahydrofuran-2-yl)pyridin-1-ium bromide (3-8): Compound (3-7) (274 mg, 0.45 mmol) was dissolved in ammonia (20 mL, 7 N in MeOH) and the reaction was stirred at 0° C. for 48 hours. The reaction was concentrated under reduced pressure and the crude product was dissolved in MeOH (0.5 mL). Addition of ethyl ether (10 mL) resulted in ppt of the desired product. The procedure was repeated three times to yield the corresponding desired product 3-carbamoyl-1-((2R,3R,4R,5R)-4-hydroxy-5-(hydroxymethyl)-3-(pent-4-yn-1-yloxy)tetrahydrofuran-2-yl)pyridin-1-ium bromide (3-8) (130 mg, 72%) as a colorless solid.

3-carbamoyl-1-((2R,3R,4R,5R)-4-hydroxy-5-(hydroxymethyl)-3-(pent-4-yn-1-yloxy)tetrahy-drofuran-2-yl)pyridin-1-ium bromide (3-8). A colorless solid, 72% yield; ¹H NMR (400 MHz, D₂O): δ 1.63-1.71 (m, 2H, CH₂), 2.02-2.17 (m, 2H, CH₂), 2.32-2.37 (m, 1H, CH), 3.67-3.81 (m, 3H, CH₂+CH₂), 3.91 (dd, 1H, J=12.8, 2.8 Hz, CH₂), 4.51-4.53 (m, 1H, CH), 4.70 (t, 1H, J=5.2 Hz, CH), 4.81-4.83 (m, 1H, CH), 6.87 (d, 1H, J=5.6 Hz, CH), 8.23-8.27 (m, 1H, ArH), 8.99 (d, 1H, J=7.6 Hz, ArH), 9.14 (d, 1H, J=5.6 Hz, ArH), 9.32 (s, 1H, ArH); ¹³C NMR (100 MHz, D₂O): δ 14.3, 27.4, 61.0, 69.3, 69.9, 70.4, 79.5, 84.6, 89.4, 95.5, 127.0, 132.3, 141.2, 143.5, 145.2, 165.7.

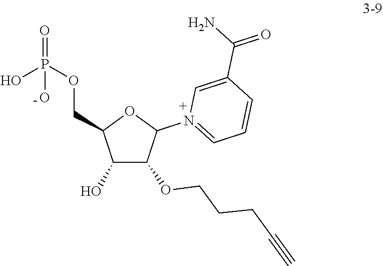

General procedure for the synthesis of ((2R,3R,4R,5R)-5-(3-carbamoylpyridin-1-ium-1-yl)-3-hydroxy-4-(pent-4-yn-1-yloxy)tetrahydrofuran-2-yl)methyl hydrogen phosphate (3-9): To a stirred solution of compound (3-8) (108 mg, 0.27 mmol) in trimethylphosphate (2 mL) was added P(O)Cl₃ (175 μL, 1.89 mmol, 7 eq) at 0° C. and the resulting mixture was stirred at 0° C. for 6 h. A few drops H₂O was then added to quench the reaction. Trimethylphosphate was removed by extraction with ethyl ether (3×20 ml). The remaining trimethylphosphate was removed by a second extraction with THF (5 ml). The aqueous layer was concentrated in vacuo and the crude product was dissolved in MeOH (0.5 mL). Addition of ethyl ether (10 mL) resulted in ppt of the desired product. The procedure was repeated three times to yield the corresponding desired product ((2R,3R,4R,5R)-5-(3-carbamoylpyridin-1-ium-1-yl)-3-hydroxy-4-(pent-4-yn-1-yloxy)tetrahydrofuran-2-yl)methyl hydrogen phosphate (3-9) (65 mg, 60%) as a colorless solid.

((2R,3R,4R,5R)-5-(3-carbamoylpyridin-1-ium-1-yl)-3-hydroxy-4-(pent-4-yn-1-yloxy)tetrah-ydrofuran-2-yl)methyl hydrogen phosphate (3-9). A colorless solid, 60% yield; ¹H NMR (400 MHz, D₂O): δ 1.63-1.69 (m, 2H, CH₂), 2.01-2.18 (m, 2H, CH₂), 2.32 (t, 1H, J=2.4 Hz, CH), 3.70-3.81 (m, 2H, CH₂), 4.04-4.09 (m, 1H, CH₂), 4.12-4.17 (m, 1H, CH₂), 4.59 (dd, 1H, J=4.4, 2.0 Hz, CH), 4.76 (t, 1H, J=6.0 Hz, CH), 4.93-4.95 (m, 1H, CH), 6.65 (d, 1H, J=6.0 Hz, CH), 8.21 (dd, 1H, J=8.0, 6.4 Hz, ArH), 8.96 (d, 1H, J=8.0 Hz, ArH), 9.12 (d, 1H, J=6.4 Hz, ArH), 9.32 (s, 1H, ArH); ¹³C NMR (100 MHz, D₂O): δ 14.2, 27.3, 64.6 (d, J=4.7 Hz), 69.3, 69.7, 70.3, 79.3, 84.6 (d, J=1.5 Hz), 88.3 (d, J=9.1 Hz), 95.6, 126.9, 132.3, 141.2, 143.5, 145.1, 165.8.

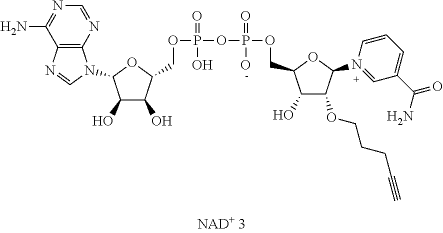

General procedure for the synthesis of (NAD⁺3): To a stirred solution of Adenosine 5′-monophosphate (5′-AMP) (52 mg, 0.15 mmol, 1.5 eq) in dried DMF (2 mL) were added 1,1-carbonyldiimidazole (CDI) (63 mg, 0.50 mmol, 5 eq) and triethylamine (23 μL, 0.16 mmol. 1.6 eq). The reaction mixture was stirred at room temperature for 14 h, and then quenched with 0.100 ml dried methanol. The solvent was removed under vacuum and the residue was coevaporated 3 times each with 1.00 ml of dried DMF. The activated 5′-AMP was dissolved in dried DMF (1 mL) and compound (3-9) (0.10 mmol, 1.0 eq) was added. After stirring at room temperature for 4 days, H₂O was added to quench the reaction at 0° C. The resulting mixture was continued stirring at room temperature for 24 hours. The reaction was then concentrated in vacuo and the crude product was purified via preparative HPLC. Fractions containing the desired product were concentrated and lyophilized to yield corresponding NAD⁺3.

NAD⁺3. A colorless solid, 45% yield; ¹H NMR (400 MHz, D₂O): δ 1.57-1.66 (m, 2H, CH₂), 1.95-2.12 (m, 2H, CH₂), 2.29 (t, 1H, J=2.4 Hz, CH), 3.63-3.76 (m, 2H, CH₂), 4.13-4.16 (m, 1H, CH₂), 4.24-4.39 (m, 3H, CH₂+CH₂), 4.40 (d, 1H, J=2.0 Hz, CH), 4.52 (t, 1H, J=4.4, CH), 4.60 (dd, 1H, J=4.4, 1.6 Hz, CH), 4.71 (t, 1H, J=5.6 Hz, CH), 4.76 (dd, 1H, J=5.6 Hz, CH), 4.92 (br, 1H, CH), 6.10 (d, 1H, J=6.6 Hz, CH), 6.63 (d, 1H, J=6.0 Hz, CH), 8.17 (dd, 1H, J=8.0, 6.0 Hz, ArH), 8.40 (s, 1H, ArH), 8.61 (s, 1H, ArH), 8.92 (d, 1H, J=8.0 Hz, ArH), 9.08 (d, 1H, J=6.0 Hz, ArH), 9.25 (s, 1H, ArH); HRMS (ESI) Calcd. For C₂₆H₃₄N₂O₁₄P₂⁺ (M+H)⁺ requires 730.1633, Found: 730.1639.

### Example 3. Synthesis of NAD⁺7-9

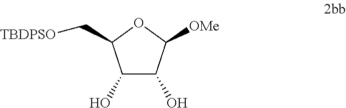

General procedure for the synthesis of (2R,3 S,4R,5R)-2-(((tert-butyldiphenylsilyl)oxy)methyl)-5-methoxytetrahydrofuran-3,4-diol (2bb): To a stirred solution of Methyl β-D-ribofuranoside (SM1) (1.0 eq) in DMF (24 mL) was added the Imidazole (2 eq) and TBDPSCl (1.1 eq) at 0° C. The reaction mixture was allowed to warm to room temperature. After stirring at this temperature for 24 hours, the reaction mixture was diluted with EtOAc (100 mL), and the organic phase was washed with water (5×50 mL), dried over anhydrous Na₂SO₄, filtered and concentrated to give a residue. The residue was purified by a flash column chromatography on silica gel to afford the compound 2bb.

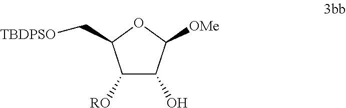

General procedure for the synthesis of compound 3bb: To a stirred solution of compound 2bb (1 eq) in anhydrous THF (25 mL) was added NaH (1.1 eq, 60% dispersion in mineral oil) at 0° C. followed by the addition of corresponding RBr or ROTf (1.5 eq) (R may be —(CH₂)ₙC≡CH wherein n is 1, 2, or 3) at the same temperature. Then reaction mixture was allowed to warm to room temperature. After stirring at this temperature for 6 hours, the reaction mixture was quenched with saturated aqueous NH₄Cl (20 mL) and extracted with EtOAc (3×50 mL). The combined organic layers were washed water (3×50 mL), dried over anhydrous Na₂SO₄, filtered and concentrated and purified by a flash column chromatography on silica gel to afford the corresponding compound 3bb.

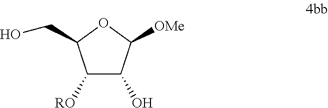

General procedure for the synthesis of compound 4bb: To a 0° C. solution of compound 3bb (1.0 eq) in anhydrous THF (25 mL) was added AcOH (1.5 eq) followed by the addition of TBAF (1.5 eq). Then reaction mixture was allowed to warm to room temperature. After stirring at this temperature for 14 hours, the reaction mixture was concentrated under reduced pressure to give a residue. The residue was purified by a flash column chromatography on silica gel to afford the corresponding compound 4bb.

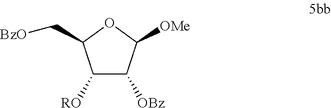

General procedure for the synthesis of compound 5bb: To a solution of compound 4bb (1.0 eq) in a mixture of anhydrous DCM (10 mL) and anhydrous pyridine (10 mL) was added BzCl (3 eq) at 0° C. Then the reaction mixture was allowed to warm to room temperature. After stirring for 24 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the corresponding compound 5bb.

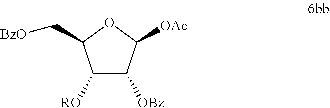

General procedure for the synthesis of compound 6bb: Compound 5bb (1.0 eq) was dissolved in a mixture of TFA/H₂O (9/1, 15 mL) and the resulting mixture was stirred at room temperature until the reaction complete (monitoring by TLC). Then the reaction was diluted with DCM (60 mL) and the solution was added dropwise to a stirred mixture of ice and saturated aqueous NaHCO₃. Solid NaHCO₃ was added during the addition to maintain a PH of 7. The mixture was extracted with DCM (3×50 mL), and the combined organic extracts was washed with H₂O (50 mL), brine (50 mL), dried over anhydrous Na₂SO₄, filtered, concentrated to give a residue. The residue was dissolved in pyridine (15 mL) and cooled to 0° C. Ac₂O (0.5 mL) was added dropwise and then the resulting mixture was allowed to warm to room temperature. After stirring for 6 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the corresponding compound 6bb.

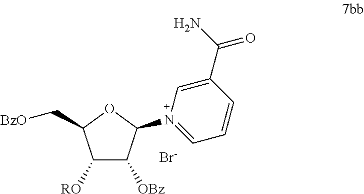

General procedure for the synthesis of compound 7bb: Compound 6bb (1.0 eq) was dissolved in toluene (10 mL) and cooled to 0° C. HBr (33 wt % in acetic acid) (1.5 eq) was added dropwise and the reaction was stirred at 0° C. for 5 hours. After the starting material was consumed, the reaction was concentrated under reduced pressure to give a residue. The residue was azeotroped with toluene (3×20 mL) to remove remaining acetic acid and dried in vacuo. The crude product and nicotinamide (103 mg, 0.84 mmol, 1.2 eq) was dissolved in CH₃CN (20 mL). The reaction was stirred under Ar gas at room temperature for 24 hours. The reaction was concentrated in vacuo (the temperature was kept below 35° C.) and purified by a flash column chromatography on silica gel to afford the corresponding compound 7bb.

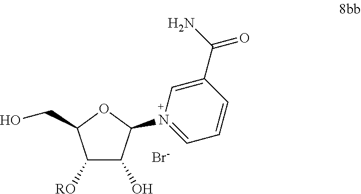

General procedure for the synthesis of compound 8bb: Compound 7bb (0.45 mmol) was dissolved in ammonia (18 mL, 7 N in MeOH) and the reaction was stirred at −10° C. for 48 hours. The reaction was concentrated under reduced pressure and the crude product was dissolved in MeOH (0.5 mL). Addition of ethyl ether (10 mL) resulted in ppt of the desired product. The procedure was repeated three times to yield the desired product 8bb.

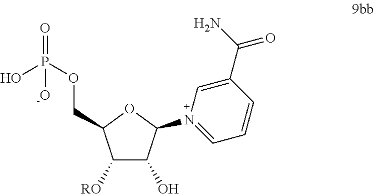

General procedure for the synthesis of compound 9bb: To a stirred solution of compound 8bb (0.27 mmol) in trimethylphosphate (2 mL) was added P(O)Cl₃ (175 μL, 1.89 mmol, 7 eq) at 0° C. and the resulting mixture was stirred at 0° C. for 6 hours. A few drops H₂O was then added to quench the reaction. Trimethylphosphate was removed by extraction with ethyl ether (3×20 ml). The remaining trimethylphosphate was removed by a second extraction with THF (5 ml). The aqueous layer was concentrated in vacuo and the crude product was dissolved in MeOH (0.5 mL). Addition of ethyl ether (10 mL) resulted in ppt of the desired product. The procedure was repeated three times to yield the desired product 9bb.

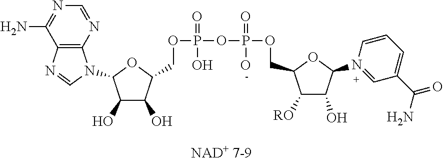

General procedure for the synthesis of NAD⁺7 and NAD⁺9: To a stirred solution of Adenosine 5′-monophosphate (5′-AMP) (52 mg, 0.15 mmol, 1.5 eq) in dried DMF (2 mL) were added 1,1-carbonyldiimidazole (CDI) (63 mg, 0.50 mmol, 5 eq) and triethylamine (23 μL, 0.16 mmol. 1.6 eq). The reaction mixture was stirred at room temperature for 14 hours, and then quenched with 0.100 ml dried methanol. The solvent was removed under vacuum and the residue was coevaporated 3 times each with 1.00 ml of dried DMF. The activated 5′-AMP was dissolved in dried DMF (1 mL) and compound 9bb (0.10 mmol, 1.0 eq) was added. After stirring at room temperature for 4 days, H₂O was added to quench the reaction at 0° C. The resulting mixture was continued stirring at room temperature for 24 hours. The reaction was then concentrated in vacuo and the crude product was purified via preparative HPLC. Fractions containing the desired product were concentrated and lyophilized to yield the corresponding NAD⁺7 and NAD⁺9.

The following Schemes 3 and 4 and reaction procedures provide additional conditions by which NAD⁺7 and NAD⁺9 may be prepared.

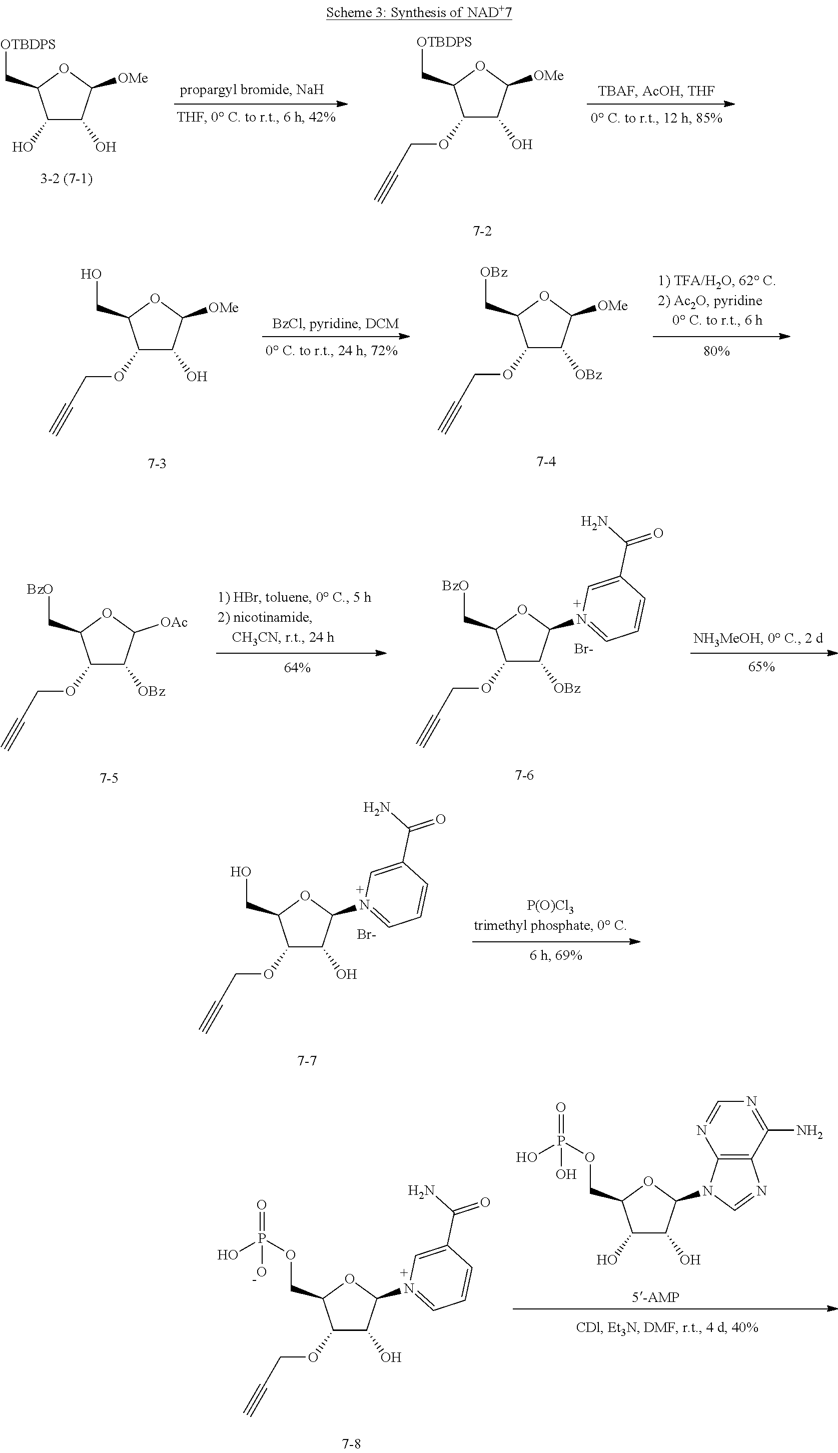

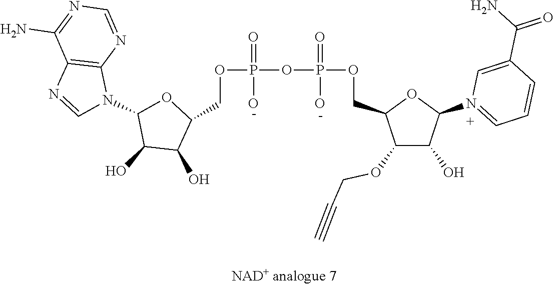

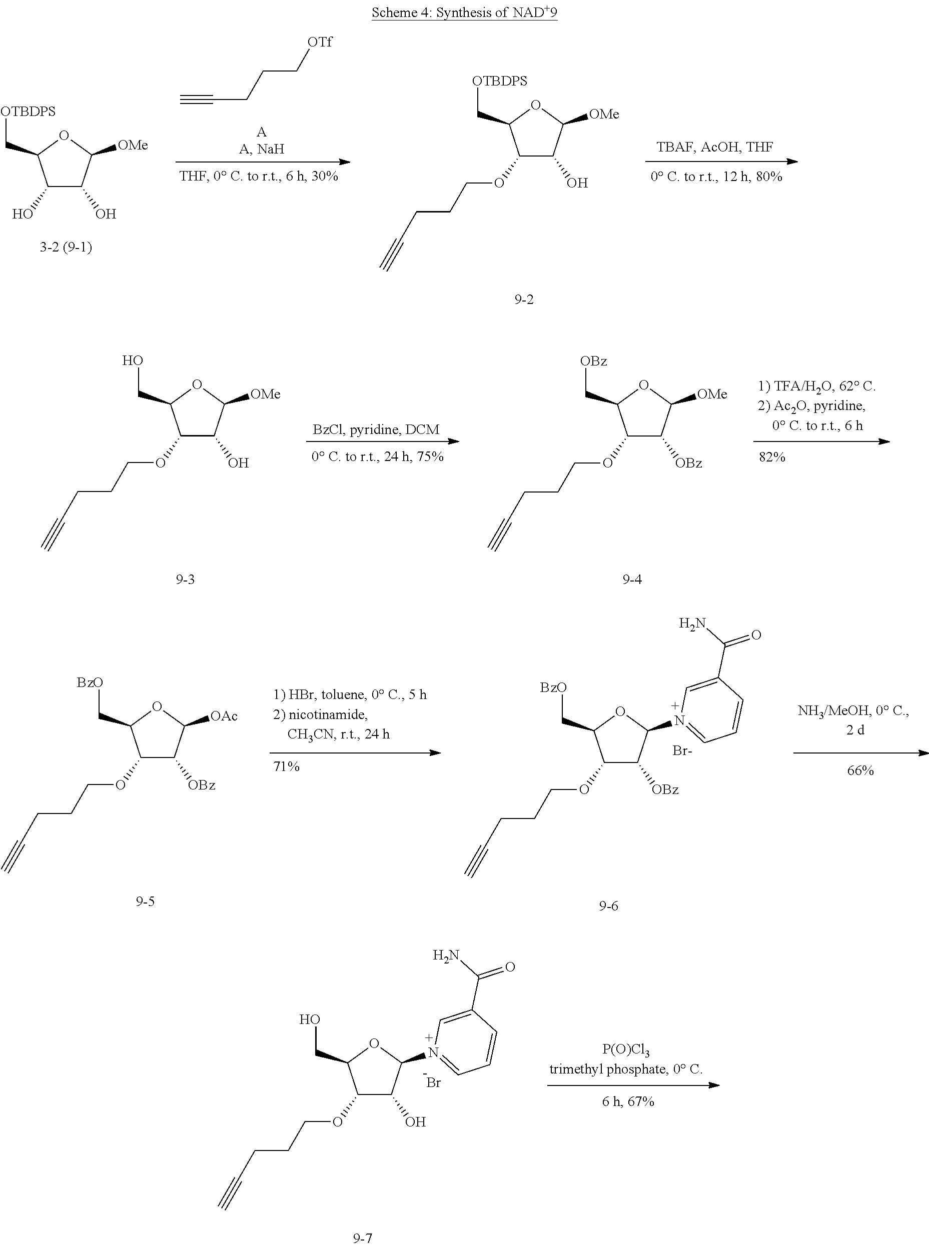

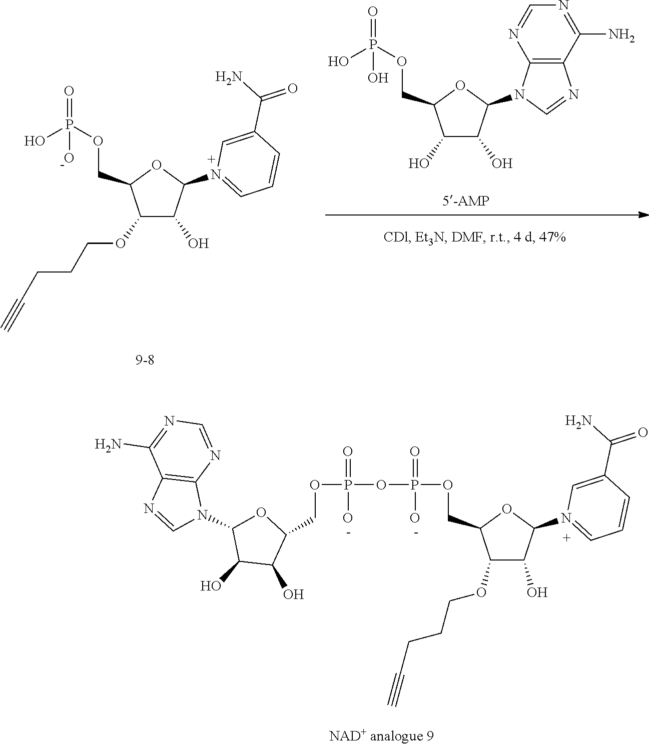

General procedure for the synthesis of compound 7-2 and 9-2: To a stirred solution of compound 3-2 (3.62 g, 9.0 mmol) in anhydrous THF (30 mL) was added NaH (432 mg, 10.8 mmol, 1.2 eq, 60% dispersion in mineral oil) at 0° C. followed by the addition of propargyl bromide (1.61 g, 13.5 mmol, 1.5 eq) or pent-4-yn-1-yl trifluoromethanesulfonate (2.92 g, 13.5 mmol, 1.5 eq) at the same temperature. Then the reaction mixture was allowed to warm to room temperature. After stirring at this temperature for 6-8 hours, the reaction mixture was quenched with saturated aqueous NH₄Cl (20 mL) and extracted with EtOAc (3×50 mL). The combined organic layers were washed water (3×50 mL), dried over anhydrous Na₂SO₄, filtered and concentrated and purified by a flash column chromatography on silica gel to afford the corresponding product 7-2 and 7-2.

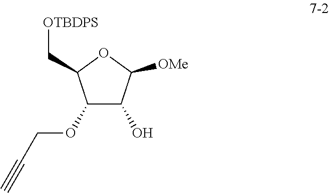

(2R,3R,4S,5R)-5-(((tert-butyldiphenylsilyl)oxy)methyl)-2-methoxy-4-(prop-2-yn-1-yloxy)tet-rahydrofuran-3-ol (7-2). A colorless oil, 1.67 g, 42% yield; ¹H NMR (400 MHz, CDCl₃): δ 1.07 (s, 9H, 3CH₃), 2.45 (t, 1H, J=2.4 Hz, CH), 2.59 (d, 1H, J=3.2 Hz, OH), 3.31 (s, 3H, OCH₃), 3.75 (dd, 1H, J=10.8, 4.4 Hz, CH₂), 3.80 (dd, 1H, J=10.8, 5.2 Hz, CH₂), 4.09-4.13 (m, 1H, CH), 4.14-4.19 (m, 2H, CH+CH₂), 4.24-4.29 (m, 2H, CH+CH₂), 4.87 (s, 1H, CH), 7.36-7.45 (m, 6H, ArH), 7.68-7.71 (m, 4H, ArH); ¹³C NMR (100 MHz, CDCl₃): δ 19.4, 26.9, 55.3, 58.3, 64.6, 73.8, 75.6, 79.3, 79.7, 81.8, 108.4, 127.85, 127.87, 129.87, 129.91, 133.4, 135.76, 135.79.

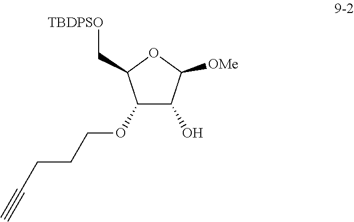

(2R,3R,4S,5R)-5-(((tert-butyldiphenylsilyl)oxy)methyl)-2-methoxy-4-(pent-4-yn-1-yloxy)tet-rahydrofuran-3-ol (9-2). A colorless solid, 1.27 g, 30% yield; ¹H NMR (400 MHz, CD₃Cl): δ 1.07 (s, 9H, 3CH₃), 1.75-1.83 (m, 2H, CH₂), 1.95 (t, 2H, J=2.4 Hz, CH), 2.21-2.35 (m, 2H, CH₂), 3.31 (s, 3H, OCH₃), 3.57-3.65 (m, 2H, CH₂), 3.70-3.80 (m, 2H, CH₂), 4.07-4.09 (m, 3H, 3CH), 4.86 (s, 1H, CH), 7.36-7.45 (m, 6H, ArH), 7.68-7.70 (m, 4H, ArH); ¹³C NMR (100 MHz, CD₃Cl): δ 15.1, 19.3, 26.8, 28.1, 55.1, 64.6, 68.9, 69.1, 73.5, 79.5, 81.9, 83.3, 108.3, 127.68, 127.70, 129.70, 129.74, 133.3, 135.59, 135.61.

General procedure for the synthesis of compound 7-3 and 9-3: To a 0° C. solution of compound 7-2 (1.10 g, 2.5 mmol) or 9-2 (1.17 g, 2.5 mmol) in anhydrous THF (25 mL) was added AcOH (225 mg, 3.75 mmol, 1.5 eq) followed by the addition of TBAF (3.75 mL, 3.75 mmol, 1.0 M in THF, 1.5 eq). Then the reaction mixture was allowed to warm to room temperature. After stirring at this temperature for 14 hours, the reaction mixture was concentrated under reduced pressure to give a residue. The residue was purified by a flash column chromatography on silica gel to afford the corresponding compound 7-3 and 9-3.

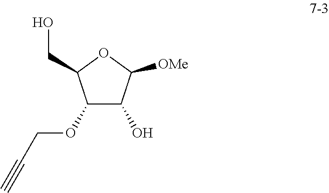

(2R,3R,4S,5R)-5-(hydroxymethyl)-2-methoxy-4-(prop-2-yn-1-yloxy)tetrahydrof-uran-3-ol (7-3). A colorless oil, 430 mg, 85% yield; ¹H NMR (400 MHz, CDCl₃): δ 2.53 (t, 1H, J=2.4 Hz, CH), 3.40 (s, 3H, OCH₃), 3.63 (dd, 1H, J=12.0, 4.0 Hz, CH₂), 3.81 (dd, 1H, J=12.0 3.2 Hz, CH₂), 4.14-4.19 (m, 2H, 2CH), 4.19-4.30 (m, 3H, CH+CH₂), 4.87 (s, 1H, CH); ¹³C NMR (100 MHz, CDCl₃): δ 55.7, 58.4, 62.7, 73.6, 75.7, 78.4, 79.0, 82.3, 108.8.

(2R,3R,4S,5R)-5-(hydroxymethyl)-2-methoxy-4-(pent-4-yn-1-yloxy)tetrahydrofuran-3-ol (9-3). A colorless oil, 461 mg, 80% yield; ¹H NMR (400 MHz, CD₃Cl): δ 1.74-1.86 (m, 2H, CH₂), 1.98 (t, 2H, J=2.8 Hz, CH), 2.27-2.34 (m, 2H, CH₂), 3.40 (s, 3H, OCH₃), 3.58-3.70 (m, 3H, CH₂+CH₂), 3.81 (dd, 1H, J=11.6, 2.4 Hz, CH₂), 4.07-4.14 (m, 3H, 3CH), 4.86 (s, 1H, CH); ¹³C NMR (100 MHz, CD₃Cl): δ 15.0, 28.0, 55.7, 63.0, 69.1, 69.2, 73.5, 78.4, 82.5, 83.1, 108.9.

General procedure for the synthesis of compound 7-4 and 9-4: To a solution of 7-3 (404 mg, 2.0 mmol) or 9-3 (460 mg, 2.0 mmol) in a mixture of anhydrous DCM (10 mL) and anhydrous pyridine (10 mL) was added BzCl (691 μL, 6.0 mmol, 3 eq) at 0° C. Then the reaction mixture was allowed to warm to room temperature. After stirring for 24 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the corresponding compound 7-4 and 9-4.

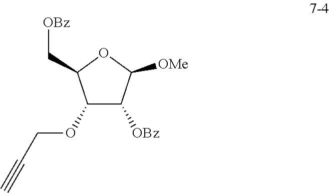

((2R,3R,4R,5R)-4-(benzoyloxy)-5-methoxy-3-(prop-2-yn-1-yloxy)tetrahydrofuran-2-yl)met-hyl benzoate (7-4). A colorless oil, 591 mg, 72% yield; ¹H NMR (400 MHz, CDCl₃): δ 2.35 (t, 1H, J=2.4 Hz, CH), 3.36 (s, 3H, OCH₃), 4.22-4.23 (m, 2H, CH₂), 4.42-4.48 (m, 2H, CH+CH₂), 4.62-4.69 (m, 2H, CH+CH₂), 5.05 (s, 1H, CH), 5.49 (d, 1H, J=4.4 Hz, CH), 7.44-7.48 (m, 4H, ArH), 7.56-7.61 (m, 2H, ArH), 8.07-8.13 (m, 4H, ArH); ¹³C NMR (100 MHz, CDCl₃): δ 55.1, 58.3, 64.2, 73.9, 75.3, 76.8, 128.3, 128.4, 129.4, 129.7, 129.87, 129.93, 133.1, 133.4, 165.6, 166.39.

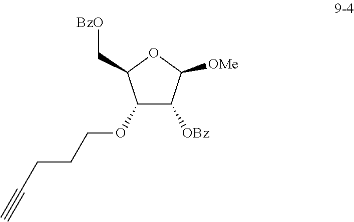

((2R,3R,4R,5R)-4-(benzoyloxy)-5-methoxy-3-(pent-4-yn-1-yloxy)tetrahydrofuran-2-yl)met-hyl benzoate (9-4). A colorless solid, 658 mg, 75% yield; ¹H NMR (400 MHz, CD₃Cl): δ 1.63-1.70 (m, 2H, CH₂), 1.82 (t, 2H, J=2.4 Hz, CH), 2.12-2.17 (m, 2H, CH₂), 3.36 (s, 3H, OCH₃), 3.54-3.59 (m, 1H, CH₂), 3.65-3.71 (m, 1H, CH₂), 4.30 (dd, 1H, J=7.2, 4.0 Hz, CH), 4.43 (m, 2H, CH₂+CH), 4.65 (dd, 1H, J=13.2, 5.2 Hz, CH₂), 5.02 (s, 1H, CH), 5.49 (d, 1H, J=4.0 Hz, CH), 7.44-7.48 (m, 4H, ArH), 7.56-7.61 (m, 2H, ArH), 8.08 (d, 2H, J=7.6 Hz, ArH), 8.12 (d, 2H, J=7.2 Hz, ArH); ¹³C NMR (100 MHz, CD₃Cl): δ 15.0, 28.5, 55.2, 64.7, 68.6, 69.6, 74.1, 78.5, 79.1, 83.4, 106.3, 128.4, 128.5, 129.5, 129.7, 129.8, 129.9, 133.1, 133.4, 165.5, 166.4.

General procedure for the synthesis of compound 7-5 and 9-5: Compound 7-4 (533 mg, 1.3 mmol) or 9-4 (570 mg, 1.3 mmol) was dissolved in a mixture of TFA/H₂O (9/1, 15 mL) and the resulting mixture was stirred at room temperature until the reaction complete (monitoring by TLC). Then the reaction was diluted with DCM (60 mL) and the solution was added dropwise to a stirred mixture of ice and saturated aqueous NaHCO₃. Solid NaHCO₃ was added during the addition to maintain a PH of 7. The mixture was extracted with DCM (3×50 mL), and the combined organic extracts was washed with H₂O (50 mL), brine (50 mL), dried over anhydrous Na₂SO₄, filtered, concentrated to give a residue. The residue was dissolved in pyridine (15 mL) and cooled to 0° C. Ac₂O (0.5 mL) was added dropwise and then the resulting mixture was allowed to warm to room temperature. After stirring for 6 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the corresponding compound 7-5 and 9-5.

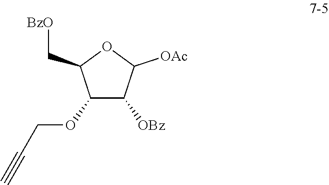

((2R,3R,4R)-5-acetoxy-4-(benzoyloxy)-3-(prop-2-yn-1-yloxy)tetrahy drofuran-2-yl)methyl benzoate (7-5). A colorless oil, 456 mg, 80% yield; ¹H NMR (400 MHz, CDCl₃): δ 1.95 (s, 3H, CH₃), 2.38 (t, 1H, J=2.4 Hz, CH), 4.25 (d, 2H, J=2.4 Hz, CH₂), 4.43-4.49 (m, 2H, CH₂), 4.66 (dd, 1H, J=8.0, 4.4 Hz, CH), 4.72-4.76 (m, 1H, CH), 5.56 (d, 1H, J=4.4 Hz, CH), 6.32 (s, 1H, CH), 7.43-7.48 (m, 4H, ArH), 7.56-7.60 (m, 2H, ArH), 8.06-8.12 (m, 4H, ArH); ¹³C NMR (100 MHz, CDCl₃): δ 20.9, 58.5, 63.1, 73.6, 75.6, 75.7, 78.8, 79.9, 98.5, 128.4, 128.5, 129.1, 129.7, 129.85, 129.93, 133.2, 133.6, 165.4, 166.0, 169.0.

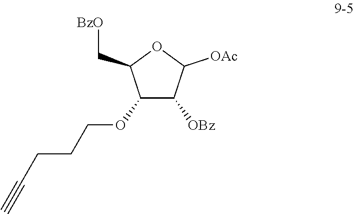

((2R,3R,4R)-5-acetoxy-4-(benzoyloxy)-3-(pent-4-yn-1-yloxy)tetrahydrofuran-2-yl)methyl benzoate (9-5). A colorless oil, 497 mg, 82% yield; ¹H NMR (400 MHz, CD₃Cl): δ 1.63-1.70 (m, 2H, CH₂), 1.83 (t, 2H, J=2.8 Hz, CH), 1.96 (s, 3H, CH₃), 2.12-2.17 (m, 2H, CH₂), 3.60 (dt, 1H, J=9.2, 6.0 Hz, CH₂), 3.72 (dt, 1H, J=9.2, 5.5 Hz, CH₂), 4.30 (dd, 1H, J=8.4, 4.4 Hz, CH), 4.42-4.48 (m, 2H, CH₂+CH), 4.70 (dt, 1H, J=13.2, 2.4 Hz, CH₂), 5.57 (d, 1H, J=4.4 Hz, CH), 6.31 (s, 1H, CH), 7.43-7.48 (m, 4H, ArH), 7.56-7.62 (m, 2H, ArH), 8.06-8.12 (m, 4H, ArH); ¹³C NMR (100 MHz, CD₃Cl): δ 14.9, 20.9, 28.4, 63.7, 68.6, 69.8, 73.7, 77.6, 78.0, 83.3, 98.4, 128.4, 128.5, 129.1, 129.68, 129.73, 129.8, 133.2, 133.5, 165.2, 166.1, 168.9.

General procedure for the synthesis of 7-6 and 9-6: Compound 9-5 (307 mg, 0.70 mmol) or 9-5 (327 mg, 0.70 mmol) was dissolved in toluene (10 mL) and cooled to 0° C. HBr (33 wt % in acetic acid) (257 mg, 1.05 mmol, 1.5 eq) was added dropwise and the reaction was stirred at 0° C. for 5 hours. After the starting material was consumed, the reaction was concentrated under reduced pressure to give a residue. The residue was azeotroped with toluene (3×20 mL) to remove remaining acetic acid and dried in vacuo. The crude product and nicotinamide (103 mg, 0.84 mmol, 1.2 eq) was dissolved in CH₃CN (20 mL). The reaction was stirred under Ar gas at room temperature for 24 hours. The reaction was concentrated in vacuo (the temperature was kept below 35° C.) and purified by a flash column chromatography on silica gel to afford the corresponding compound 7-6 and 9-6.

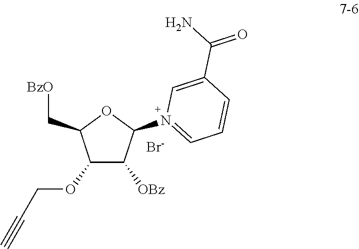

1-((2R,3R,4R,5R)-3-(benzoyloxy)-5-((benzoyloxy)methyl)-4-(prop-2-yn-1-yloxy)tetrahydrof-uran-2-yl)-3-carbamoylpyridin-1-ium bromide (7-6). A colorless solid, 260 mg, 64% yield; ¹H NMR (400 MHz, CDCl₃): δ 2.34 (t, 1H, J=2.8 Hz, CH), 4.35 (dd, 1H, J=16.0, 2.0 Hz, CH₂), 4.43 (dd, 1H, J=16.0, 2.0 Hz, CH₂), 4.80-4.84 (m, 1H, CH), 4.91 (d, 2H, J=2.8 Hz, CH₂), 5.13 (dd, 1H, J=8.4, 4.8 Hz, CH), 6.33 (d, 1H, J=5.6 Hz, CH), 6.50 (br, 1H, NH), 6.70 (s, 1H, CH), 7.44-7.52 (m, 4H, ArH), 7.59-7.63 (m, 2H, ArH), 7.93-7.97 (m, 1H, ArH), 8.07-8.13 (m, 4H, ArH), 9.08 (d, 1H, J=7.2 Hz, ArH), 9.16 (br, 1H, NH), 9.25 (d, 1H, J=6.0 Hz, ArH), 10.07 (s, 1H, ArH); ¹³C NMR (100 MHz, CDCl₃): δ 59.2, 62.7, 74.4, 75.8, 76.3, 79.2, 82.5, 98.5, 128.4, 128.6, 128.8, 128.9, 129.3, 130.0, 130.3, 133.9, 134.2, 134.6, 141.5, 142.0, 146.8, 163.6, 166.3, 171.3.

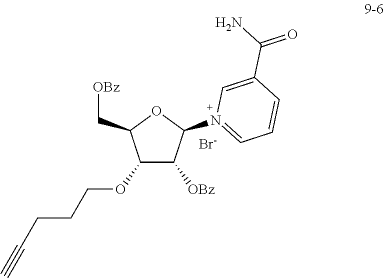

1-((2R,3R,4R,5R)-3-(benzoyloxy)-5-((benzoyloxy)methyl)-4-(pent-4-yn-1-yloxy)tetrahydro-furan-2-yl)-3-carbamoylpyridin-1-ium bromide (9-6). A colorless solid, 303 mg, 71% yield; ¹H NMR (400 MHz, CD₃OD): δ 1.67-1.74 (m, 2H, CH₂), 2.08 (t, 1H, J=2.4 Hz, CH), 2.19 (td, 2H, J=6.8, 2.4 Hz, CH₂), 3.70 (t, 2H, J=6.0 Hz, CH₂), 4.54-4.55 (m, 1H, CH), 4.87-4.95 (m, 3H, CH₂+CH), 5.91-5.94 (m, 1H, CH), 6.76 (s, 1H, CH), 7.51 (t, 2H, J=8.0 Hz, ArH), 7.57 (t, 2H, J=8.0 Hz, ArH), 7.63-7.67 (m, 1H, ArH), 7.69-7.73 (m, 1H, ArH), 8.06-8.08 (m, 2H, ArH), 8.18-8.20 (m, 2H, ArH), 8.24 (dd, 1H, J=8.0, 6.4 Hz, ArH), 9.04 (d, 1H, J=8.0 HZ, ArH), 9.40 (d, 1H, J=6.4 HZ, ArH), 9.66 (s, 1H, ArH); ¹³C NMR (100 MHz, CD₃OD): δ 15.6, 29.7, 64.1, 70.0, 70.7, 76.8, 78.2, 84.0, 84.5, 99.7, 129.9, 130.65, 130.68, 130.7, 131.1, 134.8, 135.3, 136.0, 142.1, 143.8, 146.8, 164.9, 167.3, 167.5.

General procedure for the synthesis of 7-7 and 9-7: Compound 9-6 (233 mg, 0.40 mmol) or 9-6 (244 mg, 0.40 mmol) was dissolved in ammonia (20 mL, 7 N in MeOH) and the reaction was stirred at 0° C. for 48 hours. The reaction was concentrated under reduced pressure and the crude product was dissolved in MeOH (0.5 mL). Addition of ethyl ether (10 mL) resulted in ppt of the desired product. The procedure was repeated three times to yield the corresponding desired product 7-7 and 9-7.

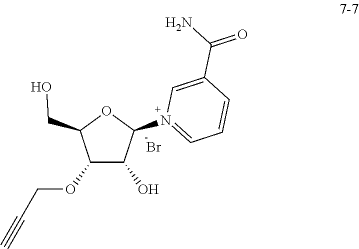

3-carbamoyl-1-((2R,3R,4S,5R)-3-hydroxy-5-(hydroxymethyl)-4-(prop-2-yn-1-yloxy)tetrahy-drofuran-2-yl)pyridin-1-ium bromide (7-7). A colorless solid, 97 mg, 65% yield; ¹H NMR (400 MHz, D₂O): δ 2.91 (t, 1H, J=2.4 Hz, CH), 4.15 (ddd, 1H, J=12.0, 5.2, 2.0 Hz, CH₂), 4.31 (ddd, 1H, J=12.0, 4.4, 2.4 Hz, CH₂), 4.10-4.34 (m, 3H, CH+CH₂), 4.66 (t, 1H, J=5.2 Hz, CH), 6.19 (d, 1H, J=5.6 Hz, CH), 8.28 (dd, 1H, J=8.0, 6.0 Hz, ArH), 8.97 (dt, 1H, J=8.0, 1.6 Hz, ArH), 9.26 (d, 1H, J=6.0 Hz, ArH), 9.44 (s, 1H, ArH); ¹³C NMR (100 MHz, D₂O): δ 57.96, 57.99, 60.0, 76.1, 76.4, 76.5, 85.8, 99.9, 128.3, 133.9, 140.3, 142.6, 145.7, 165.7.

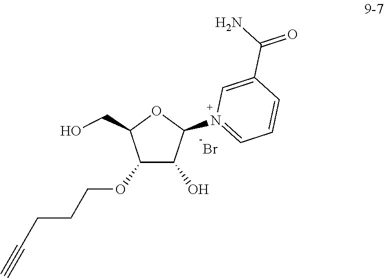

3-carbamoyl-1-((2R,3R,4R,5R)-4-hydroxy-5-(hydroxymethyl)-3-(pent-4-yn-1-yloxy)tetrah-ydrofuran-2-yl)pyridin-1-ium bromide (9-7). A colorless solid, 106 mg, 66% yield; ¹H NMR (400 MHz, D₂O): δ 1.79-1.86 (m, 2H, CH₂), 2.32-2.36 (m, 3H, CH+CH₂), 3.72-3.81 (m, 2H, CH₂), 3.91 (dd, 1H, J=12.8, 3.6 Hz, CH₂), 4.08 (dd, 1H, J=12.8, 2.8 Hz, CH₂), 4.16 (t, 1H, J=4.8 Hz, CH), 4.55-4.58 (m, 1H, CH), 4.66 (t, 1H, J=4.8 Hz, CH), 6.28 (d, 1H, J=4.8 Hz, CH), 8.29 (dd, 1H, J=8.4, 6.8 Hz, ArH), 8.99 (d, 1H, J=8.4 Hz, ArH), 9.27 (d, 1H, J=6.8 Hz, ArH), 9.62 (s, 1H, ArH); ¹³C NMR (100 MHz, D₂O): δ 14.3, 27.4, 60.1, 69.3, 69.5, 76.1, 76.9, 84.8, 85.7, 100.1, 128.3, 133.9, 140.3, 142.6, 145.6, 165.7.

General procedure for the synthesis of 7-8 and 9-8: To a stirred solution of compound 7-7 (82 mg, 0.22 mmol) or 9-7 (88 mg, 0.22 mmol) in trimethylphosphate (2 mL) was added P(O)Cl₃ (143 μL, 1.54 mmol, 7 eq) at 0° C. and the resulting mixture was stirred at 0° C. for 6 hours. A few drops H₂O was then added to quench the reaction. Trimethylphosphate was removed by extraction with ethyl ether (3×20 ml). The remaining trimethylphosphate was removed by a second extraction with THF (5 ml). The aqueous layer was concentrated in vacuo and the crude product was dissolved in MeOH (0.5 mL). Addition of ethyl ether (10 mL) resulted in ppt of the desired product. The procedure was repeated three times to yield the corresponding desired product 7-8 and 9-8.

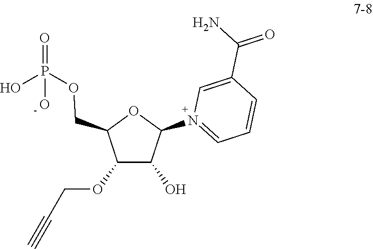

((2R,3S,4R,5R)-5-(3-carbamoylpyridin-1-ium-1-yl)-4-hydroxy-3-(prop-2-yn-1-yloxy)tetrah-ydrofuran-2-yl)methyl hydrogen phosphate (7-8). A colorless solid, 57 mg, 69% yield; ¹H NMR (400 MHz, D₂O): δ 1.52 (t, 1H, J=2.8 Hz, CH), 3.90 (dd, 1H, J=12.8, 2.8 Hz, CH₂), 4.06 (dd, 1H, J=12.8, 2.8 Hz, CH₂), 4.33-4.39 (m, 3H, CH+CH₂), 4.59-4.61 (m, 1H, CH), 4.67 (t, 1H, J=4.4 Hz, CH), 6.25 (d, 1H, J=4.0 Hz, CH), 8.25-8.29 (m, 1H, ArH), 8.97 (d, 1H, J=8.4 Hz, ArH), 9.26 (d, 1H, J=6.0 Hz, ArH), 9.59 (s, 1H, ArH); ¹³C NMR (100 MHz, D₂O): δ 58.1, 64.2 (d, J=4.9 Hz), 76.5, 76.7, 77.8, 78.9, 85.2 (d, J=9.3 Hz), 99.8, 128.5, 133.9, 139.8, 142.4, 146.0, 165.7.

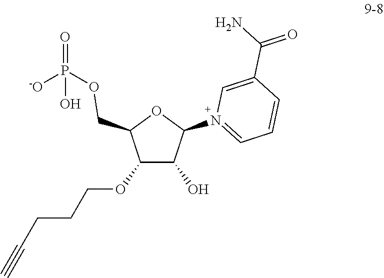

((2R,3R,4R,5R)-5-(3-carbamoylpyridin-1-ium-1-yl)-3-hydroxy-4-(pent-4-yn-1-yloxy)tetrah-ydrofuran-2-yl)methyl hydrogen phosphate (9-8). A colorless solid, 59 mg, 67% yield; ¹H NMR (400 MHz, D₂O): δ 1.79-1.86 (m, 2H, CH₂), 2.30-2.34 (m, 3H, CH+CH₂), 3.78 (t, 2H, J=6.4 Hz, CH₂), 4.12-4.17 (m, 1H, J=12.8, CH₂), 4.22 (dd, 1H, J=4.8, 2.4 Hz, CH), 4.30-4.35 (m, 1H, CH₂), 4.63 (t, 1H, J=4.8 Hz, CH), 4.73 (t, 1H, J=2.4 Hz, CH), 6.20 (d, 1H, J=4.8 Hz, CH), 8.28 (dd, 1H, J=8.0, 6.4 Hz, ArH), 8.97 (d, 1H, J=8.0 Hz, ArH), 9.25 (d, 1H, J=6.4 Hz, ArH), 9.43 (s, 1H, ArH); ¹³C NMR (100 MHz, D₂O, TMS): δ 14.3, 27.4, 64.4 (d, J=4.5 Hz), 69.3, 69.5, 76.8, 78.2, 85.3 (d, J=9.8 Hz), 100.0, 128.4, 133.9, 139.8, 142.4, 145.9, 165.7.

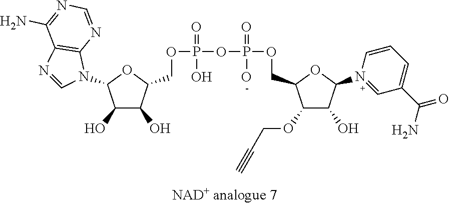

General procedure for the synthesis of NAD⁺ analogue 7 and 9: To a stirred solution of Adenosine 5′-monophosphate (5′-AMP) (52 mg, 0.15 mmol, 1.5 eq) in dried DMF (2 mL) were added 1,1-carbonyldiimidazole (CDI) (63 mg, 0.50 mmol, 5 eq) and triethylamine (23 μL, 0.16 mmol. 1.6 eq). The reaction mixture was stirred at room temperature for 14 hours, and then quenched with 0.100 ml dried methanol. The solvent was removed under vacuum and the residue was coevaporated 3 times each with 1.00 ml of dried DMF. The activated 5′-AMP was dissolved in dried DMF (1 mL) and compound (7-8) or 9-8 (0.10 mmol, 1.0 eq) was added. After stirring at room temperature for 4 days, H₂O was added to quench the reaction at 0° C. The resulting mixture was continued stirring at room temperature for 24 hours. The reaction was then concentrated in vacuo and the crude product was purified via preparative HPLC. Fractions containing the desired product were concentrated and lyophilized to yield the corresponding NAD⁺7 and NAD⁺9.

NAD⁺ analogue 7. A colorless solid, 40% yield; ¹H NMR (400 MHz, D₂O): δ 2.91 (t, 1H, J=2.0 Hz, CH), 4.25 (br, 3H, CH₂+CH₂), 4.35-4.44 (m, 5H, 2CH₂+CH), 4.51 (br, 1H, CH), 4.64 (t, 1H, J=5.2, CH), 4.73-4.77 (m, 2H, 2CH), 6.11-6.13 (m, 2H, 2CH), 8.24-8.28 (m, 1H, ArH), 8.32 (br, 1H, ArH), 8.65 (br, 1H, ArH), 8.90 (d, 1H, J=7.6 Hz, ArH), 9.22 (d, 1H, J=5.6 Hz, ArH), 9.39 (s, 1H, ArH); HRMS (ESI) Calcd. For C₂₄H₂₈N₇Na₂O₁₄P₂⁺ (M+2Na-2H)⁺ requires 746.0965, Found: 746.0958.

NAD⁺ analogue 9. A colorless solid, 47% yield; ¹H NMR (400 MHz, D₂O): δ 1.76-1.87 (m, 2H, CH₂), 2.29-2.34 (m, 2H, CH₂), 3.76 (t, 1H, J=2.4 Hz, CH), 4.18-4.28 (m, 4H, 2CH₂), 4.38-4.42 (m, 2H, CH₂), 4.51 (t, 1H, J=4.0 Hz, CH), 4.63 (t, 1H, J=5.6, CH), 4.71-4.74 (m, 2H, 2CH), 6.13 (d, 1H, J=5.2 Hz, CH), 6.16 (d, 1H, J=6.0 Hz, CH), 8.29 (dd, 1H, J=8.4, 6.0 Hz, ArH), 8.40 (s, 1H, ArH), 8.60 (s, 1H, ArH), 8.94 (d, 1H, J=8.4 Hz, ArH), 9.26 (d, 1H, J=6.0 Hz, ArH), 9.42 (s, 1H, ArH); HRMS (ESI) Calcd. For C₂₆H₃₄N₂O₁₄P₂⁺ (M+H)⁺ requires 730.1633, Found: 730.1631.

NAD⁺8 is prepared following the procedure as described above with the necessary modifications well-understood by the skilled artisan.

### Example 4. Synthesis of NAD⁺10-18

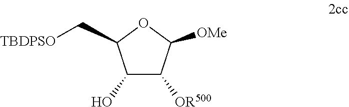

General procedure for the synthesis of compound 2cc: To a stirred solution of (2R,3 S,4R,5R)-2-(((tert-butyldiphenylsilyl)oxy)methyl)-5-methoxytetrahydrofuran-3,4-diol (1 eq) in anhydrous THF (25 mL) was added NaH (1.1 eq, 60% dispersion in mineral oil) at 0° C. followed by the addition of R⁵⁰⁰Br (1.5 eq) at the same temperature. If the target compound is, for example, NAD⁺14, R⁵⁰⁰ is propargyl. Other compounds NAD⁺10-13 and 15-18 may use the corresponding alkyl bromide. Then reaction mixture was allowed to warm to room temperature. After stirring at this temperature for 6 hours, the reaction mixture was quenched with saturated aqueous NH₄Cl (20 mL) and extracted with EtOAc (3×50 mL). The combined organic layers were washed water (3×50 mL), dried over anhydrous Na₂SO₄, filtered and concentrated and purified by a flash column chromatography on silica gel to afford the compound 2cc.

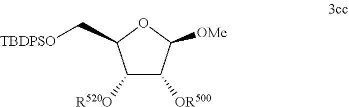

General procedure for the synthesis of compound 3cc: To a stirred solution of compound 2bb (1 eq) in anhydrous THF (25 mL) was added NaH (1.1 eq, 60% dispersion in mineral oil) at 0° C. followed by the addition of corresponding R⁵²⁰Br or R⁵²⁰OTf (1.5 eq) at the same temperature. If the target compound is, for example, NAD⁺14, R⁵²⁰ is —CH₂—Chx, wherein Chx is cyclohexyl. Other compounds NAD⁺10-13 and 15-18 may use the corresponding alkyl bromide or alkyl triflate. Then reaction mixture was allowed to warm to room temperature. After stirring at this temperature for 6 hours, the reaction mixture was quenched with saturated aqueous NH₄Cl (20 mL) and extracted with EtOAc (3×50 mL). The combined organic layers were washed water (3×50 mL), dried over anhydrous Na₂SO₄, filtered and concentrated and purified by a flash column chromatography on silica gel to afford the compound 3cc.

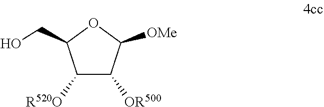

General procedure for the synthesis of compound 4cc: To a 0° C. solution of compound 3cc (2.1 mmol) in anhydrous THF (25 mL) was added AcOH (180 μL, 3.2 mmol, 1.5 eq) followed by the addition of TBAF (3.2 mL, 3.2 mmol, 1.0 M in THF, 1.5 eq). Then reaction mixture was allowed to warm to room temperature. After stirring at this temperature for 14 hours, the reaction mixture was concentrated under reduced pressure to give a residue. The residue was purified by a flash column chromatography on silica gel to afford the corresponding compound 4cc.

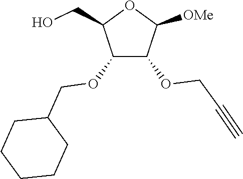 [SMILES: C#CCO[C@H]1[C@H](OC)O[C@H](CO)[C@H]1OCC1CCCCC1]

((2R,3R,4R,5R)-3-(cyclohexylmethoxy)-5-methoxy-4-(prop-2-yn-1-yloxy)tetrahydrofuran-2-yl)methanol (4cc). A colorless oil; ¹H NMR (400 MHz, CDCl₃): δ 0.87-0.97 (m, 2H, CH₂), 1.12-1.29 (m, 3H, CH₂), 1.57-1.79 (m, 6H, CH₂), 2.47 (t, 1H, J=2.4 Hz, CH), 3.24 (dd, 1H, J=8.8, 6.4 Hz, CH₂), 3.38-3.42 (m, 4H, OCH₃+CH₂), 3.60 (dd, 1H, J=12.0, 3.2 Hz, CH₂), 3.83 (dd, 1H, J=12.0, 3.2 Hz, CH₂), 4.03 (dd, 1H, J=6.8, 4.4 Hz, CH), 4.09 (d, 1H, J=5.2 Hz, CH), 4.13-4.17 (m, 1H, CH), 4.34-4.35 (m, 1H, CH), 4.92 (s, 1H, CH).

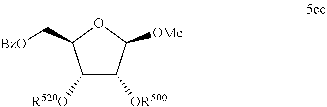

General procedure for the synthesis of compound 5cc: To a solution of compound 4cc (1.7 mmol) in a mixture of anhydrous DCM (10 mL) and anhydrous pyridine (10 mL) was added BzCl (588 μL, 5.1 mmol, 3 eq) at 0° C. Then the reaction mixture was allowed to warm to room temperature. After stirring for 24 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the corresponding compound 5cc.

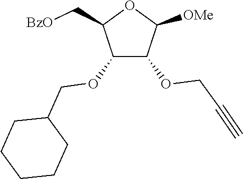

((2R,3R,4R,5R)-3-(cyclohexylmethoxy)-5-methoxy-4-(prop-2-yn-1-yloxy)tetrahydrofuran-2-yl)methyl benzoate (5cc). A colorless oil; ¹H NMR (400 MHz, CDCl₃): δ 0.84-0.96 (m, 2H, CH₂), 1.10-1.25 (m, 3H, CH₂), 1.57-1.77 (m, 6H, CH₂), 2.46 (t, 1H, J=2.4 Hz, CH), 3.23 (dd, 1H, J=8.8, 6.8 Hz, CH₂), 3.31 (s, 3H, OCH₃), 3.42 (dd, 1H, J=8.8, 6.0 Hz, CH₂), 4.07-4.10 (m, 1H, CH), 4.14 (d, 1H, J=4.4 Hz, CH), 44.31-4.42 (m, 4H, CH+2CH₂),4.54-4.59 (m, 1H, CH₂), 4.93 (s, 1H, CH), 7.44 (t, 2H, J=7.6 Hz, ArH), 7.56 (t, 1H, J=7.6 Hz, ArH), 8.08 (dd, 2H, J=7.6, 0.8 Hz, ArH).

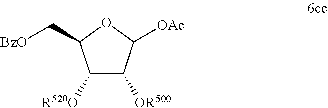

General procedure for the synthesis of compound 6cc: Compound 5cc (1.3 mmol) was dissolved in a mixture of TFA/H₂O (9/1, 15 mL) and the resulting mixture was stirred at room temperature until the reaction complete (monitoring by TLC). Then the reaction was diluted with DCM (60 mL) and the solution was added dropwise to a stirred mixture of ice and saturated aqueous NaHCO₃. Solid NaHCO₃ was added during the addition to maintain a PH of 7. The mixture was extracted with DCM (3×50 mL), and the combined organic extracts was washed with H₂O (50 mL), brine (50 mL), dried over anhydrous Na₂SO₄, filtered, concentrated to give a residue. The residue was dissolved in pyridine (15 mL) and cooled to 0° C. Ac₂O (0.5 mL) was added dropwise and then the resulting mixture was allowed to warm to room temperature. After stirring for 6 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the corresponding compound 6cc.

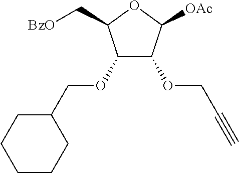

((2R,3R,4R)-5-acetoxy-3-(cyclohexylmethoxy)-4-(prop-2-yn-1-yloxy)tetrahydrofuran-2-yl)methyl benzoate (6cc). A colorless oil; ¹H NMR (400 MHz, CDCl₃): δ 0.86-0.98 (m, 2H, CH₂), 1.11-1.27 (m, 3H, CH₂), 1.57-1.78 (m, 6H, CH₂), 1.94 (s, 3H, CH₃), 2.47 (t, 1H, J=2.4 Hz, CH), 3.26 (dd, 1H, J=8.8, 6.4 Hz, CH₂), 3.46 (dd, 1H, J=8.8, 6.4 Hz, CH₂), 4.07 (dd, 1H, J=7.6, 4.4 Hz, CH), 4.21 (d, 1H, J=4.4 Hz, CH), 4.34-4.45 (m, 4H, CH+2CH₂),4.64 (dd, 1H, J=13.6, 4.8 Hz, CH₂), 6.21 (s, 1H, CH), 7.44 (t, 2H, J=7.6 Hz, ArH), 7.57 (t, 1H, J=7.6 Hz, ArH), 8.08 (d, 2H, J=7.6 Hz, ArH).

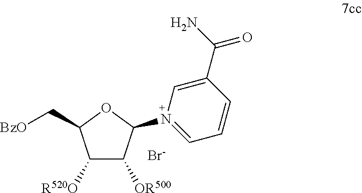

General procedure for the synthesis of compound 7cc: Compound 6cc (0.70 mmol) was dissolved in toluene (10 mL) and cooled to 0° C. HBr (33 wt % in acetic acid) (257 mg, 1.05 mmol, 1.5 eq) was added dropwise and the reaction was stirred at 0° C. for 5 hours. After the starting material was consumed, the reaction was concentrated under reduced pressure to give a residue. The residue was azeotroped with toluene (3×20 mL) to remove remaining acetic acid and dried in vacuo. The crude product and nicotinamide (103 mg, 0.84 mmol, 1.2 eq) was dissolved in CH₃CN (20 mL). The reaction was stirred under Ar gas at room temperature for 24 hours. The reaction was concentrated in vacuo (the temperature was kept below 35° C.) and purified by a flash column chromatography on silica gel to afford the corresponding compound 7cc.

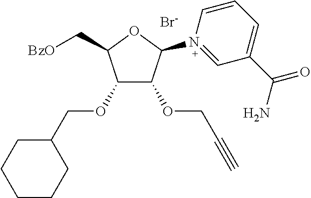

1-((2R,3R,4R,5R)-5-((benzoyloxy)methyl)-4-(cyclohexylmethoxy)-3-(prop-2-yn-1-yloxy)tetrahydrofuran-2-yl)-3-carbamoylpyridin-1-ium bromide. A colorless solid; ¹H NMR (400 MHz, CDCl₃): δ 0.69-0.82 (m, 2H, CH₂), 1.04-1.18 (m, 3H, CH₂), 1.30-1.66 (m, 6H, CH₂), 2.39 (t, 1H, J=2.8 Hz, CH), 3.28 (dd, 1H, J=8.8, 6.4 Hz, CH₂), 3.35 (dd, 1H, J=8.8, 6.4 Hz, CH₂), 4.16 (dd, 1H, J=4.0, 2.4 Hz, CH), 4.24 (dd, 1H, J=16.0, 2.4 Hz, CH₂), 4.51 (dd, 1H, J=16.0, 2.4 HZ, CH₂), 4.55 (d, 1H, J=2.8 Hz, CH₂), 5.0 (t, 1H, J=4.8 Hz, CH), 5.07 (dd, 1H, J=6.8, 3.6 Hz, CH), 6.21 (s, 1H, CH), 7.12 (d, 1H, J=5.6 Hz, CH), 7.52 (t, 2H, J=7.6 Hz, ArH), 7.62 (t, 1H, J=7.6 Hz, ArH), 7.98-8.02 (m, 1H, ArH), 8.08 (d, 2H, J=7.6 Hz, ArH), 8.99 (d, 1H, J=6.0 Hz, ArH), 9.14 (d, J=8.0 Hz, ArH), 9.22 (s, 1H, NH), 10.69 (s, 1H, ArH).

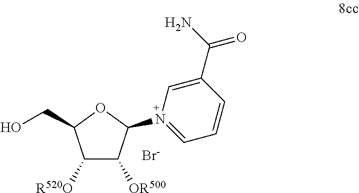

General procedure for the synthesis of compound 8cc: Compound 7cc (0.45 mmol) was dissolved in ammonia (18 mL, 7 N in MeOH) and the reaction was stirred at −10° C. for 48 hours. The reaction was concentrated under reduced pressure and the crude product was dissolved in MeOH (0.5 mL). Addition of ethyl ether (10 mL) resulted in ppt of the desired product. The procedure was repeated three times to yield the desired corresponding product 8cc.

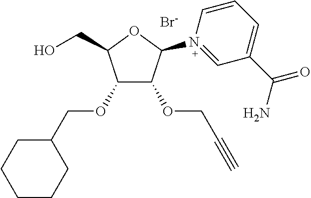

3-carbamoyl-1-((2R,3R,4R,5R)-4-(cyclohexylmethoxy)-5-(hydroxymethyl)-3-(prop-2-yn-1-yloxy)tetrahydrofuran-2-yl)pyridin-1-ium bromide. A colorless solid; ¹H NMR (400 MHz, D₂O): δ 0.54-0.64 (m, 2H, CH₂), 0.86-1.01 (m, 3H, CH₂), 1.12-1.29 (m, 3H, CH₂), 1.34-1.49 (m, 3H, CH₂), 2.97 (t, 1H, J=2.4 Hz, CH), 3.28 (dd, 1H, J=8.8, 5.2 Hz, CH₂), 3.33-3.37 (m, 1H, CH₂), 3.76 (dd, 1H, J=12.8, 4.4 Hz, CH₂), 3.87 (dd, 1H, J=12.8, 3.2 HZ, CH₂), 4.26 (d, 1H, J=4.8 Hz, CH), 4.31-4.41 (m, 2H, CH₂), 4.93 (t, 1H, J=5.6 Hz, CH), 5.0 (t, 1H, J=3.2 Hz, CH), 6.62 (d, 1H, J=5.6 Hz, CH), 8.17-8.21 (m, 1H, ArH), 8.96 (d, 1H, J=8.0 Hz, ArH), 9.04 (d, J=6.4 Hz, ArH), 9.28 (s, 1H, ArH).

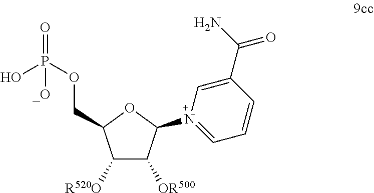

General procedure for the synthesis of compound 9cc: To a stirred solution of compound 8cc (0.27 mmol) in trimethylphosphate (2 mL) was added P(O)Cl₃ (175 μL, 1.89 mmol, 7 eq) at 0° C. and the resulting mixture was stirred at 0° C. for 6 hours. A few drops H₂O was then added to quench the reaction. Trimethylphosphate was removed by extraction with ethyl ether (3×20 ml). The remaining trimethylphosphate was removed by a second extraction with THF (5 ml). The aqueous layer was concentrated in vacuo and the crude product was dissolved in MeOH (0.5 mL). Addition of ethyl ether (10 mL) resulted in ppt of the desired product. The procedure was repeated three times to yield the desired corresponding product 9cc.

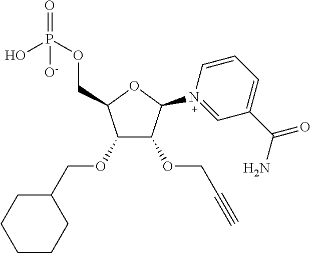

((2R,3R,4R,5R)-5-(3-carbamoylpyridin-1-ium-1-yl)-3-(cyclohexylmethoxy)-4-(prop-2-yn-1-yloxy)tetrahydrofuran-2-yl)methyl hydrogen phosphate. A colorless solid; ¹H NMR (400 MHz, D₂O): δ 0.69-0.80 (m, 2H, CH₂), 1.02-1.16 (m, 3H, CH₂), 1.27-1.44 (m, 3H, CH₂), 1.51-1.61 (m, 3H, CH₂), 2.94 (t, 1H, J=2.4 Hz, CH), 3.27 (dd, 1H, J=9.2, 5.2 Hz, CH₂), 3.31-3.37 (m, 1H, CH₂), 4.01-4.06 (m, 1H, CH₂), 4.11-4.16 (m, 1H, CH₂), 4.31-4.40 (m, 3H, CH+CH₂), 4.95 (t, 1H, J=5.6 Hz, CH), 5.11 (br, 1H, CH), 6.63 (d, 1H, J=5.6 Hz, CH), 8.14-8.18 (m, 1H, ArH), 8.92 (d, 1H, J=7.6 Hz, ArH), 9.02 (d, J=6.0 Hz, ArH), 9.27 (s, 1H, ArH).

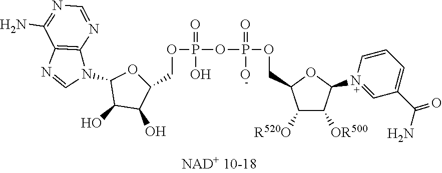

General procedure for the synthesis of NAD⁺10-18: To a stirred solution of Adenosine 5′-monophosphate (5′-AMP) (52 mg, 0.15 mmol, 1.5 eq) in dried DMF (2 mL) were added 1,1-carbonyldiimidazole (CDI) (63 mg, 0.50 mmol, 5 eq) and triethylamine (23 μL, 0.16 mmol. 1.6 eq). The reaction mixture was stirred at room temperature for 14 hours, and then quenched with 0.100 ml dried methanol. The solvent was removed under vacuum and the residue was coevaporated 3 times each with 1.00 ml of dried DMF. The activated 5′-AMP was dissolved in dried DMF (1 mL) and compound 9cc (0.10 mmol, 1.0 eq) was added. After stirring at room temperature for 4 days, H₂O was added to quench the reaction at 0° C. The resulting mixture was continued stirring at room temperature for 24 hours. The reaction was then concentrated in vacuo and the crude product was purified via preparative HPLC. Fractions containing the desired product were concentrated and lyophilized to yield NAD⁺10-18.

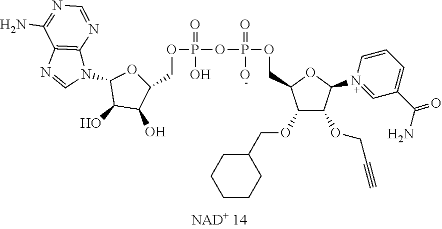

NAD⁺14. A colorless solid; ¹H NMR (400 MHz, D₂O): δ 0.58-0.69 (m, 2H, CH₂), 0.95-1.09 (m, 3H, CH₂), 1.17-1.35 (m, 3H, CH₂), 1.47-1.60 (m, 3H, CH₂), 2.92 (t, 1H, J=2.4 Hz, CH), 3.12 (dd, 1H, J=8.8, 5.6 Hz, CH₂), 3.21-3.25 (m, 1H, CH₂), 4.05-4.13 (m, 2H, CH₂), 4.23-4.29 (m, 5H, CH₂+CH₂+CH), 4.37-4.41 (m, 1H, CH₂), 4.49-4.52 (m, 1H, CH), 4.68 (t, 1H, J=5.6 Hz, CH), 4.91 (t, 1H, J=5.6 Hz, CH), 5.01 (br, 1H, CH), 6.00 (d, 1H, J=6.0 Hz, CH), 6.49 (d, 1H, J=6.8 Hz, CH), 8.04 (dd, 1H, J=8.0, 6.0 Hz, ArH), 8.50 (s, 1H, ArH), 8.80 (d, 1H, J=8.0 Hz, ArH), 8.85 (d, J=6.0 Hz, ArH), 9.04 (s, 1H, ArH)

### Example 4. Synthesis of NAD⁺19

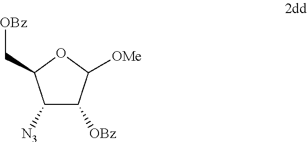

General procedure for the synthesis of compound 2dd: To a solution of ((3 aR,5 S,6R,6aR)-6-azido-2,2-dimethyltetrahydrofuro[2,3-d][1,3]dioxol-5-yl)methyl benzoate (prepared according to the reported method by Nucleosides, Nucleotides and Nucleic Acids, 2013, 32, 646-659) (1.7 mmol) in a mixture of anhydrous MeOH was added AcCl (2.0 eq) at r.t. to offer an intermediate compound A. Then the intermediate compound A was dissolved in DCM (10 mL) and anhydrous pyridine (10 mL) followed by the addition of BzCl (5.1 mmol, 3 eq) at 0° C. Then the reaction mixture was allowed to warm to room temperature. After stirring for 24 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the compound 2dd.

((2S,3R,4R)-3-azido-4-(benzoyloxy)-5-methoxytetrahydrofuran-2-yl)methyl benzoate (2dd). A colorless solid, 56% yield for two steps; ¹H NMR (400 MHz, CDCl₃): δ 3.36 (s, 3H, OCH₃), 4.34 (dd, 1H, J=7.2, 4.4 Hz, CH), 4.46-4.50 (m, 2H, CH₂+CH), 4.66 (dd, 1H, J=13.2, 5.2 Hz, CH₂), 5.05 (s, 1H, CH), 5.51 (d, 1H, J=4.4 Hz, CH), 7.45-7.50 (m, 4H, ArH), 7.57-7.64 (m, 2H, ArH), 8.08-8.13 (m, 4H, ArH); ¹³C NMR (100 MHz, CDCl₃): δ 55.3, 61.2, 64.2, 76.7, 78.9, 106.1, 128.5, 128.6, 128.9, 129.6, 129.8, 130.0, 133.3, 133.7, 165.4, 166.2.

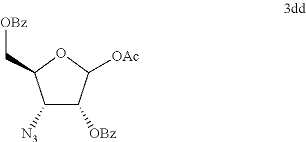

General procedure for the synthesis of compound 3dd: Compound 2dd (1.3 mmol) was dissolved in a mixture of TFA/H₂O (9/1, 15 mL) and the resulting mixture was stirred at room temperature until the reaction complete (monitoring by TLC). Then the reaction was diluted with DCM (60 mL) and the solution was added dropwise to a stirred mixture of ice and saturated aqueous NaHCO₃. Solid NaHCO₃ was added during the addition to maintain a PH of 7. The mixture was extracted with DCM (3×50 mL), and the combined organic extracts was washed with H₂O (50 mL), brine (50 mL), dried over anhydrous Na₂SO₄, filtered, concentrated to give a residue. The residue was dissolved in pyridine (15 mL) and cooled to 0° C. Ac₂O (0.5 mL) was added dropwise and then the resulting mixture was allowed to warm to room temperature. After stirring for 6 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the compound 3dd.

((2S,3R,4R)-5-acetoxy-3-azido-4-(benzoyloxy)tetrahydrofuran-2-yl)methyl benzoate (3dd). A corless oil, 88% yield. ¹H NMR (400 MHz, CDCl₃): δ 1.95 (s, 3H, CH₃), 4.37 (dd, 1H, J=8.4, 4.4 Hz, CH), 4.47-4.52 (m, 2H, CH+CH₂), 4.73 (dd, 1H, J=13.2, 4.4 Hz, CH₂), 5.62 (d, 1H, J=4.4 Hz, CH), 6.32 (s, 1H, CH), 7.44-7.51 (m, 4H, ArH), 7.58-7.65 (m, 2H, ArH), 8.07-8.12 (m, 4H, ArH); ¹³C NMR (100 MHz, CDCl₃): δ 20.8, 60.5, 63.2, 76.2, 80.0, 98.2, 128.5, 128.6, 129.5, 129.7, 129.8, 130.0, 133.5, 133.9, 165.2, 166.0, 168.8.

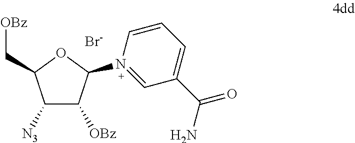

General procedure for the synthesis of compound 4dd: Compound 3dd (0.70 mmol) was dissolved in toluene (10 mL) and cooled to 0° C. HBr (33 wt % in acetic acid) (257 mg, 1.05 mmol, 1.5 eq) was added dropwise and the reaction was stirred at 0° C. for 5 hours. After the starting material was consumed, the reaction was concentrated under reduced pressure to give a residue. The residue was azeotroped with toluene (3×20 mL) to remove remaining acetic acid and dried in vacuo. The crude product and nicotinamide (103 mg, 0.84 mmol, 1.2 eq) was dissolved in CH₃CN (20 mL). The reaction was stirred under Ar gas at room temperature for 24 hours. The reaction was concentrated in vacuo (the temperature was kept below 35° C.) and purified by a flash column chromatography on silica gel to afford the compound 4dd (279 mg, 70%) as a colorless solid.

1-((2R,3R,4R,5 S)-4-azido-3-(benzoyloxy)-5-((benzoyloxy)methyl)tetrahydrofuran-2-yl)-3-carbamoylpyridin-1-ium bromide (4dd). ¹H NMR (400 MHz, CD₃OD): δ 4.77 (m, 1H, CH), 4.89-4.95 (m, 3H, CH+CH₂), 6.03-6.04 (m, 1H, CH), 6.69 (d, 1H, J=1.6 Hz, CH), 7.49-7.53 (m, 2H, ArH), 7.56-7.60 (m, 2H, ArH), 7.63-7.68 (m, 1H, ArH), 7.70-7.75 (m, 1H, ArH), 8.06-8.09 (m, 2H, ArH), 8.19-8.21 (m, 2H, ArH), 8.24 (dd, 1H, J=8.0, 6.4 Hz, ArH), 9.03-9.06 (m, 1H, ArH), 9.41 (d, 1H, J=6.4 Hz, ArH), 9.66 (s, 1H, ArH); ¹³C NMR (100 MHz, CD₃OD): δ 60.3, 63.8, 79.9, 84.3, 99.3, 129.56, 129.60, 129.9, 130.0, 130.5, 130.8, 131.2, 134.9, 135.5, 136.0, 142.3, 143.9, 147.0, 164.8, 167.3, 167.5.

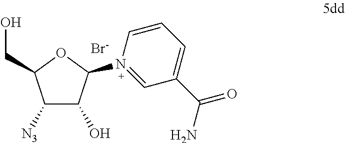

General procedure for the synthesis of compound 5dd: Compound 4dd (0.45 mmol) was dissolved in ammonia (18 mL, 7 N in MeOH) and the reaction was stirred at −10° C. for 48 hours. The reaction was concentrated under reduced pressure and the crude product was dissolved in MeOH (0.5 mL). Addition of ethyl ether (10 mL) resulted in ppt of the desired product. The procedure was repeated three times to yield the desired product 5dd (91 mg, 63%) as a colorless solid.

1-((2R,3R,4S,5S)-4-azido-3-hydroxy-5-(hydroxymethyl)tetrahydrofuran-2-yl)-3-carbamoyl-pyridin-1-ium bromide (5dd). A colorless solid, 63% yield; ¹H NMR (400 MHz, D₂O): δ 3.91 (dd, 1H, J=13.2, 2.8 Hz, CH₂), 4.08 (dd, 1H, J=13.2, 2.8 Hz, CH₂), 4.35 (t, 1H, J=5.2 Hz, CH), 4.50-4.52 (m, 1H, CH), 4.79 (m, 1H, CH, overlap with solvent residue peak), 6.27 (d, 1H, J=4.0 Hz, CH), 8.28 (dd, 1H, J=8.0, 6.8 Hz, ArH), 8.98 (d, 1H, J=8.0 Hz, ArH), 9.27 (d, 1H, J=6.8 Hz, ArH), 9.61 (s, 1H, ArH); ¹³C NMR (100 MHz, D₂O): δ 60.0, 60.7, 77.6, 85.5, 99.5, 128.3, 133.9, 140.3, 142.6, 145.7, 165.7.

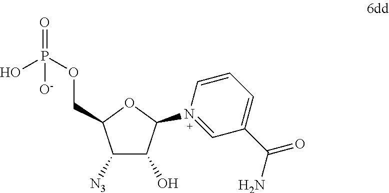

General procedure for the synthesis of compound 6dd: To a stirred solution of compound 5dd (0.27 mmol) in trimethylphosphate (2 mL) was added P(O)Cl₃ (175 μL, 1.89 mmol, 7 eq) at 0° C. and the resulting mixture was stirred at 0° C. for 6 hours. A few drops H₂O was then added to quench the reaction. Trimethylphosphate was removed by extraction with ethyl ether (3×20 ml). The remaining trimethylphosphate was removed by a second extraction with THF (5 ml). The aqueous layer was concentrated in vacuo and the crude product was dissolved in MeOH (0.5 mL). Addition of ethyl ether (10 mL) resulted in ppt of the desired product. The procedure was repeated three times to yield the desired product 6dd (52 mg, 72%) as a colorless solid.

((2S,3S,4R,5R)-3-azido-5-(3-carbamoylpyridin-1-ium-1-yl)-4-hydroxytetrahydrofuran-2-yl)methyl hydrogen phosphate (6dd). A colorless solid, 72% yield; ¹H NMR (400 MHz, D₂O): δ 4.15 (ddd, 1H, J=12.0, 4.8, 2.4 Hz, CH₂), 4.32 (ddd, 1H, J=12.0, 4.4, 2.4 Hz, CH₂), 4.49 (dd, 1H, J=5.6, 2.8 Hz, CH), 4.64 (t, 1H, J=2.4 Hz, CH), 4.83 (t, 1H, J=5.6 Hz, CH, overlap with solvent residue peak), 6.22 (d, 1H, J=5.6 Hz, CH), 8.29 (dd, 1H, J=8.0, 6.0 Hz, ArH), 8.98 (d, 1H, J=8.0 Hz, ArH), 9.26 (d, 1H, J=6.0 Hz, ArH), 9.44 (s, 1H, ArH); ¹³C NMR (100 MHz, D₂O): δ 62.3, 64.3 (d, J=4.7 Hz), 77.8, 85.1 (d, J=8.7 Hz), 99.3, 128.5, 133.9, 139.8, 142.4, 146.0, 165.7.

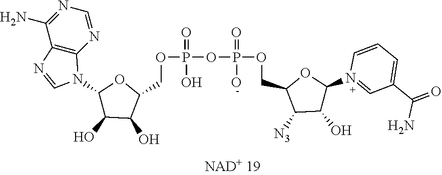

General procedure for the synthesis of NAD⁺19: To a stirred solution of Adenosine 5′-monophosphate (5′-AMP) (52 mg, 0.15 mmol, 1.5 eq) in dried DMF (2 mL) were added 1,1-carbonyldiimidazole (CDI) (63 mg, 0.50 mmol, 5 eq) and triethylamine (23 μL, 0.16 mmol. 1.6 eq). The reaction mixture was stirred at room temperature for 14 hours, and then quenched with 0.100 ml dried methanol. The solvent was removed under vacuum and the residue was coevaporated 3 times each with 1.00 ml of dried DMF. The activated 5′-AMP was dissolved in dried DMF (1 mL) and compound 6dd (0.10 mmol, 1.0 eq) was added. After stirring at room temperature for 4 days, H₂O was added to quench the reaction at 0° C. The resulting mixture was continued stirring at room temperature for 24 hours. The reaction was then concentrated in vacuo and the crude product was purified via preparative HPLC. Fractions containing the desired product were concentrated and lyophilized to yield NAD⁺19.

NAD⁺19. A colorless solid, 45% yield; ¹H NMR (400 MHz, D₂O): δ 4.18-4.25 (m, 2H, CH₂), 4.38-4.41 (m, 2H, CH₂), 4.49-4.53 (m, 2H, 2CH), 4.59 (br, 1H, CH), 4.71 (t, 1H, J=5.6 Hz, CH), 4.68-4.80 (1H, CH, overlapped with solvent residue peak), 4.83 (t, 1H, J=5.6 Hz, CH), 6.12 (d, 1H, J=5.2 Hz, CH), 6.16 (d, 1H, J=5.2 Hz, CH), 8.27-8.30 (m, 1H, ArH), 8.39 (s, 1H, ArH), 8.58 (s, 1H, ArH), 8.94 (d, 1H, J=8.0 Hz, ArH), 9.25 (d, 1H, J=6.4 Hz, ArH), 9.40 (s, 1H, ArH); ¹³C NMR (100 MHz, D₂O): δ 62.3, 65.02 (d, J=5.3 Hz), 65.06 (d, J=3.6 Hz), 70.1, 74.5, 77.7, 84.0 (d, J=8.3 Hz), 84.9 (d, J=8.9 Hz), 87.7, 99.3, 118.3, 128.6, 133.8, 139.8, 142.2, 142.5, 145.0, 146.1, 148.2, 149.9, 165.4, 165.8; HRMS (ESI) Calcd. For C₂₁H₂₇N₁₀O₃P₂⁺ (M+H)⁺ requires 689.1234, Found: 689.1226.

### Example 5. Synthesis of NAD⁺20 and NAD⁺26

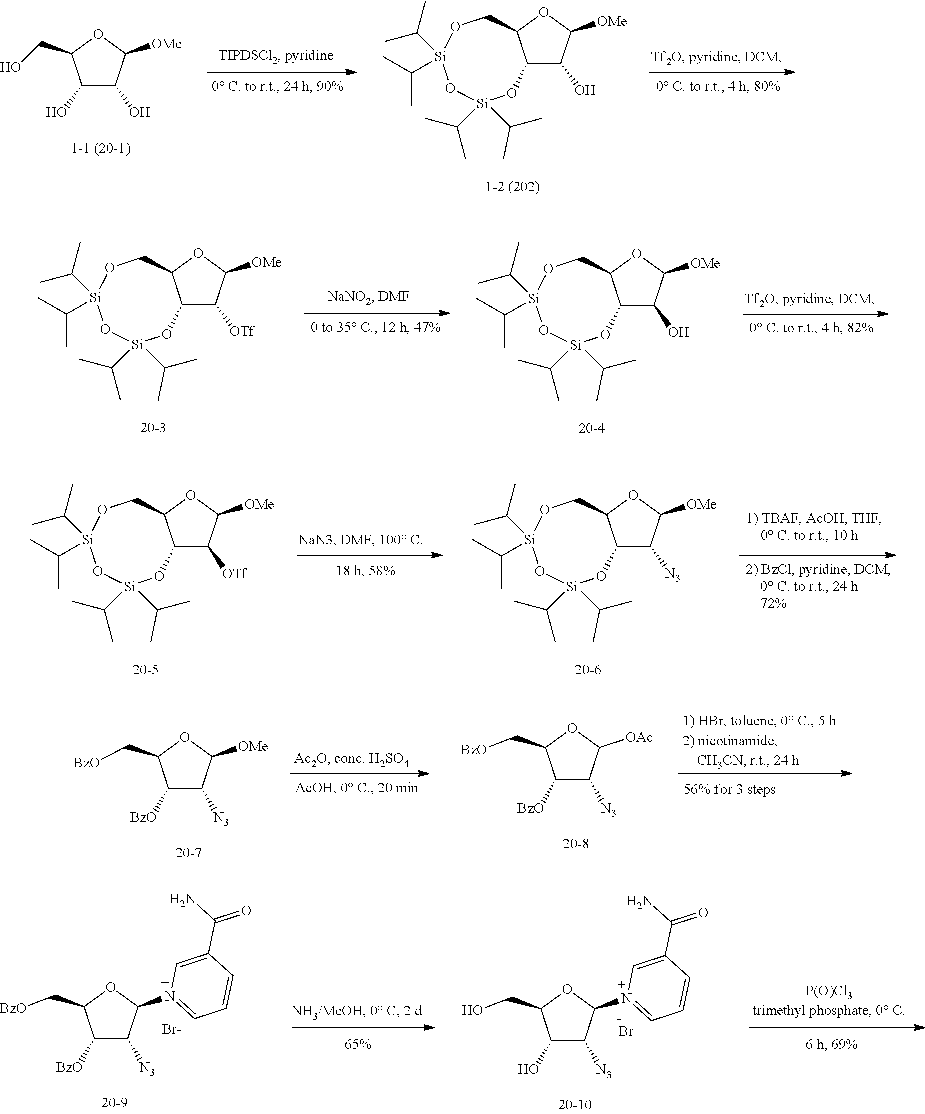

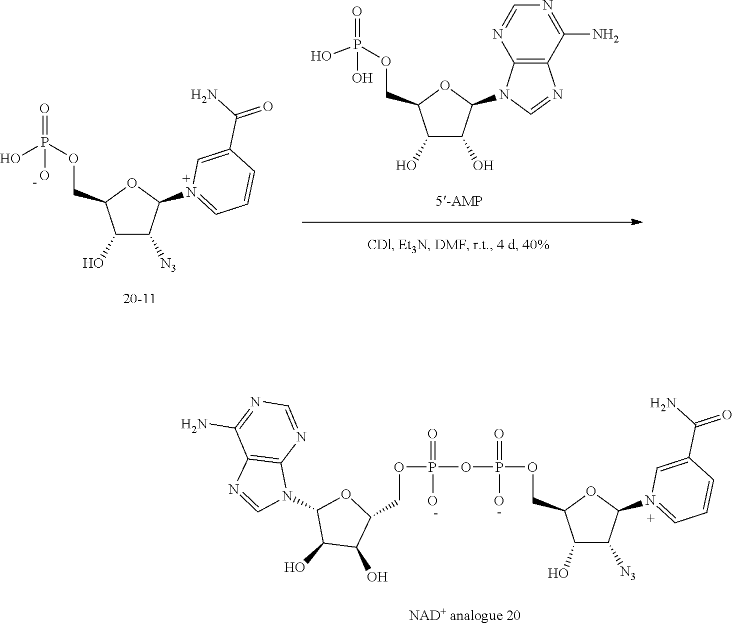

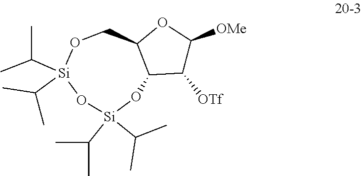 [SMILES: CO[C@@H]1O[C@@H]2CO[Si](C(C)C)(C(C)C)O[Si](C(C)C)(C(C)C)O[C@H]2[C@H]1OS(=O)(=O)C(F)(F)F]

General procedure for the synthesis of (6aR,8R,9R,9aR)-2,2,4,4-tetraisopropyl-8-methoxytetrahydro-6H-furo[3,2-f][1,3,5,2,4]triox-adisilocin-9-yl trifluoromethanesulfonate (20-3): The compound 20-2 (1.21 g, 3.0 mmol) was dissolved in DCM followed by the addition of pyridine (711 mg, 9.0 mmol, 3 eq) and Tf₂O (1.3 g, 4.5 mmol, 1.5 eq) at 0° C. The reaction mixture was allowed to warm to room temperature. After stirring at this temperature for 4 hours, the reaction mixture was diluted with EtOAc (100 mL) and quenched with water (2 mL), and the organic phase was washed with water (5×50 mL), dried over anhydrous Na₂SO₄, filtered and concentrated to give a residue. The residue was purified by a flash column chromatography on silica gel to afford the corresponding (6aR,8R,9R,9aR)-2,2,4,4-tetraisopropyl-8-methoxytetrahydro-6H-furo[3,2-f][1,3,5,2,4]triox-adisilocin-9-Y¹ trifluoromethanesulfonate (20-3) (1.29 g, 80%) as a colorless oil.

(6aR,8R,9R,9aR)-2,2,4,4-tetraisopropyl-8-methoxytetrahydro-6H-furo[3,2-f][1,3,5,2,4]triox-adisilocin-9-yl trifluoromethanesulfonate (20-3). A colorless oil, 80% yield; ¹H NMR (400 MHz, CDCl₃): δ 0.98-1.10 (m, 28H, 8CH₃+4CH), 3.36 (s, 3H, OCH₃), 3.87 (dd, 1H, J=12.8, 7.2 Hz, CH₂), 3.99-4.05 (m, 2H, CH₂+CH), 4.63 (dd, 1H, J=7.2, 4.4 Hz, CH), 4.93 (s, 1H, CH), 4.98 (d, 1H, J=4.4 Hz, CH); ¹³C NMR (100 MHz, CDCl₃): δ 12.68, 12.72, 13.1, 13.2, 16.68, 16.71, 16.87, 16.90, 17.27, 17.32, 17.4, 55.2, 63.8, 71.7, 81.3, 88.9, 103.8, 118.6 (q, J=317.0 Hz).

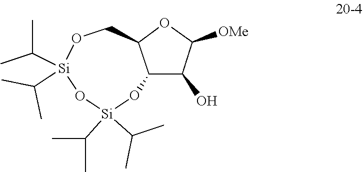 [SMILES: CO[C@@H]1O[C@@H]2CO[Si](C(C)C)(C(C)C)O[Si](C(C)C)(C(C)C)O[C@H]2[C@@H]1O]

General procedure for the synthesis of (6aR,8R,9S,9aS)-2,2,4,4-tetraisopropyl-8-methoxytetrahydro-6H-furo[3,2-f][1,3,5,2,4]trioxadisilocin-9-ol (20-4): The compound 20-3 (1.24 g, 2.3 mmol) was dissolved in DMF (20 mL) and NaNO₂ (793 mg, 11.5 mmol, 5 eq) was added to the mixture at r.t. Then the resulting mixture was heated to 35° C. After stirring at this temperature for 12 hours, the reaction mixture was diluted with EtOAc (100 mL), and the organic phase was washed with water (5×50 mL), dried over anhydrous Na₂SO₄, filtered and concentrated to give a residue. The residue was purified by a flash column chromatography on silica gel to afford the corresponding compound (6aR,8R,9S,9aS)-2,2,4,4-tetraisopropyl-8-methoxytetrahydro-6H-furo[3,2-f][1,3,5,2,4]trioxadisilocin-9-ol. (20-4) (440 mg, 47%) as a colorless oil.

(6aR,8R,9S,9aS)-2,2,4,4-tetraisopropyl-8-methoxytetrahydro-6H-furo[3,2-f][1,3,5,2,4]triox-adisilocin-9-ol (20-4). A colorless oil, 47% yield; ¹H NMR (400 MHz, CDCl₃): δ 0.99-1.11 (m, 28H, 8CH₃+4CH), 2.25 (d, 1H, J=9.6 Hz, OH), 3.40 (s, 3H, OCH₃), 3.76 (dd, 1H, J=10.8, 8.8 Hz, CH₂), 3.83-3.87 (m, 1H, CH), 3.96 (dd, 1H, J=10.8, 3.2 Hz, CH₂), 4.10-4.15 (m, 1H, CH), 4.21 (dd, 1H, J=7.6, 6.0 Hz, CH), 4.74 (d, 1H, J=4.0 Hz, CH); ¹³C NMR (100 MHz, CDCl₃): δ 12.5, 12.8, 13.3, 13.4, 16.97, 16.99, 17.05, 17.38, 17.40, 17.44, 17.53, 55.2, 66.0, 78.7, 79.6, 82.1, 101.3.

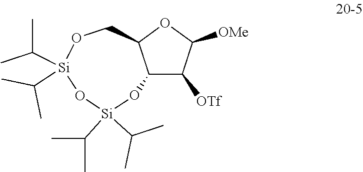 [SMILES: CO[C@@H]1O[C@@H]2CO[Si](C(C)C)(C(C)C)O[Si](C(C)C)(C(C)C)O[C@H]2[C@@H]1OS(=O)(=O)C(F)(F)F]

General procedure for the synthesis of (6aR,8R,9S,9aR)-2,2,4,4-tetraisopropyl-8-methoxytetrahydro-6H-furo[3,2-f][1,3,5,2,4]trioxadisilocin-9-yl trifluoromethanesulfonate (20-5): To a stirred solution of compound 20-4 (407 mg, 1.0 mmol) in DCM (15 mL) were added pyridine (237 mg, 3.0 mmol, 3 eq) and Tf₂O (433 mg, 1.5 mmol, 1.5 eq) at 0° C. The reaction mixture was allowed to warm to room temperature. After stirring at this temperature for 4 hours, the reaction mixture was diluted with EtOAc (100 mL), and the organic phase was washed with water (5×50 mL), dried over anhydrous Na₂SO₄, filtered and concentrated to give a residue. The residue was purified by a flash column chromatography on silica gel to afford the corresponding compound (6aR,8R,9S,9aR)-2,2,4,4-tetraisopropyl-8-methoxytetrahydro-6H-furo[3,2-f][1,3,5,2,4]trioxadisilocin-9-yl trifluoromethanesulfonate (20-5) (442 mg, 82%) as a colorless oil.

(6aR,8R,9S,9aR)-2,2,4,4-tetraisopropyl-8-methoxytetrahydro-6H-furo[3,2-f][1,3,5,2,4]trio-xadisilocin-9-yl trifluoromethanesulfonate (20-5). A colorless oil, 82% yield; ¹H NMR (400 MHz, CDCl₃): δ 0.99-1.11 (m, 28H, 8CH₃+4CH), 3.40 (s, 3H, OCH₃), 3.81 (dd, 1H, J=10.8, 9.2 Hz, CH₂), 3.90-3.95 (m, 1H, CH), 3.98 (dd, 1H, J=10.8, 3.2 Hz, CH₂), 4.70 (dd, 1H, J=7.6, 5.6 Hz, CH), 4.92 (d, 1H, J=4.4 Hz, CH), 4.97 (dd, 1H, J=7.6, 4.4 Hz, CH); ¹³C NMR (100 MHz, CDCl₃): δ 12.4, 12.8, 13.18, 13.24, 16.69, 16.72, 16.76, 16.77, 17.31, 17.33, 17.37, 17.48, 55.4, 66.0, 76.0, 81.3, 88.9, 99.3, 118.5 (q, J=317.9);

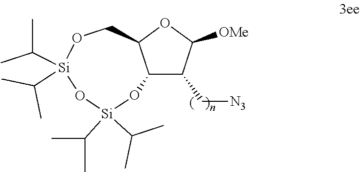

General procedure for the synthesis of 3ee: If n is, for example, 0, the compound 20-5 or ((6aR,8R,9R,9aS)-2,2,4,4-tetraisopropyl-8-methoxytetrahydro-6H-furo[3,2-f][1,3,5,2,4]trioxadisilocin-9-yl)methyl trifluoromethanesulfonate (808 mg, 1.5 mmol) was dissolved in DMF (20 mL) and NaN₃ (488 mg, 7.5 mmol, 5 eq) was added to the mixture at room temperature. After stirring at 100° C. for 18 hours, the reaction mixture was diluted with EtOAc (100 mL), and the organic phase was washed with water (5×50 mL), dried over anhydrous Na₂SO₄, filtered and concentrated to give a residue. The residue was purified by a flash column chromatography on silica gel to afford the corresponding compound 3ee.

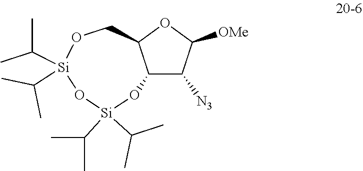 [SMILES: CO[C@@H]1O[C@@H]2CO[Si](C(C)C)(C(C)C)O[Si](C(C)C)(C(C)C)O[C@H]2[C@H]1N=[N+]=[N-]]

(6aR,8R,9R,9aS)-9-azido-2,2,4,4-tetraisopropyl-8-methoxytetrahydro-6H-furo[3,2-f][1,3,5,2,4]trioxadisilocine (20-6). A colorless oil, 376 mg, 58% yield; ¹H NMR (400 MHz, CDCl₃): δ 1.01-1.11 (m, 28H, 8CH₃+4CH), 3.29 (s, 3H, OCH₃), 3.79 (dd, 1H, J=12.0, 8.4 Hz, CH₂), 3.88 (d, 1H, J=5.2 Hz, CH), 3.99-4.04 (m, 2H, CH+CH₂), 4.61 (s, 1H, CH), 4.74 (dd, 1H, J=7.2, 5.2 Hz, CH); ¹³C NMR (100 MHz, CDCl₃): δ 12.7, 13.2, 13.3, 16.84, 16.85, 17.1, 17.27, 17.30, 17.34, 17.36, 17.45, 54.8, 65.0, 67.0, 75.8, 81.7, 105.2.

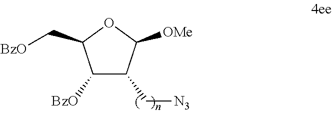

General procedure for the synthesis of 4ee: To a 0° C. solution of compound 3ee (0.8 mmol) in anhydrous THF (1 mL) was added AcOH (72 mg, 1.2 mmol, 1.5 eq) followed by the addition of TBAF (1.2 mL, 1.2 mmol, 1.0 M in THF, 1.5 eq). Then reaction mixture was allowed to warm to room temperature. After stirring at this temperature for 10 hours, the reaction mixture was concentrated under reduced pressure to give a residue. The residue was dissolved in a mixture of anhydrous DCM (10 mL) and anhydrous pyridine (10 mL) was added BzCl (277 μL, 2.4 mmol, 3 eq) at 0° C. Then the reaction mixture was allowed to warm to room temperature. After stirring for 24 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the corresponding compound 4ee.

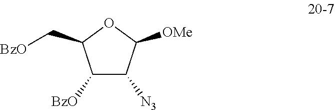

(2R,3S,4R,5R)-4-azido-2-((benzoyloxy)methyl)-5-methoxytetrahydrofuran-3-Y¹ benzoate. A colorless oil, 229 mg, 72% yield; ¹H NMR (400 MHz, CDCl₃): δ 3.36 (s, 3H, OCH₃), 4.32 (d, 1H, J=5.2 Hz, CH), 4.48 (dd, 1H, J=13.2, 6.4 Hz, CH₂), 4.60-4.65 (m, 2H, CH+CH₂), 4.95 (s, 1H, CH), 5.72 (t, 1H, J=7.2, 5.2 Hz, CH), 7.38 (t, 2H, J=8.0 Hz, ArH), 7.46 (t, 2H, J=7.6 Hz, ArH), 7.51-7.55 (m, 1H, ArH), 7.58-7.62 (m, 1H, ArH), 8.05 (d, 2H, J=8.0 Hz, ArH), 8.08 (d, 2H, J=7.6 Hz, ArH); ¹³C NMR (100 MHz, CDCl₃): δ 55.3, 64.6, 65.3, 74.2, 78.6, 106.7, 128.3, 128.51, 128.54, 129.58, 129.63, 129.9, 133.1, 133.7.

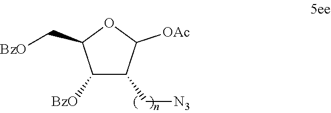

General procedure for the synthesis of 5ee: To a stirred solution of compound 4ee (0.5 mmol) in a mixture of AcOH (4 mL) and Ac₂O (1.0 mL) was add conc. H₂SO₄ (40 μL) at 0° C. The resulting mixture was stirred at the same temperature until the reaction complete (monitoring by TLC, about 20 min). Then the reaction was diluted with DCM (60 mL) and the solution was added dropwise to a stirred mixture of ice and saturated aqueous NaHCO₃. Solid NaHCO₃ was added during the addition to maintain a PH of 7. The mixture was extracted with DCM (3×50 mL), and the combined organic extracts was washed with H₂O (50 mL), brine (50 mL), dried over anhydrous Na₂SO₄, filtered, concentrated to give a residue. The residue was used directly for next step without further purification.

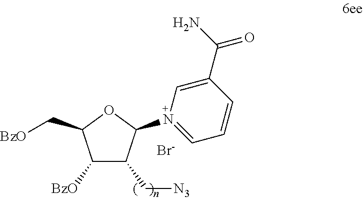

General procedure for the synthesis of 6ee: The residue 5ee from last step was dissolved in toluene (10 mL) and cooled to 0° C. HBr (33 wt % in acetic acid) (183 mg, 0.75 mmol, 1.5 eq) was added dropwise and the reaction was stirred at 0° C. for 5 hours. After the starting material was consumed, the reaction was concentrated under reduced pressure to give a residue. The residue was azeotroped with toluene (3×20 mL) to remove remaining acetic acid and dried in vacuo. The crude product and nicotinamide (73 mg, 0.6 mmol, 1.2 eq) was dissolved in CH₃CN (10 mL). The reaction was stirred under Ar gas at room temperature for 24 hours. The reaction was concentrated in vacuo (the temperature was kept below 35° C.) and purified by a flash column chromatography on silica gel to afford the compound 6ee.

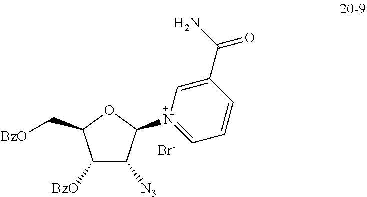

1-((2R,3R,4S,5R)-3-azido-4-(benzoyloxy)-5-((benzoyloxy)methyl)tetrahydrofuran-2-yl)-3-carbamoylpyridin-1-ium bromide. A colorless solid, 158 mg, 56% yield for 3 steps; ¹H NMR (400 MHz, CD₃OD): δ 4.72 (dd, 1H, J=12.4, 4.8 Hz, CH₂), 4.78 (dd, 1H, J=12.4, 4.8 Hz, CH₂), 5.57-5.62 (m, 2H, 2CH), 6.09 (dd, 1H, J=5.2, 2.0 Hz, CH), 7.09 (d, 1H, J=6.4 Hz, CH), 7.42 (t, 2H, J=8.0 Hz, ArH), 7.48 (t, 2H, J=8.0 Hz, ArH), 7.57-7.62 (m, 2H, ArH), 7.76-7.78 (m, 2H, ArH), 8.08-8.10 (m, 2H, ArH), 8.31 (dd, 1H, J=8.0, 6.4 Hz, ArH), 9.09 (d, 1H, J=8.0 Hz, ArH), 9.40 (d, 1H, J=6.4 Hz, ArH), 9.59 (s, 1H, ArH); ¹³C NMR (100 MHz, CD₃OD): δ 64.7, 65.2, 74.4, 86.3, 97.0, 128.4, 129.6, 129.81, 129.85, 130.7, 130.8, 134.70, 134.78, 135.1, 142.2, 145.2, 146.9, 164.8, 166.3, 167.4.

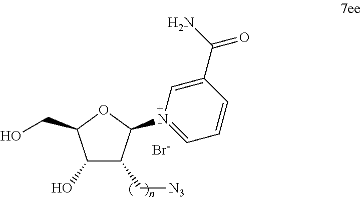

General procedure for the synthesis of 7ee: Compound 6ee (0.22 mmol) was dissolved in ammonia (15 mL, 7 N in MeOH) and the reaction was stirred at 0° C. for 48 hours. The reaction was concentrated under reduced pressure and the crude product was dissolved in MeOH (0.5 mL). Addition of ethyl ether (10 mL) resulted in ppt of the desired product. The procedure was repeated three times to yield the desired product 7ee.

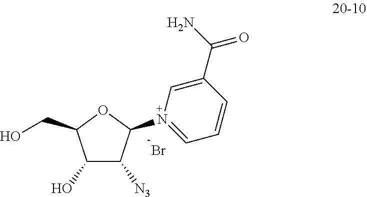

1-((2R,3R,4 S,5R)-3-azido-4-hydroxy-5-(hydroxymethyl)tetrahydrofuran-2-yl)-3-carbamo-ylpyridin-1-ium bromide. A colorless solid, 52 mg, 65% yield; ¹H NMR (400 MHz, D₂O): δ 3.78 (dd, 1H, J=12.8, 4.4 Hz, CH₂), 3.91 (dd, 1H, J=12.8, 3.2 Hz, CH₂), 4.66 (t, 1H, J=4.0 Hz, CH), 4.79-4.82 (m, 1H, CH, overlap with water), 5.03 (m, 1H, CH), 6.68 (d, 1H, J=6.4 Hz, CH), 8.21-8.24 (m, 1H, ArH), 8.97 (d, 1H, J=8.4 Hz, ArH), 9.10 (d, 1H, J=6.0 Hz, ArH), 9.31 (s, 1H, ArH); ¹³C NMR (100 MHz, D₂O): δ 60.6, 64.4, 70.8, 88.8, 95.5, 127.2, 132.7, 141.0, 143.5, 145.5, 165.7.

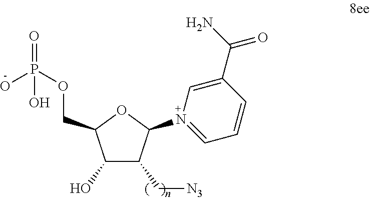

General procedure for the synthesis of 8ee: To a stirred solution of compound 7ee (0.12 mmol) in trimethylphosphate (1.5 mL) was added P(O)Cl₃ (78 μL, 0.84 mmol, 7 eq) at 0° C. and the resulting mixture was stirred at 0° C. for 6 hours. A few drops H₂O was then added to quench the reaction. Trimethylphosphate was removed by extraction with ethyl ether (3×20 ml). The remaining trimethylphosphate was removed by a second extraction with THF (5 ml). The aqueous layer was concentrated in vacuo and the crude product was dissolved in MeOH (0.5 mL). Addition of ethyl ether (10 mL) resulted in ppt of the desired product. The procedure was repeated three times to yield the desired product 8ee.

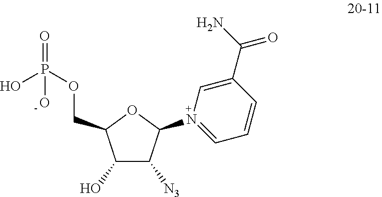

1-((2R,3R,4S,5R)-3-azido-4-hydroxy-5-(hydroxymethyl)tetrahydrofuran-2-yl)-3-carbamoy-lpyridin-1-ium bromide (20-11). A colorless oil, 30 mg, 69% yield; ¹H NMR (400 MHz, D₂O): δ 4.08 (ddd, 1H, J=12.0, 5.6, 2.8 Hz, CH₂), 4.17 (ddd, 1H, J=12.0, 5.6, 2.8 Hz, CH₂), 4.72 (dd, 1H, J=4.8, 2.4 Hz, CH), 4.90-4.95 (m, 1H, CH), 5.07 (t, 1H, J=5.6 Hz, CH), 6.67 (d, 1H, J=5.6 Hz, CH), 8.17-8.21 (m, 1H, ArH), 8.94 (d, 1H, J=8.4 Hz, ArH), 9.08 (d, 1H, J=6.0 Hz, ArH), 9.31 (s, 1H, ArH); ¹³C NMR (100 MHz, D₂O): δ 64.3, 64.5 (d, J=5.0 Hz), 70.9, 87.8 (d, J=7.9 Hz), 95.8, 127.1, 132.7, 141.1, 143.5, 145.5, 165.7.

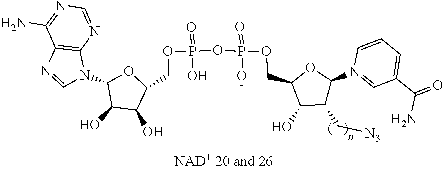

General procedure for the synthesis of (NAD⁺ analogue 20 and 26): To a stirred solution of Adenosine 5′-monophosphate (5′-AMP) (52 mg, 0.15 mmol, 1.5 eq) in dried DMF (2 mL) were added 1,1-carbonyldiimidazole (CDI) (63 mg, 0.50 mmol, 5 eq) and triethylamine (23 μL, 0.16 mmol. 1.6 eq). The reaction mixture was stirred at room temperature for 14 hours, and then quenched with 0.100 ml dried methanol. The solvent was removed under vacuum and the residue was coevaporated 3 times each with 1.00 ml of dried DMF. The activated 5′-AMP was dissolved in dried DMF (1 mL) and compound 8ee (0.10 mmol, 1.0 eq) was added. After stirring at room temperature for 4 days, H₂O was added to quench the reaction at 0° C. The resulting mixture was continued stirring at room temperature for 24 hours. The reaction was then concentrated in vacuo and the crude product was purified via preparative HPLC. Fractions containing the desired product were concentrated and lyophilized to yield the NAD⁺20 and 26.

NAD⁺20. A colorless oil, 69% yield; ¹H NMR (400 MHz, D₂O): δ 4.15-4.19 (m, 1H, CH), 4.24-4.30 (m, 3H, CH₂+CH₂), 4.40-4.41 (m, 1H, CH), 4.53 (dd, 1H, J=5.2, 4.0 Hz, CH), 4.73-4.77 (m, 2H, 2CH), 4.92 (br, 1H, CH), 5.14 (dd, 1H, J=6.4, 5.2 Hz, CH), 6.11 (d, 1H, J=5.6 Hz, CH), 6.68 (d, 1H, J=6.0 Hz, CH), 8.18 (dd, 1H, J=8.0, 6.4 Hz, ArH), 8.40 (s, 1H, ArH), 8.62 (s, 1H, ArH), 8.92-8.94 (m, 1H, ArH), 9.08 (d, 1H, J=6.4 Hz, ArH), 9.27 (s, 1H, ArH); HRMS (ESI) Calcd. For C₂₁H₂₇N₁₀O₃P₂⁺ (M+H)⁺ requires 689.1234, Found: 689.1250.

### Example 6. Synthesis of NAD⁺22-23

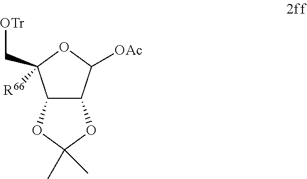

Compound 2ff was prepared according to the reported method (Synlett 2007, No. 20, 3149-3154). If the target compound is, for example NAD⁺22, then R⁶⁶ is —C≡CH. If the target compound is, for example NAD⁺23, then R⁶⁶ is —CH₂CH₂C≡C.

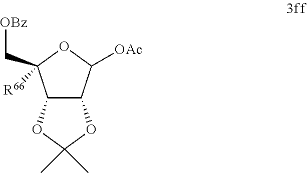

General procedure for the synthesis of compound 3ff: To a solution of compound 2ff (2.0 mmol) in a mixture of anhydrous Et₂O (15 ml) was added HCOOH (15 mL) at 0° C. Then the resulting mixture was allowed to warm to r.t. and stirred overnight. Removing the solvent under reduced pressure to offer an intermediate compound A. Then the compound A was dissolved in DCM (10 mL) and anhydrous pyridine (10 mL) followed by the addition of BzCl (6.0 mmol, 3 eq) at 0° C. Then the reaction mixture was allowed to warm to room temperature. After stirring for 24 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the corresponding compound 3ff.

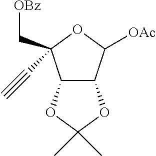

((3 aS,4R,6aR)-6-acetoxy-4-ethynyl-2,2-dimethyltetrahydrofuro[3,4-d][1,3]dioxol-4-yl)methyl benzoate. ¹H NMR (400 MHz, CDCl₃): δ 1.40 (s, 3H, CH₃), 1.61 (s, 3H, CH₃), 1.98 (s, 3H, CH₃), 2.77 (s, 1H, CH), 4.44 (d, 1H, J=11.2 Hz, CH), 4.52 (d, 1H, J=11.2 Hz, CH₂), 4.86 (s, 2H, 2CH), 6.31 (s, 1H, CH), 7.46 (t, 2H, J=7.6 Hz, ArH), 7.58-7.62 (m, 1H, ArH), 8.09 (dd, 2H, J=7.6, 1.2 Hz, ArH).

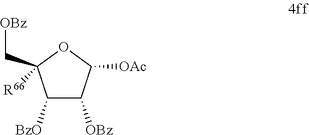

General procedure for the synthesis of compound 4ff: Compound 3ff (1.3 mmol) was dissolved in a mixture of TFA/H₂O (9/1, 15 mL) and the resulting mixture was stirred 0° C. until the reaction complete (monitoring by TLC). Then the reaction was diluted with DCM (60 mL) and the solution was added dropwise to a stirred mixture of ice and saturated aqueous NaHCO₃. Solid NaHCO₃ was added during the addition to maintain a PH of 7. The mixture was extracted with DCM (3×50 mL), and the combined organic extracts was washed with H₂O (50 mL), brine (50 mL), dried over anhydrous Na₂SO₄, filtered, concentrated to give a residue. The residue was dissolved in pyridine (15 mL) and cooled to 0° C. BzCl (3.9 mmol, 3 eq) was added dropwise and then the resulting mixture was allowed to warm to room temperature. After stirring for 6 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the corresponding compound 4ff.

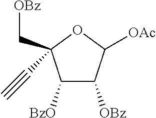

(2R,3S,4R)-5-acetoxy-2-((benzoyloxy)methyl)-2-ethynyltetrahydrofuran-3,4-diyl dibenzoate. ¹H NMR (400 MHz, CDCl₃): δ 1.88 (s, 3H, CH₃), 2.79 (s, 1H, CH), 4.54 (d, 1H, J=12.0 Hz, CH), 4.87 (d, 1H, J=12.0 Hz, CH₂), 5.83 (d, 1H, J=8.8 Hz, CH), 6.04 (d, 1H, J=8.8 Hz, CH) 6.48 (s, 1H, CH), 7.32-7.36 (m, 2H, ArH), 7.39-7.47 (m, 4H, ArH), 7.51-7.63 (m, 3H, ArH), 7.94 (dd, 2H, J=8.0, 1.2 Hz, ArH), 8.09-8.13 (m, 4H, ArH).

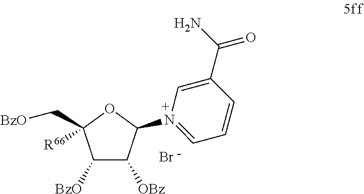

General procedure for the synthesis of compound 5ff: TMSOTf (0.55 mmol, 5.5 eq) was added to a solution of nicotinamide (0.30 mmol, 3 eq) in CH₃CN (3 mL) at 0° C. Then the reaction was allowed to warm to room temperature and stirred until all of the nicotinamide had dissolved. Then a solution of compound 4ff (0.10 mmol) was added to the solution of nicotinamide with TMSOTf at 0° C. Then the reaction was stirred under Ar gas at room temperature for 24 hours. The reaction was concentrated in vacuo (the temperature was kept below 35° C.) and purified by a flash column chromatography on silica gel to afford the compound 5ff.

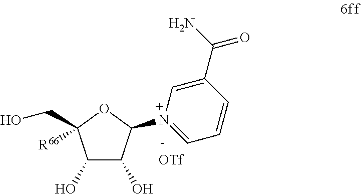

General procedure for the synthesis of compound 6ff: Compound 5ff (0.45 mmol) was dissolved in ammonia (18 mL, 7 N in MeOH) and the reaction was stirred at −10° C. for 48 hours. The reaction was concentrated under reduced pressure and the crude product was dissolved in MeOH (0.5 mL). Addition of ethyl ether (10 mL) resulted in ppt of the desired product. The procedure was repeated three times to yield the desired product 6ff.

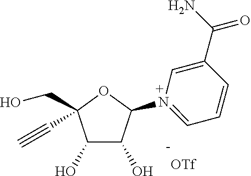

3-carbamoyl-1-((2R,3R,4S,5R)-5-ethynyl-3,4-dihydroxy-5-(hydroxymethyl)tetrahydrofuran-2-yl)pyridin-1-ium trifluoromethanesulfonate. ¹H NMR (400 MHz, D₂O): δ 3.28 (s, 1H, CH), 3.95 (d, 1H, J=12.8 Hz, CH), 4.03 (d, 1H, J=12.8 Hz, CH₂), 4.43 (d, 1H, J=5.6 Hz, CH), 4.61 (t, 1H, J=5.6 Hz, CH) 6.33 (d, 1H, J=4.4 Hz, CH), 8.28 (dd, 1H, J=8.0, 6.4 Hz, ArH), 8.98-9.01 (m, 1H, ArH), 9.25 (d, 1H, J=6.4 Hz, ArH), 9.56 (s, 1H, ArH).

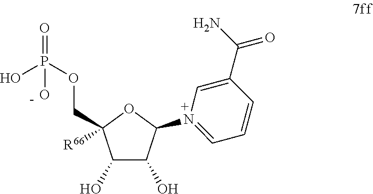

General procedure for the synthesis of compound 7ff: To a stirred solution of compound 6ff (0.27 mmol) in trimethylphosphate (2 mL) was added P(O)Cl₃ (175 μL, 1.89 mmol, 7 eq) at 0° C. and the resulting mixture was stirred at 0° C. for 6 hours. A few drops H₂O was then added to quench the reaction. Trimethylphosphate was removed by extraction with ethyl ether (3×20 ml). The remaining trimethylphosphate was removed by a second extraction with THF (5 ml). The aqueous layer was concentrated in vacuo and the crude product was dissolved in MeOH (0.5 mL). Addition of ethyl ether (10 mL) resulted in ppt of the desired product. The procedure was repeated three times to yield the desired product 7ff.

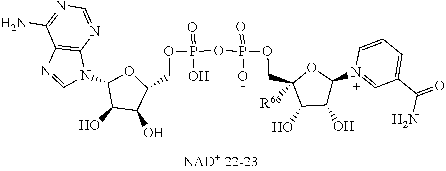

General procedure for the synthesis of NAD⁺23: To a stirred solution of Adenosine 5′-monophosphate (5′-AMP) (52 mg, 0.15 mmol, 1.5 eq) in dried DMF (2 mL) were added 1,1-carbonyldiimidazole (CDI) (63 mg, 0.50 mmol, 5 eq) and triethylamine (23 μL, 0.16 mmol. 1.6 eq). The reaction mixture was stirred at room temperature for 14 hours, and then quenched with 0.100 ml dried methanol. The solvent was removed under vacuum and the residue was coevaporated 3 times each with 1.00 ml of dried DMF. The activated 5′-AMP was dissolved in dried DMF (1 mL) and compound 7ff (37 mg, 0.10 mmol, 1.0 eq) was added. After stirring at room temperature for 4 days, H₂O was added to quench the reaction at 0° C. The resulting mixture was continued stirring at room temperature for 24 hours. The reaction was then concentrated in vacuo and the crude product was purified via preparative HPLC. Fractions containing the desired product were concentrated and lyophilized to yield the corresponding NAD⁺23.

NAD⁺22 is prepared following the procedure as described above with the necessary modifications well-understood by the skilled artisan.

### Example 7. Synthesis of NAD⁺24-25

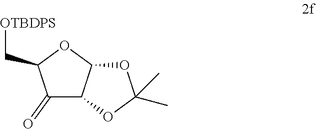

General procedure for the synthesis of compound 2f: To a stirred solution of (3 aR, 5R,6 S,6aR)-5-(hydroxymethyl)-2,2-dimethyltetrahydrofuro[2,3-d][1,3]dioxol-6-ol (prepared according to the reported method in Nucleosides, Nucleotides and Nucleic Acids, 2013, 32, 646-659)(1.0 eq) in DMF (24 mL) was added the Imidazole (2 eq) and TBDPSCl (1.1 eq) at 0° C. The reaction mixture was allowed to warm to room temperature. After stirring at this temperature for 24 hours, the reaction mixture was diluted with EtOAc (100 mL), and the organic phase was washed with water (5×50 mL), dried over anhydrous Na₂SO₄, filtered and concentrated to give a residue. The residue was purified by a flash column chromatography on silica gel to afford the desired product. The product was oxidized by Swern-Oxidation according to the standard procedure to offer the compound 2f.

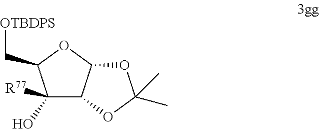

General procedure for the synthesis of compound 3gg: To a stirred solution of compound 2f (1.0 eq) in THF (24 mL) was added the corresponding R⁷⁷MgBr (3.0 eq) at 0° C. If the target compound is, for example NAD⁺24 then R⁷⁷ is —C≡CH. If the target compound is, for example NAD⁺24 then R⁷⁷ is —CH₂C ≡CH. The reaction mixture was allowed to warm to room temperature. After stirring at this temperature for 24 hours, the reaction mixture was diluted with EtOAc (100 mL), and the organic phase was washed with water (5×50 mL), dried over anhydrous Na₂SO₄, filtered and concentrated to give a residue. The residue was purified by a flash column chromatography on silica gel to afford the corresponding compound 3gg.

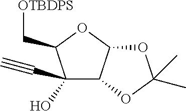

(3aR,5R,6R,6aR)-5-(((tert-butyldiphenylsilyl)oxy)methyl)-6-ethynyl-2,2-dimethyltetrahydrofuro[2,3-d][1,3]dioxol-6-ol. ¹H NMR (400 MHz, CDCl₃): δ 1.07 (s, 9H, 3CH₃), 1.37 (s, 3H, CH₃), 1.59 (s, 3H, CH₃), 2.51 (s, 1H, CH), 3.97-4.02 (m, 2H, CH+CH₂), 4.07 (dd, 1H, J=12.0, 6.0 Hz, CH₂), 4.58 (d, 1H, J=3.6 Hz, CH), 5.85 (d, 1H, J=3.6 Hz, CH), 7.36-7.45 (m, 6H, ArH), 7.67-7.72 (m, 4H, ArH).

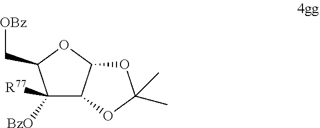

General procedure for the synthesis of compound 4gg: To a 0° C. solution of compound 3gg (2.1 mmol) in anhydrous THF (25 mL) was added AcOH (180 μL, 3.2 mmol, 1.5 eq) followed by the addition of TBAF (3.2 mL, 3.2 mmol, 1.0 M in THF, 1.5 eq). Then reaction mixture was allowed to warm to room temperature. After stirring at this temperature for 14 hours, the reaction mixture was concentrated under reduced pressure to give a residue. The residue was dissolved in a mixture of anhydrous DCM (10 mL) and anhydrous pyridine (10 mL) followed by the addition of BzCl (588 μL, 5.1 mmol, 3 eq) at 0° C. Then the reaction mixture was allowed to warm to room temperature. After stirring for 24 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the corresponding compound 4gg.

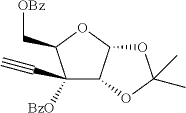

((3aR,5R,6R,6aR)-6-(benzoyloxy)-6-ethynyl-2,2-dimethyltetrahydrofuro[2,3-d][1,3]dioxol-5-yl)methyl benzoate. ¹H NMR (400 MHz, CDCl₃): δ 1.34 (s, 3H, CH₃), 1.51 (s, 3H, CH₃), 2.74 (s, 1H, CH), 4.66 (dd, 1H, J=6.8, 5.2 Hz, CH), 4.73 (dd, 1H, J=11.6, 6.8 Hz, CH₂), 4.82 (dd, 1H, J=11.6, 5.2 Hz, CH₂), 5.32 (d, 1H, J=3.6 Hz, CH), 6.02 (d, 1H, J=3.6 Hz, CH), 7.38-7.42 (m, 4H, ArH), 7.54-7.59 (m, 2H, ArH), 8.02 (dd, 2H, J=8.0, 1.2 Hz, ArH), 8.10 (dd, 2H, J=8.4, 1.2 Hz, ArH).

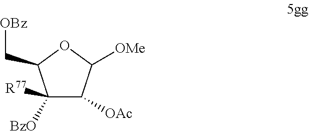

General procedure for the synthesis of compound 5gg: To a solution of compound 4gg (1.7 mmol) in a mixture of anhydrous MeOH was added AcCl (2.0 eq) at r.t. to offer an intermediate compound A. Then the compound A was dissolved in anhydrous pyridine (10 mL) followed by the addition of AcCl (5.1 mmol, 3 eq) at 0° C. Then the reaction mixture was allowed to warm to room temperature. After stirring for 24 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the the corresponding compound 5gg.

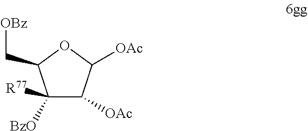

General procedure for the synthesis of compound 6gg: Compound 5gg (1.3 mmol) was dissolved in a mixture of TFA/H₂O (9/1, 15 mL) and the resulting mixture was stirred at room temperature until the reaction complete (monitoring by TLC). Then the reaction was diluted with DCM (60 mL) and the solution was added dropwise to a stirred mixture of ice and saturated aqueous NaHCO₃. Solid NaHCO₃ was added during the addition to maintain a PH of 7. The mixture was extracted with DCM (3×50 mL), and the combined organic extracts was washed with H₂O (50 mL), brine (50 mL), dried over anhydrous Na₂SO₄, filtered, concentrated to give a residue. The residue was dissolved in pyridine (15 mL) and cooled to 0° C. Ac₂O (0.5 mL) was added dropwise and then the resulting mixture was allowed to warm to room temperature. After stirring for 6 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the corresponding compound 6gg.

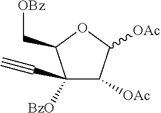

(3R,4R,5R)-4-(benzoyloxy)-5-((benzoyloxy)methyl)-4-ethynyltetrahydrofuran-2,3-diyl diacetate. ¹H NMR (400 MHz, CDCl₃): δ 2.04 (s, 4.5H, CH₃), 2.08 (s, 3H, CH₃), 2.09 (s, 4.5H, CH₃), 2.10 (s, 3H, CH₃), 2.76 (s, 1H, CH), 2.83 (s, 1.5H, CH), 4.72-4.78 (m, 3H, CH+CH₂), 4.82-4.96 (m, 4.5H, CH+CH₂), 5.89 (d, 1H, J=4.4 Hz, CH), 5.98 (d, 1.5H, J=1.2 Hz, CH), 6.21 (d, 1.5H, J=1.2 Hz, CH), 6.60 (d, 1H, J=4.4 Hz, CH), 7.41-7.47 (m, 10.0H, ArH), 7.55-7.62 (m, 5.0H, ArH), 8.00-8.13 (m, 10.0H, ArH).

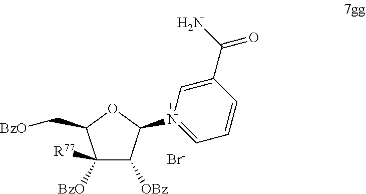

General procedure for the synthesis of compound 7gg: Compound 6gg (0.70 mmol) was dissolved in toluene (10 mL) and cooled to 0° C. HBr (33 wt % in acetic acid) (257 mg, 1.05 mmol, 1.5 eq) was added dropwise and the reaction was stirred at 0° C. for 5 hours. After the starting material was consumed, the reaction was concentrated under reduced pressure to give a residue. The residue was azeotroped with toluene (3×20 mL) to remove remaining acetic acid and dried in vacuo. The crude product and nicotinamide (103 mg, 0.84 mmol, 1.2 eq) was dissolved in CH₃CN (20 mL). The reaction was stirred under Ar gas at room temperature for 24 hours. The reaction was concentrated in vacuo (the temperature was kept below 35° C.) and purified by a flash column chromatography on silica gel to afford the corresponding compound 7gg.

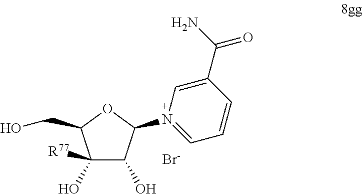

General procedure for the synthesis of compound 8gg: Compound 7gg (0.45 mmol) was dissolved in ammonia (18 mL, 7 N in MeOH) and the reaction was stirred at −10° C. for 48 hours. The reaction was concentrated under reduced pressure and the crude product was dissolved in MeOH (0.5 mL). Addition of ethyl ether (10 mL) resulted in ppt of the desired product. The procedure was repeated three times to yield the corresponding desired product 8gg.

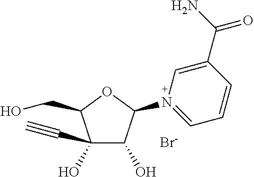

3-carbamoyl-1-((2R,3R,4S,5R)-4-ethynyl-3,4-dihydroxy-5-(hydroxymethyl)tetrahydrofuran-2-yl)pyridin-1-ium bromide. MS (ESI) (ESI) Calcd. For C₁₃H₁₄N₂O₅⁺¹ (M)⁺ requires 279.1, Found: 280.7.

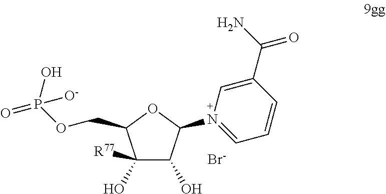

General procedure for the synthesis of compound 9gg: To a stirred solution of compound 8gg (0.27 mmol) in trimethylphosphate (2 mL) was added P(O)Cl₃ (175 μL, 1.89 mmol, 7 eq) at 0° C. and the resulting mixture was stirred at 0° C. for 6 hours. A few drops H₂O was then added to quench the reaction. Trimethylphosphate was removed by extraction with ethyl ether (3×20 ml). The remaining trimethylphosphate was removed by a second extraction with THF (5 ml). The aqueous layer was concentrated in vacuo and the crude product was dissolved in MeOH (0.5 mL). Addition of ethyl ether (10 mL) resulted in ppt of the desired product. The procedure was repeated three times to yield the corresponding desired products 9gg.

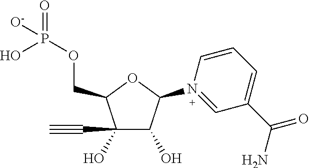

((2R,3S,4R,5R)-5-(3-carbamoylpyridin-1-ium-1-yl)-3-ethynyl-3,4-dihydroxytetrahydrofuran-2-yl)methyl hydrogen phosphate. MS (ESI) Calcd. For C₁₃H₁₆N₂O₈P⁺¹ (M)⁺ requires 359.1, Found: 360.5.

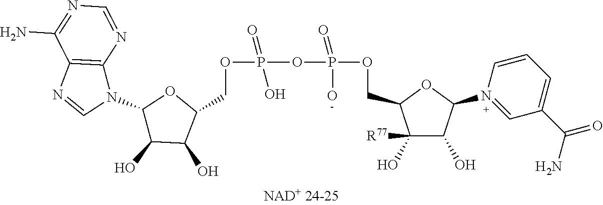

General procedure for the synthesis of NAD⁺24: To a stirred solution of Adenosine 5′-monophosphate (5′-AMP) (52 mg, 0.15 mmol, 1.5 eq) in dried DMF (2 mL) were added 1,1-carbonyldiimidazole (CDI) (63 mg, 0.50 mmol, 5 eq) and triethylamine (23 μL, 0.16 mmol. 1.6 eq). The reaction mixture was stirred at room temperature for 14 hours, and then quenched with 0.100 ml dried methanol. The solvent was removed under vacuum and the residue was coevaporated 3 times each with 1.00 ml of dried DMF. The activated 5′-AMP was dissolved in dried DMF (1 mL) and compound 9gg (37 mg, 0.10 mmol, 1.0 eq) was added. After stirring at room temperature for 4 days, H₂O was added to quench the reaction at 0° C. The resulting mixture was continued stirring at room temperature for 24 hours. The reaction was then concentrated in vacuo and the crude product was purified via preparative HPLC. Fractions containing the desired product were concentrated and lyophilized to yield the corresponding NAD⁺24-25.

NAD⁺25 is prepared following the procedure as described above with the necessary modifications well-understood by the skilled artisan.

### Example 9. Synthesis of NAD⁺27

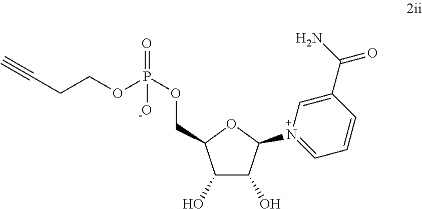

General procedure for the synthesis of compound (2ii): To a stirred solution of 3-carbamoyl-1-((2R,3R,4 S,5R)-3,4-dihydroxy-5-(hydroxymethyl)tetrahydrofuran-2-yl)pyridin-1-ium bromide (0.27 mmol) in trimethylphosphate (2 mL) was added P(O)Cl₃ (175 μL, 1.89 mmol, 7 eq) at 0° C. and the resulting mixture was stirred at 0° C. for 6 hours. But-3-yn-1-ol (14 eq) was then added to quench the reaction. Trimethylphosphate was removed by extraction with ethyl ether (3×20 ml). The remaining trimethylphosphate was removed by a second extraction with THF (5 ml). The aqueous layer was concentrated in vacuo and the crude product was dissolved in MeOH (0.5 mL). Addition of ethyl ether (10 mL) resulted in ppt of the desired product. The procedure was repeated three times to yield the desired product 2ii.

but-3-yn-1-yl (((2R,3S,4R,5R)-5-(3-carbamoylpyridin-1-ium-1-yl)-3,4-dihydroxytetrahydrofuran-2-yl)methyl) phosphate. MS (ESI) Calcd. For C₁₅H₂₀N₂O₈P⁺¹ (M)⁺ requires 387.1, Found: 387.1.

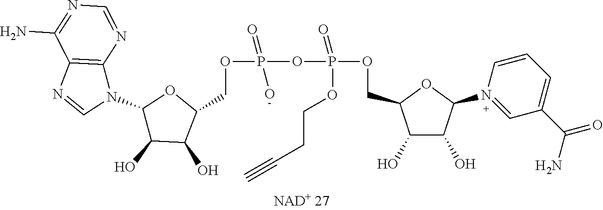

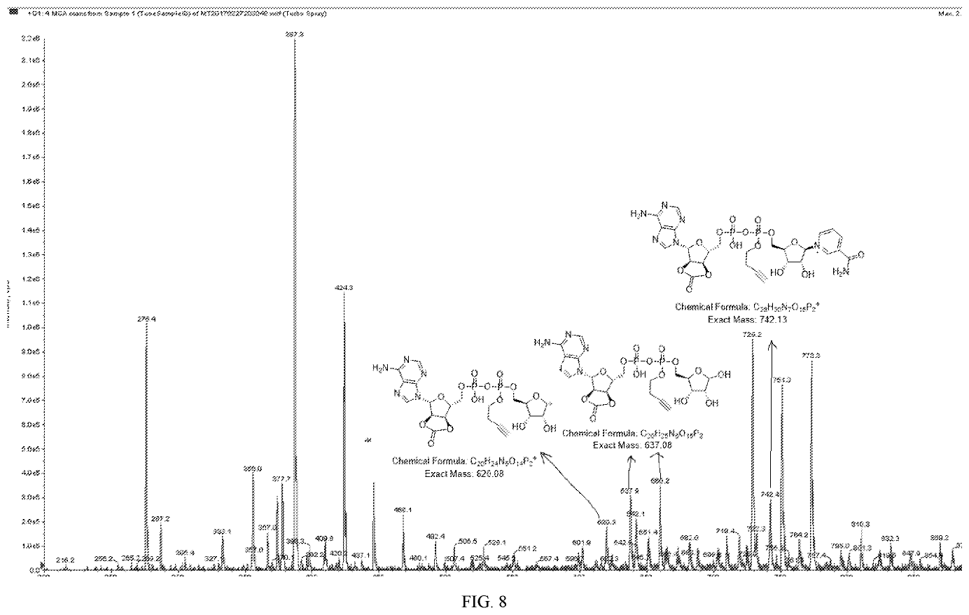

General procedure for the synthesis of NAD⁺27: To a stirred solution of Adenosine 5′-monophosphate (5′-AMP) (52 mg, 0.15 mmol, 1.5 eq) in dried DMF (2 mL) is added 1,1-carbonyldiimidazole (CDI) (63 mg, 0.50 mmol, 5 eq) and triethylamine (23 μL, 0.16 mmol. 1.6 eq). The reaction mixture is stirred at room temperature for 14 hours, and is then quenched with 0.100 ml dried methanol. The solvent is removed under vacuum and the residue is coevaporated 3 times each with 1.00 ml of dried DMF. The activated 5′-AMP is dissolved in dried DMF (1 mL) and compound 2ii (0.10 mmol, 1.0 eq) is added. After stirring at room temperature for 4 days, H₂O is added to quench the reaction at 0° C. The resulting mixture is continued stirring at room temperature for 24 hours. The reaction is then concentrated in vacuo and the crude product is purified via preparative HPLC. Fractions containing the desired product are concentrated and are lyophilized to yield NAD⁺27. FIG. 8 shows the MS (ESI) analysis of the reaction the synthesize NAD⁺27.

### Example 9. Synthesis of NAD⁺28

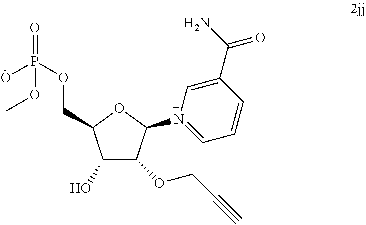

General procedure for the synthesis of compound 2jj: To a stirred solution of 3-carbamoyl-1-((2R,3R,4R,5R)-4-hydroxy-5-(hydroxymethyl)-3-(prop-2-yn-1-yloxy)tetrahydrofuran-2-yl)pyridin-1-ium bromide (0.27 mmol) in trimethylphosphate (2 mL) was added P(O)Cl₃ (175 μL, 1.89 mmol, 7 eq) at 0° C. and the resulting mixture was stirred at 0° C. for 6 hours. MeOH (14 eq) was then added to quench the reaction. Trimethylphosphate was removed by extraction with ethyl ether (3×20 ml). The remaining trimethylphosphate was removed by a second extraction with THF (5 ml). The aqueous layer was concentrated in vacuo and the crude product was dissolved in MeOH (0.5 mL). Addition of ethyl ether (10 mL) resulted in ppt of the desired product. The procedure was repeated three times to yield the desired product 2jj.((2R,3R,4R,5R)-5-(3-carbamoylpyridin-1-ium-1-yl)-3-hydroxy-4-(prop-2-yn-1-yloxy)tetrahydrofuran-2-yl)methyl methyl phosphate. MS (ESI) Calcd. For C₁₅H₂₀N₂O₈P⁺¹ (M)⁺ requires 387.1, Found: 387.8.

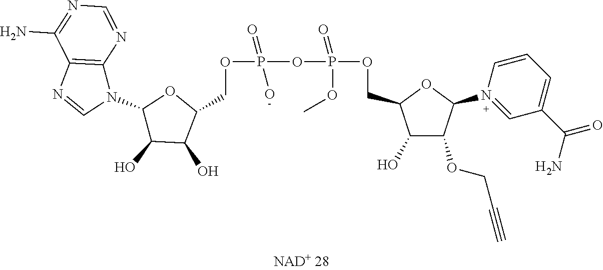

General procedure for the synthesis of NAD⁺28: To a stirred solution of Adenosine 5′-monophosphate (5′-AMP) (52 mg, 0.15 mmol, 1.5 eq) in dried DMF (2 mL) is added 1,1-carbonyldiimidazole (CDI) (63 mg, 0.50 mmol, 5 eq) and triethylamine (23 μL, 0.16 mmol. 1.6 eq). The reaction mixture is stirred at room temperature for 14 hours, and is then quenched with 0.100 ml dried methanol. The solvent is removed under vacuum and the residue is coevaporated 3 times each with 1.00 ml of dried DMF. The activated 5′-AMP is dissolved in dried DMF (1 mL) and compound 2jj (0.10 mmol, 1.0 eq) was added. After stirring at room temperature for 4 days, H₂O is added to quench the reaction at 0° C. The resulting mixture is continued stirring at room temperature for 24 hours. The reaction is then concentrated in vacuo and the crude product is purified via preparative HPLC (C18-A column, 150×4.6 mm, 5 μm) (mobile phase A: 0.1% formic acid (aq), mobile B: 0.1% formic acid in acetonitrile; flow rate=1.0 ml/min; 0-16 min: 0-6.7% B, 16-18 min: 6.7-0% B). Fractions containing the desired product are concentrated and are lyophilized to yield NAD⁺28.

### Example 10. Synthesis of NAD⁺29

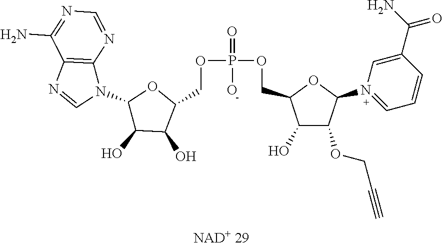

General procedure for the synthesis of NAD⁺29: To a stirred solution of 3-carbamoyl-1-((2R,3R,4R,5R)-4-hydroxy-5-(hydroxymethyl)-3-(prop-2-yn-1-yloxy)tetrahydrofuran-2-yl)pyridin-1-ium bromide (0.27 mmol) in trimethylphosphate (2 mL) is added P(O)Cl₃ (175 μL, 1.89 mmol, 7 eq) at 0° C. and the resulting mixture is stirred at 0° C. for 6 hours. Adenine (30 eq) is then added to quench the reaction. The reaction is then concentrated in vacuo and the crude product is purified via preparative HPLC (C18-A column, 150×4.6 mm, 5 μm) (mobile phase A: 0.1% formic acid (aq), mobile B: 0.1% formic acid in acetonitrile; flow rate=1.0 ml/min; 0-16 min: 0-6.7% B, 16-18 min: 6.7-0% B). Fractions containing the desired product are concentrated and are lyophilized to yield NAD⁺29.

### Example 11. Synthesis of NAD⁺30

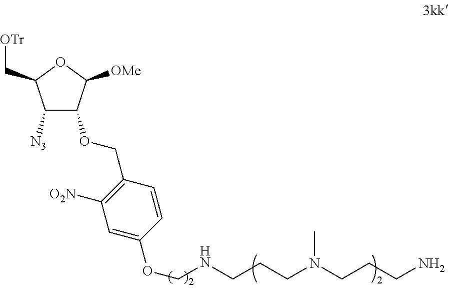

General procedure for the synthesis of compound 3kk′: To a stirred solution of compound 2kk (prepared as described below, 3.7 mmol) in anhydrous DMF (25 mL) was added NaH (180 mg, 4.5 mmol, 1.2 eq, 60% dispersion in mineral oil) at 0° C. followed by the addition of N1-(3-aminopropyl)-N4-(3-((2-(4-(bromomethyl)-3-nitrophenoxy)ethyl)amino)propyl)-N1,N4-dimethylbutane-1,4-diamine (5.6 mmol, 1.5 eq) at the same temperature. Then reaction mixture was allowed to warm to room temperature. After stirring at this temperature for 6 hours, the reaction was then concentrated in vacuo and the crude product was purified via preparative HPLC (C18-A column, 150×4.6 mm, 5 μm) (mobile phase A: 0.1% formic acid (aq), mobile B: 0.1% formic acid in acetonitrile; flow rate=1.0 ml/min; 0-16 min: 0-6.7% B, 16-18 min: 6.7-0% B). Fractions containing the desired product were concentrated and lyophilized to yield the compound 3kk′.

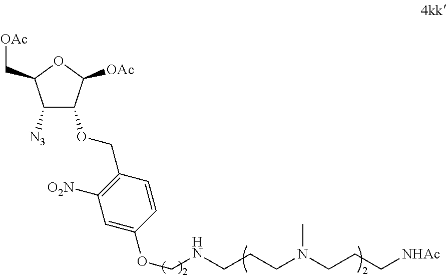

General procedure for the synthesis of (2R,3R,4R,5S)-5-acetoxy-2-((benzoyloxy)methyl)-4-(prop-2-yn-1-yloxy)tetrahyd-rofuran-3-yl benzoate (4kk′): Compound 3kk′ (534 mg, 1.3 mmol) was dissolved in a mixture of TFA/H₂O (9/1, 15 mL) and the resulting mixture was stirred at room temperature until the reaction complete (monitoring by TLC). Then the reaction was concentrated to give a residue. The residue was dissolved in pyridine (15 mL) and cooled to 0° C. Ac₂O (0.5 mL) was added dropwise and then the resulting mixture was allowed to warm to room temperature. After stirring for 6 hours, the reaction was quenched with MeOH (10 mL) and the mixture was concentrated under reduced pressure to give a residue. The residue was was purified via preparative HPLC (C18-A column, 150×4.6 mm, 5-m) (mobile phase A: 0.1% formic acid (aq), mobile B: 0.1% formic acid in acetonitrile; flow rate=1.0 ml/min; 0-16 min: 0-6.7% B, 16-18 min: 6.7-0% B). Fractions containing the desired product were concentrated and lyophilized to yield the compound 4kk′.

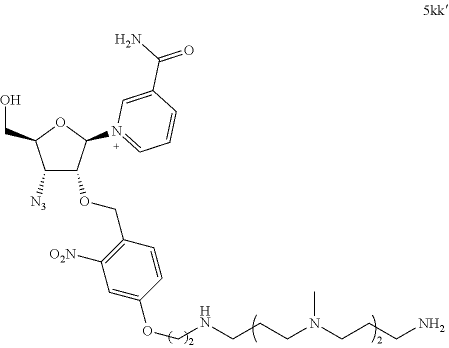

General procedure for the synthesis of 1-((2R,3R,4R,5R)-4-(benzoyloxy)-5-((benzoyloxy)methyl)-3-(prop-2-yn-1-yloxy)t-etrahydrofuran-2-yl)-3-carbamoyl-pyridin-1-ium bromide (5kk′): Compound 4kk′ (307 mg, 0.70 mmol) was dissolved in toluene (10 mL) and cooled to 0° C. HBr (33 wt % in acetic acid) (257 mg, 1.05 mmol, 1.5 eq) was added dropwise and the reaction was stirred at 0° C. for 5 hours. After the starting material was consumed, the reaction was concentrated under reduced pressure to give a residue. The residue was azeotroped with toluene (3×20 mL) to remove remaining acetic acid and dried in vacuo. The crude product and nicotinamide (103 mg, 0.84 mmol, 1.2 eq) was dissolved in CH₃CN (20 mL). The reaction was stirred under Ar gas at room temperature for 24 hours. The reaction was concentrated in vacuo (the temperature was kept below 35° C.) to give a residue. The residue was dissolved in ammonia (18 mL, 7 N in MeOH) and the reaction was stirred at −10° C. for 48 hours. The reaction was concentrated under reduced pressure and the crude product was dissolved in MeOH (0.5 mL). Addition of ethyl ether (10 mL) resulted in ppt of the desired product. The procedure was repeated three times to yield the desired product 5kk′.

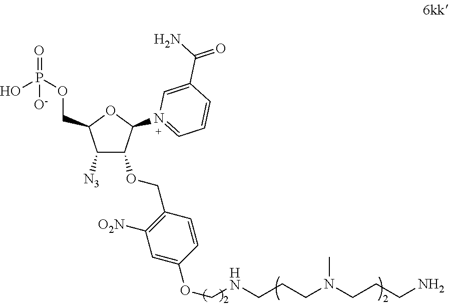

General procedure for the synthesis of compound 6kk′: To a stirred solution of compound 5kk′ (0.27 mmol) in trimethylphosphate (2 mL) was added P(O)Cl₃ (175 μL, 1.89 mmol, 7 eq) at 0° C. and the resulting mixture was stirred at 0° C. for 6 hours. A few drops H₂O was then added to quench the reaction. Trimethylphosphate was removed by extraction with ethyl ether (3×20 ml). The remaining trimethylphosphate was removed by a second extraction with THF (5 ml). The aqueous layer was concentrated in vacuo and the crude product was dissolved in MeOH (0.5 mL). Addition of ethyl ether (10 mL) resulted in ppt of the desired product. The procedure was repeated three times to yield the desired product 6kk′.

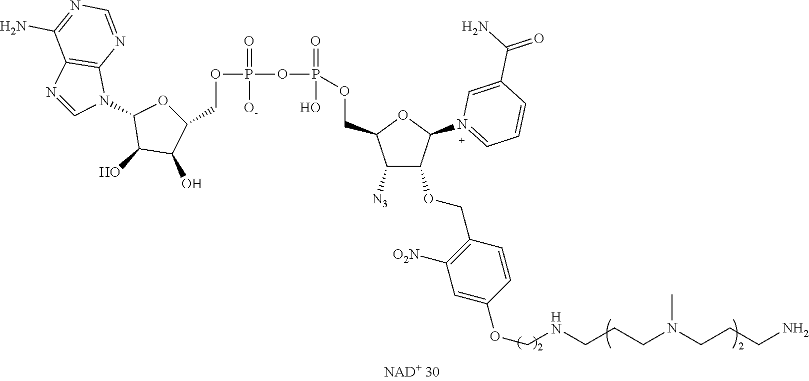

General procedure for the synthesis of NAD⁺30: To a stirred solution of Adenosine 5′-monophosphate (5′-AMP) (52 mg, 0.15 mmol, 1.5 eq) in dried DMF (2 mL) is added 1,1-carbonyldiimidazole (CDI) (63 mg, 0.50 mmol, 5 eq) and triethylamine (23 μL, 0.16 mmol. 1.6 eq). The reaction mixture is stirred at room temperature for 14 hours, and is then quenched with 0.100 ml dried methanol. The solvent is removed under vacuum and the residue is coevaporated 3 times each with 1.00 ml of dried DMF. The activated 5′-AMP is dissolved in dried DMF (1 mL) and compound 6kk′ (37 mg, 0.10 mmol, 1.0 eq) is added. After stirring at room temperature for 4 days, H₂O is added to quench the reaction at 0° C. The resulting mixture is continued stirring at room temperature for 24 hours. The reaction is then concentrated in vacuo and the crude product is purified via preparative HPLC (C18-A column, 150×4.6 mm, 5 μm) (mobile phase A: 0.1% formic acid (aq), mobile B: 0.1% formic acid in acetonitrile; flow rate=1.0 ml/min; 0-16 min: 0-6.7% B, 16-18 min: 6.7-0% B). Fractions containing the desired product are concentrated and are lyophilized to yield NAD⁺30.

### Example 12. Synthesis of NAD⁺31

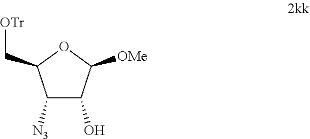

General procedure for the synthesis of compound 2kk: To a solution of (2R,3R,4S,5S)-4-azido-5-(hydroxymethyl)-2-methoxytetrahydrofuran-3-ol (1.7 mmol) in anhydrous DMF (10 mL) were added DAMP (0.1 eq), EtN₃ (3 eq) and TrCl (2 eq) at 0° C. Then the reaction mixture was allowed to warm to room temperature. After stirring for 24 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the compound 2kk.

(2R,3R,4S,5S)-4-azido-2-methoxy-5-((trityloxy)methyl)tetrahydrofuran-3-ol. ¹H NMR (400 MHz, CDCl₃): δ 2.18 (d, 1H, J=2.4 Hz, OH), 3.21 (dd, 1H, J=9.6, 4.0 Hz, CH₂), 3.37-3.40 (m, 4H, CH+CH₂), 4.15-4.22 (m, 3H, 3CH), 4.87 (s, 1H, CH), 7.22-7.26 (m, 3H, ArH), 7.29-7.33 (m, 6H, ArH), 7.48-7.50 (m, 6H, ArH).

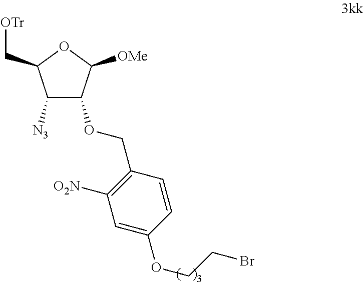

General procedure for the synthesis of compound 3kk: To a stirred solution of compound 2kk (3.7 mmol) in anhydrous DCM (25 mL) was added Bu₄NI (1.85 mmol, 0.5 eq) and NaOH (8 mL, 40% aq) at 0° C. followed by the addition of 4-(4-bromobutoxy)-2-(bromomethyl)-1-nitrobenzene (5.6 mmol, 1.5 eq) at the same temperature. Then reaction mixture was allowed to warm to room temperature. After stirring at this temperature for 6 hours, the reaction was then concentrated in vacuo and to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the corresponding compound 3kk.

(2S,3R,4R,5R)-3-azido-4-((4-(4-bromobutoxy)-2-nitrobenzyl)oxy)-5-methoxy-2-((trityloxy)methyl)tetrahydrofuran. ¹H NMR (400 MHz, CDCl₃): δ 1.96-2.10 (m, 4H, 2CH₂), 3.24 (dd, 1H, J=10.4, 4.0 Hz, CH₂), 3.38-3.42 (m, 4H, CH+CH₃), 3.47 (t, 2H, J=6.4 Hz, CH₂), 4.02 (dd, 1H, J=8.0, 4.8 Hz, CH), 4.08-4.13 (m, 3H, CH+CH₂), 4.33-4.37 (m, 1H, CH), 5.04 (s, 1H, CH), 5.09 (d, 1H, J=15.6 Hz, CH₂), 5.16 (d, 1H, J=15.6 Hz, CH₂), 6.87 (dd, 1H, J=9.2, 2.8 Hz, ArH), 7.23-7.26 (m, 3H, ArH), 7.30-7.33 (m, 6H, ArH), 7.37 (d, 1H, J=2.8 Hz, ArH), 7.48-7.51 (m, 6H, ArH), 8.19 (d, 1H, J=9.2 Hz, ArH).

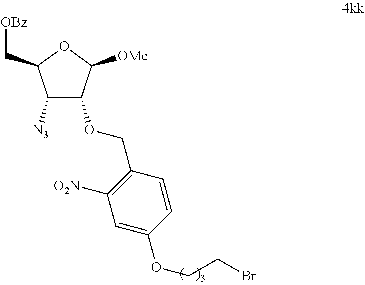

General procedure for the synthesis of compound (4kk): To a stirred solution of compound 3kk (2.3 mmol) in a MeOH (20 mL) was added cocn.HCl (2 mL) at 0° C. Then the resulting mixture was stirred at room temperature until the reaction complete (monitoring by TLC). Then the reaction was concentrated to give a residue. The residue was dissolved in pyridine (15 mL) and cooled to 0° C. BzCl (3.5 mmol, 1.5 eq) was added dropwise and then the resulting mixture was allowed to warm to room temperature. After stirring for 6 hours, the reaction was quenched with MeOH (10 mL) and the mixture was concentrated under reduced pressure to give a residue. The residue was purified by a flash column chromatography on silica gel to afford the corresponding compound 4kk.

((2S,3R,4R,5R)-3-azido-4-((4-(4-bromobutoxy)-2-nitrobenzyl)oxy)-5-methoxytetrahydrofuran-2-yl)methyl benzoate. ¹H NMR (400 MHz, CDCl₃): δ 1.97-2.11 (m, 4H, 2CH₂), 3.35 (s, 3H, CH₃), 3.48 (t, 2H, J=6.4 Hz, CH₂), 4.03 (dd, 1H, J=7.2, 4.4 Hz, CH), 4.10-4.16 (m, 3H, CH₂+CH), 4.46 (dd, 1H, J=11.6, 4.8 Hz, CH₂), 4.54-4.58 (m, 1H, CH), 4.63 (dd, 1H, J=11.6, 4.4 Hz, CH), 5.04 (s, 1H, CH), 5.12 (d, 1H, J=15.6 Hz, CH₂), 5.19 (d, 1H, J=15.6 Hz, CH₂), 6.88 (dd, 1H, J=9.2, 2.8 Hz, ArH), 7.37 (d, 1H, J=2.8 Hz, ArH), 7.46 (t, 2H, J=7.6 Hz, ArH), 7.57-7.61 (m, 1H, ArH), 8.08-8.10 (m, 2H, ArH), 8.19 (d, 1H, J=9.2 Hz, ArH).

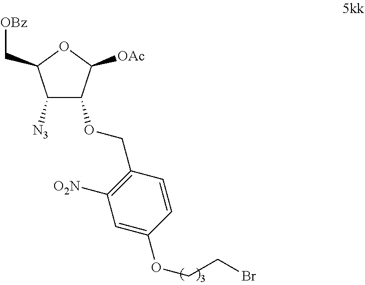

General procedure for the synthesis of compound 5kk: Compound 4kk (1.3 mmol) was dissolved in a mixture of TFA/H₂O (9/1, 15 mL) and the resulting mixture was stirred at room temperature until the reaction complete (monitoring by TLC). Then the reaction was diluted with DCM (60 mL) and the solution was added dropwise to a stirred mixture of ice and saturated aqueous NaHCO₃. Solid NaHCO₃ was added during the addition to maintain a PH of 7. The mixture was extracted with DCM (3×50 mL), and the combined organic extracts was washed with H₂O (50 mL), brine (50 mL), dried over anhydrous Na₂SO₄, filtered, concentrated to give a residue. The residue was dissolved in pyridine (15 mL) and cooled to 0° C. Ac₂O (0.5 mL) was added dropwise and then the resulting mixture was allowed to warm to room temperature. After stirring for 6 hours, the reaction was quenched with MeOH (10 mL) and the mixture was concentrated under reduced pressure to give a residue. The residue was dissolved in EtOAc (50 mL), and the organic phase was washed successively with saturated aqueous CuSO₄ (3×50 mL), brine (50 mL), dried over anhydrous Na₂SO₄, filtered, concentrated and purified by a flash column chromatography on silica gel to afford the compound 5kk.

((2S,3R,4R)-5-acetoxy-3-azido-4-((4-(4-bromobutoxy)-2-nitrobenzyl)oxy)tetrahydrofuran-2-yl)methyl benzoate. ¹H NMR (400 MHz, CDCl₃): δ 1.93 (s, 3H, CH₃), 1.98-2.10 (m, 4H, 2CH₂), 3.49 (t, 2H, J=6.4 Hz, CH₂), 4.04 (dd, 1H, J=8.4, 4.8 Hz, CH), 4.12 (t, 2H, J=6.0 Hz, CH₂), 4.25 (d, 1H, J=4.8 Hz, CH), 4.49 (dd, 1H, J=12.0, 4.4 Hz, CH₂), 4.59-4.63 (m, 1H, CH), 4.72 (dd, 1H, J=12.0, 4.0 Hz, CH), 5.20 (s, 2H, CH₂), 6.28 (s, 1H, CH), 6.89 (dd, 1H, J=9.2, 2.8 Hz, ArH), 7.32 (d, 1H, J=2.8 Hz, ArH), 7.46 (t, 2H, J=8.0 Hz, ArH), 7.57-7.62 (m, 1H, ArH), 8.09 (dd, 2H, J=8.0, 1.2 Hz, ArH), 8.20 (d, 1H, J=9.2 Hz, ArH).

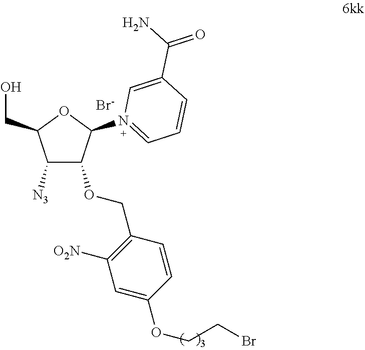

General procedure for the synthesis of compound (6kk): Compound 5kk (0.70 mmol) was dissolved in toluene (10 mL) and cooled to 0° C. HBr (33 wt % in acetic acid) (257 mg, 1.05 mmol, 1.5 eq) was added dropwise and the reaction was stirred at 0° C. for 5 hours. After the starting material was consumed, the reaction was concentrated under reduced pressure to give a residue. The residue was azeotroped with toluene (3×20 mL) to remove remaining acetic acid and dried in vacuo. The crude product and nicotinamide (103 mg, 0.84 mmol, 1.2 eq) was dissolved in CH₃CN (20 mL). The reaction was stirred under Ar gas at room temperature for 24 hours. The reaction was concentrated in vacuo (the temperature was kept below 35° C.) to give a residue. The residue was dissolved in ammonia (18 mL, 7 N in MeOH) and the reaction was stirred at −10° C. for 48 hours. The reaction was concentrated under reduced pressure and the crude product was dissolved in MeOH (0.5 mL). Addition of ethyl ether (10 mL) resulted in ppt of the desired product. The procedure was repeated three times to yield the desired product 6kk.

1-((2R,3R,4R,5 S)-4-azido-3-((4-(4-bromobutoxy)-2-nitrobenzyl)oxy)-5-(hydroxymethyl)tetrahydrofuran-2-yl)-3-carbamoylpyridin-1-ium bromide. ¹H NMR (400 MHz, CD₃OD): δ 1.94-2.07 (m, 4H, 2CH₂), 3.55 (t, 2H, J=6.4 Hz, CH₂), 3.80 (dd, 1H, J=12.4, 2.4 Hz, CH₂), 3.88 (dd, 1H, J=12.4, 2.4 Hz, CH₂), 4.05 (t, 1H, J=5.2 Hz, CH), 4.73 (dd, 1H, J=5.2, 1.6 Hz, CH), 4.82-4.83 (m, 1H, CH), 5.18 (d, 1H, J=13.6 Hz, CH₂), 5.23-5.28 (m, 2H, CH₂+CH), 6.72-6.75 (m, 2H, CH+ArH), 7.01 (dd, 1H, J=9.2, 2.8 Hz, ArH), 8.15 (d, 1H, J=9.2 Hz, ArH), 8.22-8.25 (m, 1H, ArH), 9.06 (d, 1H, J=7.6 Hz, ArH), 9.21 (d, 1H, J=6.0 Hz, ArH), 9.41 (s, 1H, ArH).

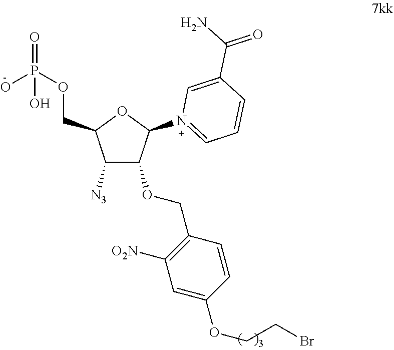

General procedure for the synthesis of compound 7kk: To a stirred solution of compound 6kk (0.27 mmol) in trimethylphosphate (2 mL) was added P(O)Cl₃ (175 μL, 1.89 mmol, 7 eq) at 0° C. and the resulting mixture was stirred at 0° C. for 6 hours. A few drops H₂O was then added to quench the reaction. Trimethylphosphate was removed by extraction with ethyl ether (3×20 ml). The remaining trimethylphosphate was removed by a second extraction with THF (5 ml). The aqueous layer was concentrated in vacuo and the crude product was dissolved in MeOH (0.5 mL). Addition of ethyl ether (10 mL) resulted in ppt of the desired product. The procedure was repeated three times to yield the desired product 7kk.

((2S,3R,4R,5R)-3-azido-4-((4-(4-bromobutoxy)-2-nitrobenzyl)oxy)-5-(3-carbamoylpyridin-1-ium-1-yl)tetrahydrofuran-2-yl)methyl hydrogen phosphate. ¹H NMR (400 MHz, CD₃OD): δ 1.92-2.07 (m, 4H, 2CH₂), 3.54 (t, 2H, J=6.4 Hz, CH₂), 3.99-4.18 (m, 4H, 2CH₂), 4.85-4.92 (2H, 2CH, overlap with the solvent residue peak), 5.17 (d, 1H, J=13.6 Hz, CH₂), 5.32 (d, 1H, J=13.6 Hz, CH₂), 5.42 (br, 1H, CH), 6.67 (d, 1H, J=2.4 Hz, ArH), 6.75 (d, 1H, J=3.6 Hz, CH), 6.98 (dd, 1H, J=9.2, 2.4 Hz, ArH), 8.12 (d, 1H, J=9.2 Hz, ArH), 8.21-8.25 (m, 1H, ArH), 9.05 (d, 1H, J=8.0 Hz, ArH), 9.22 (d, 1H, J=6.4 Hz, ArH), 9.45 (s, 1H, ArH).

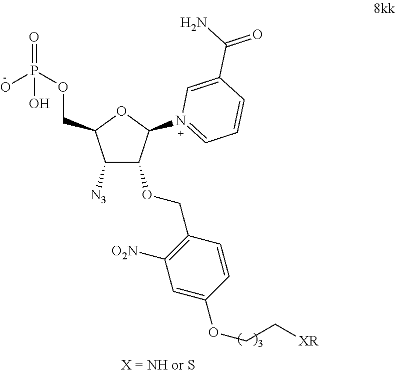

General procedure for the synthesis of compound 8kk: To a stirred solution of compound 7kk (0.2 mmol) in H₂O or MeOH (4 mL) was added RNH₂ or RSK (0.4-1.0 mmol, 2-5 eq) at 0° C. Then the resulting mixture was allowed to warm to room temperature and stirred at the same temperature until the reaction was complete (monitored by HPLC). The reaction was then concentrated in vacuo and the crude product is purified via preparative HPLC. Fractions containing the desired product are concentrated and are lyophilized to yield 8kk.

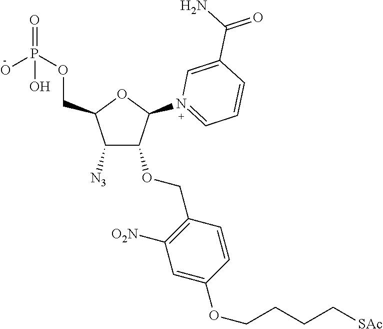

((2S,3R,4R,5R)-4-((4-(4-(acetylthio)butoxy)-2-nitrobenzyl)oxy)-3-azido-5-(3-carbamoylpyridin-1-ium-1-yl)tetrahydrofuran-2-yl)methyl hydrogen phosphate. ¹H NMR (400 MHz, CD₃OD): δ 1.71-1.78 (m, 2H, CH₂), 1.83-1.90 (m, 2H, CH₂), 2.34 (s, 3H, CH₃), 2.96 (t, 2H, J=7.2 Hz, CH₂), 4.96-4.04 (m, 2H, CH₂), 4.06-4.10 (m, 1H, CH₂), 4.13-4.18 (m, 1H, CH₂), 4.85 (d, 1H, J=4.8 Hz, CH), 4.87-4.94 (1H, CH, overlap with the water peak), 5.19 (d, 1H, J=13.6 Hz, CH₂), 5.32 (d, 1H, J=13.6 Hz, CH₂), 5.42 (t, 1H, J=5.6 Hz, CH), 6.65 (d, 1H, J=2.8 Hz, ArH), 6.74 (d, 1H, J=6.4 Hz, CH), 6.97 (dd, 1H, J=9.2, 2.8 Hz, ArH), 8.13 (d, 1H, J=9.2 Hz, ArH), 8.24 (dd, 1H, J=7.6, 6.4 Hz, ArH), 9.05-9.08 (m, 1H, ArH), 9.22 (d, 1H, J=6.4 Hz, ArH), 9.44 (s, 1H, ArH).

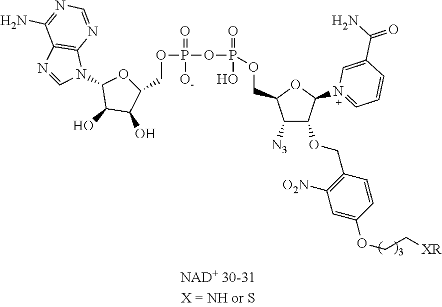

General procedure for the synthesis of NAD⁺31: To a stirred solution of 8kk (0.15 mmol, 1.5 eq) in dried DMF (2 mL) was added 1,1-carbonyldiimidazole (CDI) (63 mg, 0.50 mmol, 5 eq) and triethylamine (23 μL, 0.16 mmol. 1.6 eq). The reaction mixture was stirred at room temperature for 8 hours, and was then quenched with 0.100 ml dried methanol. The solvent was removed under vacuum and the residue was coevaporated 3 times each with 1.00 ml of dried DMF. The activated 8kk was dissolved in dried DMF (1 mL) and compound Adenosine 5′-monophosphate (5′-AMP) (0.10 mmol, 1.0 eq) was added. After stirring at room temperature for 4 days, H₂O was added to quench the reaction at 0° C. The resulting mixture was continued stirring at room temperature for 24 hours. The reaction was then concentrated in vacuo and the crude product was purified via preparative HPLC (C18-A column, 150×4.6 mm, 5 m) Fractions containing the desired product were concentrated and were lyophilized to yield corresponding NAD⁺30 (X═NH) and intermediate A (X═S). The intermediate A was then dissolved in MeOH (2 mL) and DIPEA (0.2 mmol, 2.0 eq) and N-(3-((4-((4-((3-aminopropyl)amino)butyl)amino)butyl)amino)propyl)-3-(2,5-dioxo-2,5-dihydro-1H-pyrrol-1-yl)propanamide (0.3 mmol. 3 eq) at 0° C. Then the resulting mixture was allowed to warm to room temperature and stirred at the same temperature until the reaction was complete (monitored by HPLC). The reaction was then concentrated in vacuo and the crude product is purified via preparative HPLC. Fractions containing the desired product are concentrated and were lyophilized to yield NAD⁺31.

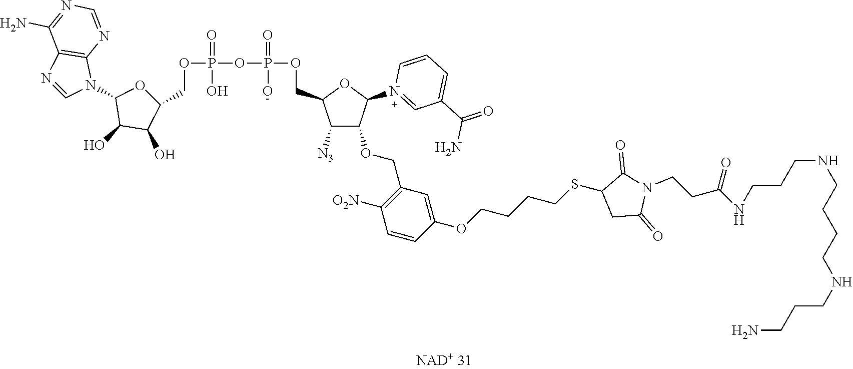

1-((2R,3R,4R,5S)-5-((((((((2R,3 S,4R,5R)-5-(6-amino-9H-purin-9-yl)-3,4-dihydroxytetrahydrofuran-2-yl)methoxy)(hydroxy)phosphoryl)oxy)oxidophosphoryl)oxy)methyl)-3-((5-(4-((1-(3-((3-((4-((3-aminopropyl)amino)butyl)amino)propyl)amino)-3-oxopropyl)-2,5-dioxopyrrolidin-3-yl)thio)butoxy)-2-nitrobenzyl)oxy)-4-azidotetrahydrofuran-2-yl)-3-carbamoylpyridin-1-ium (NAD⁺31). MS (ESI) Calcd. For C₄₉H₇₁N₁₆O₁₉P₂S⁺¹ (M)⁺ requires 1281.4, Found: 1281.6.

### Example 12: Effect of Topotecan on Cellular ADP-Ribosylation

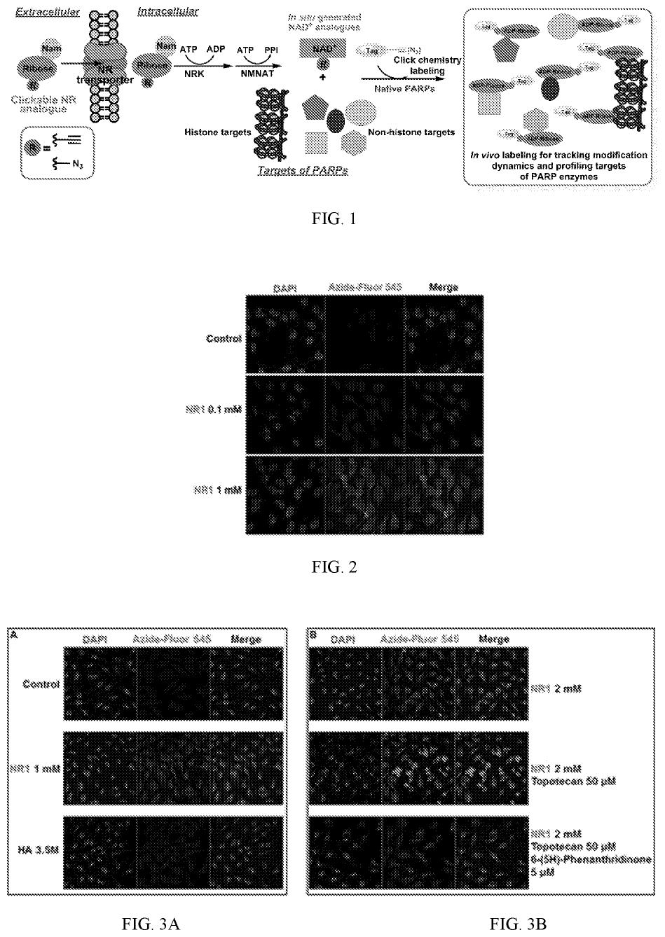

HeLa cells grown in DMEM with 10% FBS were treated with NR¹ at indicated concentrations (0.1 and 1 mM) for 48 hours, followed by fixation, permeabilization, and fluorescent staining through Cu(I)-catalyzed click chemistry using Azide-fluor 545. The stained cells were imaged by confocal microscope (see FIG. 2).

To evaluate the effect of topotecan on cellular ADP-ribosylation, HeLa cells grown in DMEM with 10% FBS were treated with NR1 at indicated concentrations (2 mM) for 6-12 hours in the absence or presence of topotecan and 6-(5H)-phenanthridinone at indicated concentrations (5 um), followed by fixation, permeabilization, and fluorescent staining through Cu(I)-catalyzed click chemistry using Azide-fluor 545. The stained cells were imaged by confocal microscope. To examine the sensitivity of fluorescently labeled ADP-ribosylation to hydroxylamine (HA), cells were first incubated with NR1 (1 mM) for 6 hours, followed by fixation and permeabilization. HA (3.5 M) was used to treat the fixed cells on slides for 60 min. After extensive washing with PBS, the Cu(I)-catalyzed click chemistry was performed using Azide-fluor 545. The stained cells were imaged by confocal microscope (see FIG. 3).

### Example 13: Effect of Topotecan on Cellular ADP-Ribosylation

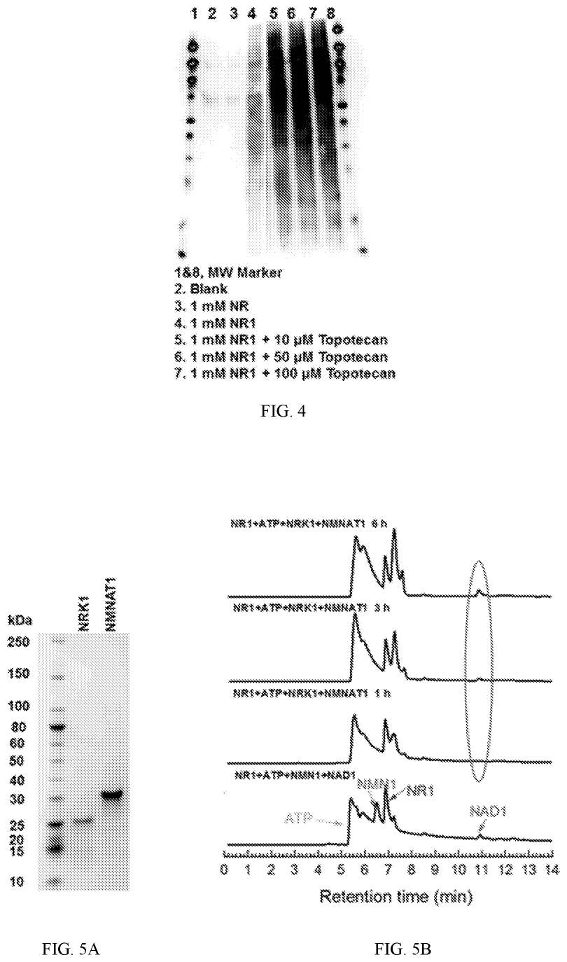

Expi293 cells were treated with NR or NR1 at indicated concentrations in the absence or presence of topotecan (10-100 uM) for 6 hours. The cells were collected by centrifugation, followed by lysis using RIPA buffer. The soluble cell lysates were subjected Cu(I)-catalyzed click chemistry using Azide-biotin. After click reations, cell lysates were loaded onto SDS-PAGE gel for western blot analysis using streptavidin-HRP conjugate for imaging (see FIG. 4).

### Example 14: In Vitro Synthesis of NAD⁺1

Human NRK1 and NMNAT1 were cloned into the pET28(A) bacterial expression vector. The constructs encoding human NRK1 and NMNAT1 that were confirmed by DNA sequencing were then transformed with E. coli BL21 (DE3) cells for overnight expression at 37 degree. Expressed NRK1 and NMNAT1 were purified by Ni-NTA affinity chromatography and analyzed by SDS-PAGE gel (see FIG. 5A). To examine the in vitro biosynthesis of NAD1 from NR1, reactions were set up by adding 1.25 mM ATP, 1 mM NR1, 200 ug/mL purified NRK1 and NMNAT1 into a solution with 50 mM Tris pH 8, 100 mM NaCl, 20 mM MgCl, and 1 mM DTT for incubation of 1-6 hours. The reactions were analyzed by HPLC using C₁₈ reverse phase column (see FIG. 5B).

### Example 15: LC-MS Analysis of NR1

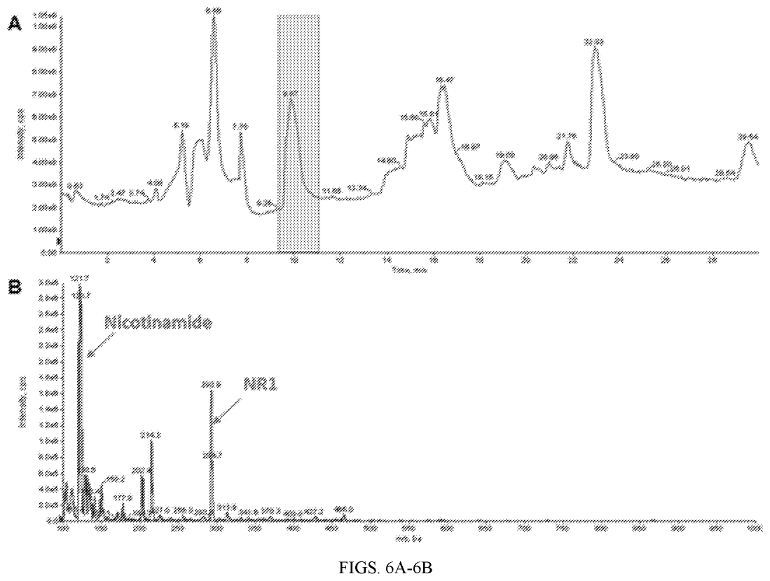

Human Expi293 cells were treated with 1 mM NR1 for 24 hours. The cells were collected and lysed with pre-chilled 10% perchloric acid. The supernatant was collected and neutralized with one-third volum of 3M K2CO3. The supernatant was then subjected analysis by liquid chromatography and mass spectrometry. FIG. 6A shows the reverse-phase liquid chromatography for separation of the cellular extracts. FIG. 6B shows the mass spectrometry of the selected fraction for detection of cellular NR1 analogue.

### Example 16: LC-MS Analysis of NAD⁺1

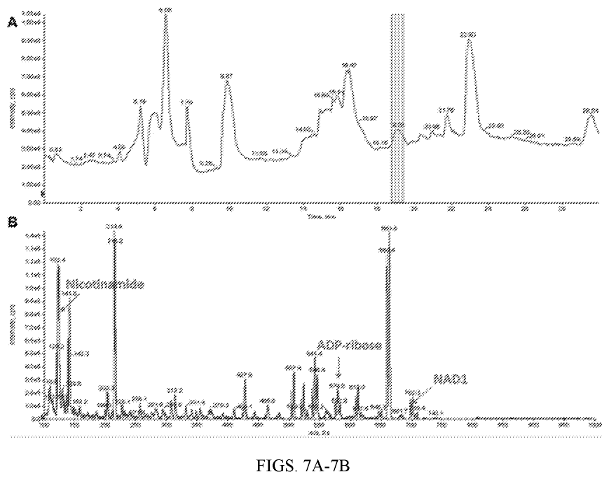

Human Expi293 cells were treated with 1 mm NAD⁺1 for 24 hours. The cells were collected and lysed with pre-chilled 10% perchloric acid. The supernatant was collected and neutralized with one-third volum of 3M K2CO3. The supernatant was then subjected analysis by liquid chromatography and mass spectrometry. FIG. 7A shows the reverse-phase liquid chromatography for separation of the cellular extracts. FIG. 7B shows the mass spectrometry of the selected fraction for detection of cellular NR1 analogue.

### EQUIVALENTS

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. Shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All publications, patent applications, patents, and other references mentioned herein are expressly incorporated by reference in their entirety, including all formulas and figures, to the same extent as if each were incorporated by reference individually. In case of conflict, the present specification, including definitions, will control.

Other embodiments are set forth within the following claims.

1. A compound of Formula (I-A): 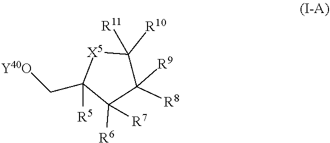
or a tautomer thereof, or an N-oxide of each thereof, or a pharmaceutically acceptable salt of each of the aforementioned, or a pharmaceutically acceptable solvate of each of the foregoing, wherein:
each of R⁵, R⁶, R⁷, R⁸, R⁹, R¹⁰, and R¹¹ independently is a hydrogen, —N₃, a hydroxyl, an optionally substituted C₁-C₁₀ alkyl, an optionally substituted C₂-C₁₀ alkynyl, an optionally substituted C₁-C₁₀ alkoxy, —SR³⁰, an optionally substituted C₆-C₁₀ aryl, an optionally substituted 5-15 membered heteroaryl, or Z;
X⁵ is —S—, —O—, or —NR²⁰-;
each R²⁰ and R³⁰ is independently a hydrogen or an optionally substituted C₁-C₁₀ alkyl;
Z is 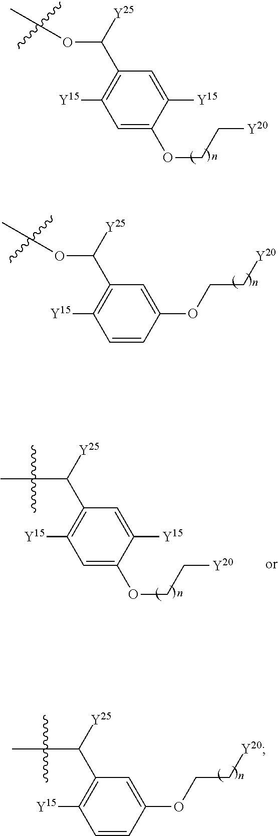
each n is independently 1-4 or 1, 3, or 4;
each Y¹⁵ is independently a hydrogen, —NO₂, a halo, a cyano, a hydroxyl, an optionally substituted C₁-C₆ alkyl, or an optionally substituted C₁-C₆ alkoxy;
each Y²⁵ is independently a hydrogen or an optionally substituted C₁-C₆ alkyl, and
each Y²⁰ is independently selected from the group consisting of: 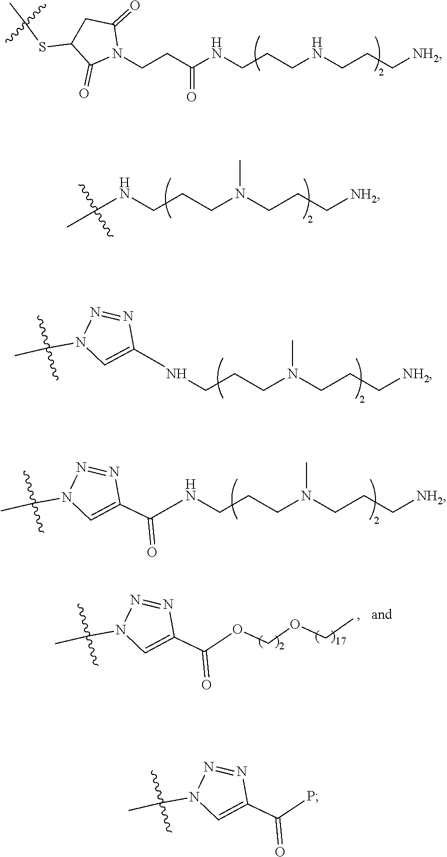
P is a cationic polypeptide of about 5-30 amino acid residues;
Y⁴⁰ is a hydrogen, an optionally substituted C₁-C₆ alkyl, an optionally substituted C₂-C₁₀ alkenyl, an optionally substituted C₂-C₁₀ alkynyl, an optionally substituted C₆-C₁₀ aryl, an optionally substituted 5-15 membered heteroaryl, or -L¹Y³⁵;
L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—;
each R¹⁰⁰ is independently —O^{⊖}, an optionally substituted C₁-C₁₀ alkyl group, or an optionally substituted C₁-C₁₀ alkoxy; and
Y³⁵ is a hydroxyl or an optionally substituted C₁-C₆ alkoxy.

2.-3. (canceled)

4. A compound of Formula (I-B): 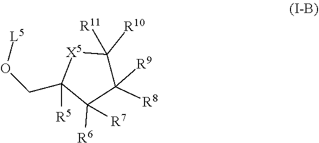
or a tautomer thereof, or an N-oxide of each thereof, or a pharmaceutically acceptable salt of each of the aforementioned, or a pharmaceutically acceptable solvate of each of the foregoing, wherein:
each of R⁵, R⁶, R⁷, R⁸, R⁹, R¹⁰, and R¹¹ independently is a hydrogen, —N₃, a hydroxyl, an optionally substituted C₁-C₁₀ alkyl, an optionally substituted C₂-C₁₀ alkynyl, an optionally substituted C₁-C₁₀ alkoxy, —SR³⁰, an optionally substituted C₆-C₁₀ aryl, an optionally substituted 5-15 membered heteroaryl, or Z;
X⁵ is —S—, —O—, or —NR²⁰-;
each R²⁰ and R³⁰ is independently a hydrogen or an optionally substituted C₁-C₁₀ alkyl;
Z is 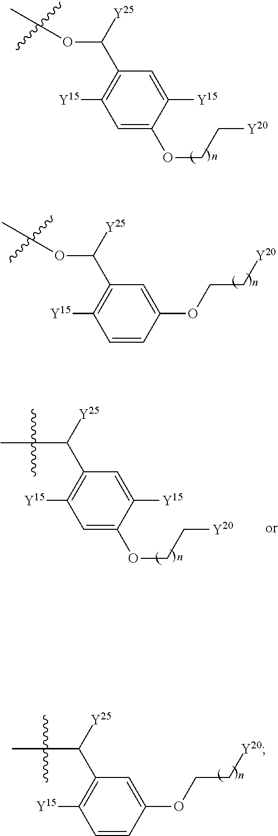
each n is independently 1-4 or 1, 3, or 4;
each Y¹⁵ is independently a hydrogen, —NO₂, a halo, a cyano, a hydroxyl, an optionally substituted C₁-C₆ alkyl, or an optionally substituted C₁-C₆ alkoxy;
each Y²⁵ is independently a hydrogen or an optionally substituted C₁-C₆ alkyl, and
each Y²⁰ is independently selected from the group consisting of: 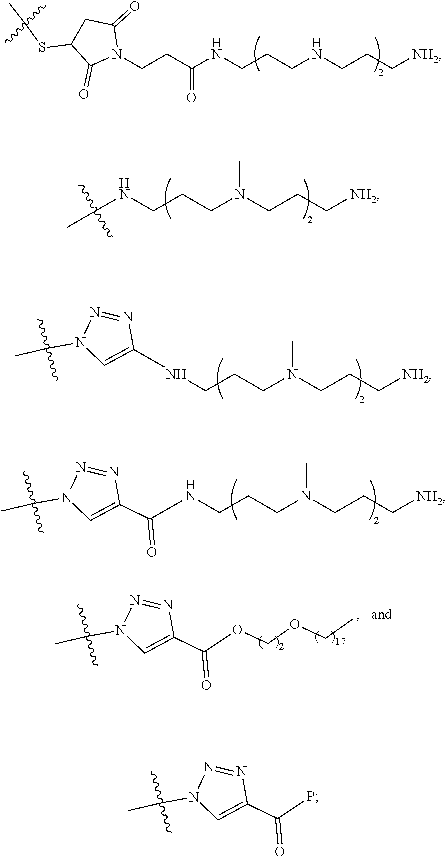
P is a cationic polypeptide of about 5 to 30 amino acid residues in length;
L⁵ is a hydrogen, an optionally substituted C₁-C₆ alkyl, or -L¹Y³⁵;
L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—;
each R¹⁰⁰ is independently —O^{⊖}, an optionally substituted C₁-C₁₀ alkyl group, or an optionally substituted C₁-C₁₀ alkoxy; and
Y³⁵ is a hydroxyl or an optionally substituted C₁-C₆ alkoxy.

5.-6. (canceled)

7. A compound of Formula (I-C): 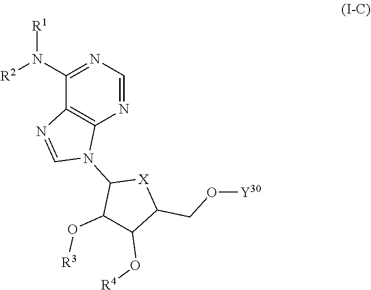
or a tautomer thereof, or an N-oxide of each thereof, or a pharmaceutically acceptable salt of each of the aforementioned, or a pharmaceutically acceptable solvate of each of the foregoing, wherein
each R¹, R², R³, and R⁴ independently is a hydrogen or an optionally substituted C₁-C₆ alkyl or Z;
X is —S—, —O—, or —NR²⁰—;
R²⁰ is a hydrogen or an optionally substituted C₁-C₁₀ alkyl;
Z is 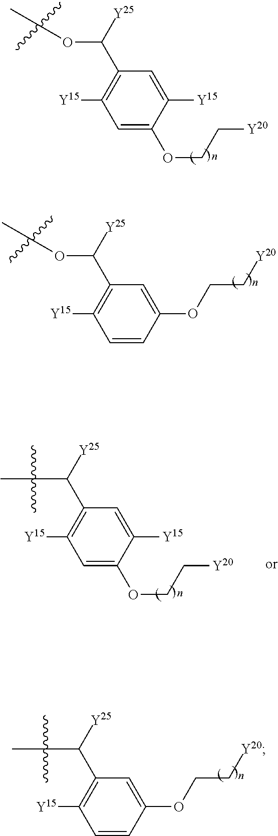
each n is independently 1-4 or 1, 3, or 4;
each Y¹⁵ is independently a hydrogen, —NO₂, a halo, a cyano, a hydroxyl, an optionally substituted C₁-C₆ alkyl, or an optionally substituted C₁-C₆ alkoxy;
each Y²⁵ is independently a hydrogen or an optionally substituted C₁-C₆ alkyl, and
each Y²⁰ is independently selected from the group consisting of: 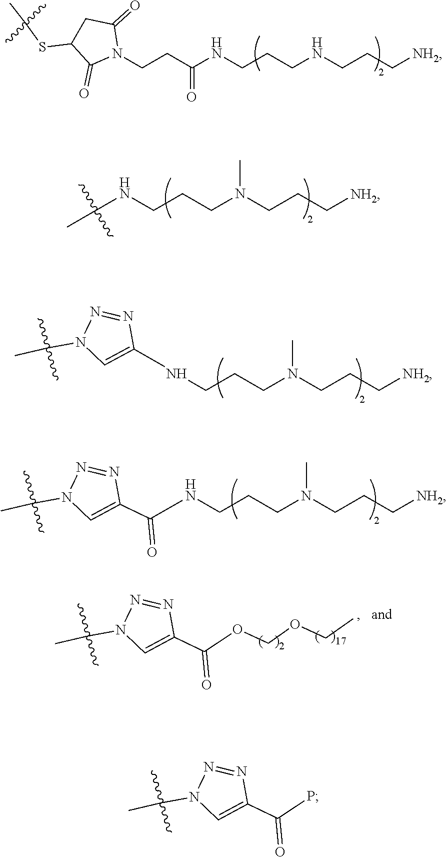
P is a cationic polypeptide of about 5-30 amino acid residues in length;
Y³⁰ is a hydrogen, an optionally substituted C₁-C₆ alkyl, an optionally substituted C₂-C₁₀ alkenyl, an optionally substituted C₂-C₁₀ alkynyl, an optionally substituted C₆-C₁₀ aryl, an optionally substituted 5-15 membered heteroaryl, or -L¹Y³⁵;
L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—;
each R¹⁰⁰ is independently —O^{⊖}, an optionally substituted C₁-C₁₀ alkyl group, or an optionally substituted C₁-C₁₀ alkoxy; and
Y³⁵ is a hydroxyl or an optionally substituted C₁-C₆ alkoxy.

8. (canceled)

9. A compound of Formula (I-D): 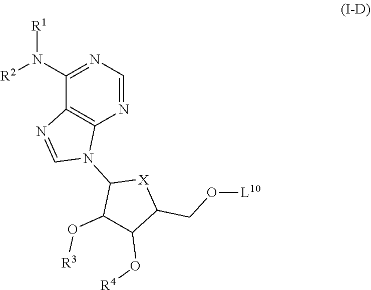
or a tautomer thereof, or an N-oxide of each thereof, or a pharmaceutically acceptable salt of each of the aforementioned, or a pharmaceutically acceptable solvate of each of the foregoing, wherein
each of R¹, R², R³, and R⁴ independently is a hydrogen or an optionally substituted C₁-C₆ alkyl or Z;
X is —S—, —O—, or —NR²⁰—;
R²⁰ is a hydrogen or an optionally substituted C₁-C₁₀ alkyl;
L¹⁰ is a hydrogen, an optionally substituted C₁-C₆ alkyl, or -L¹Y³⁵;
L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—;
each R¹⁰⁰ is independently —O^{⊖}, an optionally substituted C₁-C₁₀ alkyl group, or an optionally substituted C₁-C₁₀ alkoxy;
Y³⁵ is a hydroxyl or an optionally substituted C₁-C₆ alkoxy;
Z is 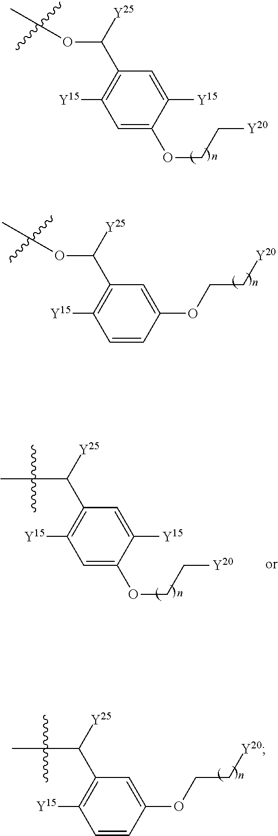
each n is independently 1-4 or 1, 3, or 4;
each Y¹⁵ is independently a hydrogen, —NO₂, a halo, a cyano, a hydroxyl, an optionally substituted C₁-C₆ alkyl, or an optionally substituted C₁-C₆ alkoxy;
each Y²⁵ is independently a hydrogen or an optionally substituted C₁-C₆ alkyl, and
each Y²⁰ is independently selected from the group consisting of: 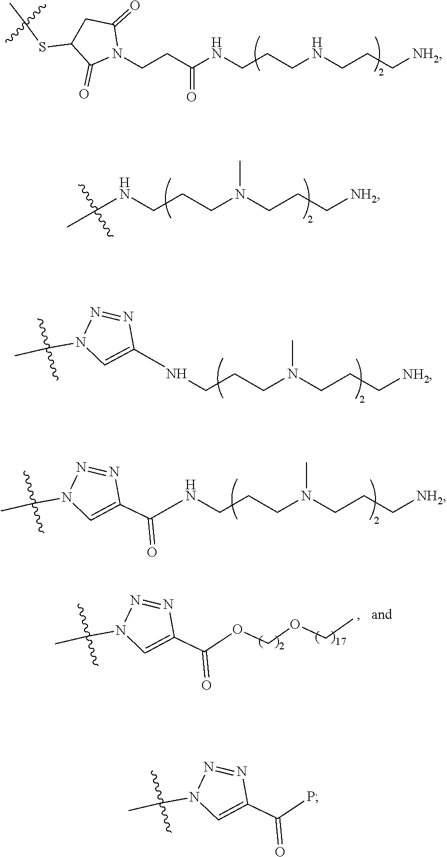
and
P is a cationic polypeptide of about 9-30 amino acid residues in length.

10. (canceled)

11. A compound of Formula (I): 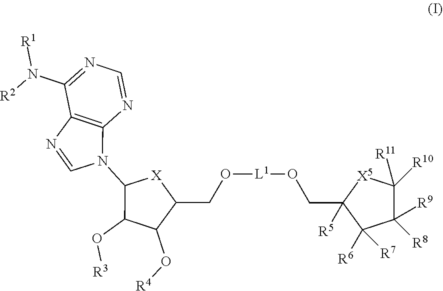
or a tautomer thereof, or an N-oxide of each thereof, or a pharmaceutically acceptable salt of each of the aforementioned, or a pharmaceutically acceptable solvate of each of the foregoing, wherein
each R¹, R², R³, and R⁴ independently is a hydrogen or an optionally substituted C₁-C₆ alkyl or Z;
X is —S—, —O—, or —NR²⁰—;
X⁵ is —S—, —O—, or —NR²⁰-;
L¹ is —PO₂—, —PO₃—PO₂—, —PO₃—PO₃—PO₂—, —P(═O)(R¹⁰⁰)—, —P(═O)(R¹⁰⁰)OP(═O)(R¹⁰⁰)—, or —P(═O)(R¹⁰⁰)OP(═O)(R¹⁰⁰)OP(═O)(R¹⁰⁰)—;
R¹⁰⁰ is —O^{⊖}, an optionally substituted C₁-C₁₀ alkyl group, or an optionally substituted C₁-C₁₀ alkoxy;
each R⁵, R⁶, R⁷, R⁸, R⁹, R¹⁰, and R¹¹ independently is a hydrogen, —N₃, a hydroxyl, an optionally substituted C₁-C₁₀ alkyl, an optionally substituted C₂-C₁₀ alkynyl, an optionally substituted C₁-C₁₀ alkoxy, —SR³⁰, an optionally substituted C₆-C₁₀ aryl, an optionally substituted 5-15 membered heteroaryl, or Z;
Z is 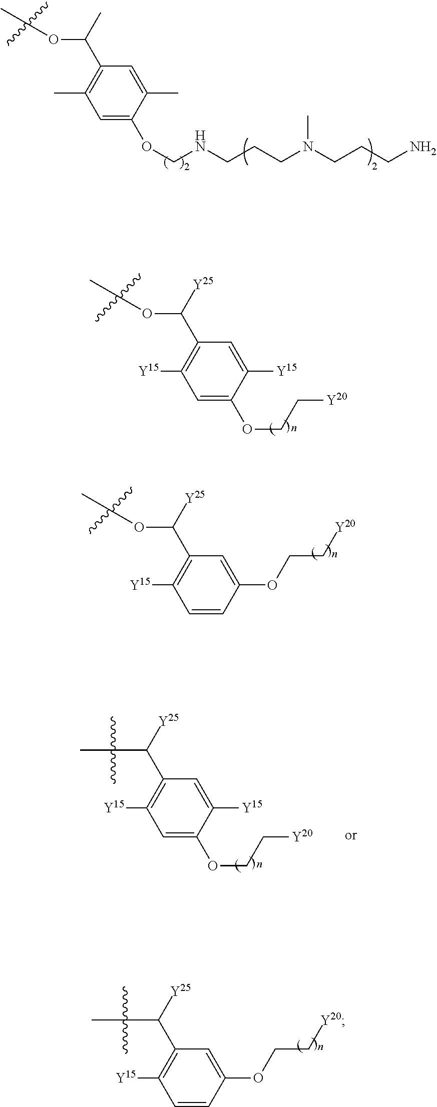
each n is independently 1-4 or 1, 3, or 4;
each Y¹⁵ is independently a hydrogen, —NO₂, a halo, a cyano, a hydroxyl, an optionally substituted C₁-C₆ alkyl, or an optionally substituted C₁-C₆ alkoxy;
each Y²⁵ is independently a hydrogen or an optionally substituted C₁-C₆ alkyl, and
each Y²⁰ is independently selected from the group consisting of: 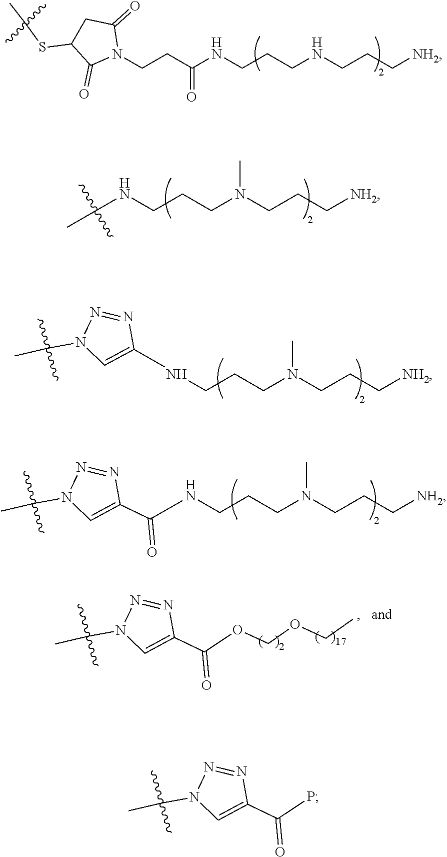
P is a cationic polypeptide of about 9-30 amino acid residues in length; and
each R²⁰ and R³⁰ is independently a hydrogen or an optionally substituted C₁-C₁₀ alkyl.

12.-30. (canceled)

31. A compound selected from Table 1, Table 2, Table 3 or Table 4.

32.-34. (canceled)

35. A method of monitoring and/or tracking ADP-ribosylation in a cell or sample comprising a PARP enzyme, the method comprising: contacting the cell or sample with a compound of claim 1; labeling a PARP catalyzed reaction product; and detecting the product of the PARP catalyzed reaction, thereby monitoring and/or tracking ADP-ribosylation.

36.-37. (canceled)

38. A method of purifying a PARP substrate protein, the method comprising: contacting a cell or sample comprising a PARP with a compound of claim 1; labeling a PARP catalyzed reaction product with an affinity label, and purifying the product of the PARP catalyzed reaction.

39. A method of identifying a protein as a substrate for PARP, the method comprising contacting a cell or sample comprising the PARP with a compound of claim 1; labeling a PARP catalyzed reaction product with an affinity label; and purifying and characterizing the product of the PARP catalyzed reaction.

40.-41. (canceled)

42. A method of labeling a PARP substrate protein, the method comprising contacting a cell or sample comprising PARP with a compound of any one of claim 1; and labeling a product of a PARP catalyzed reaction.

43. (canceled)

44. A kit comprising a compound of claim 1, and instructions for use.
